(19) United States
(12) Patent Application Publication
(10) Pub. No.: US 2025/0275995 A1
(43) Pub. Date: Sep. 4, 2025

(54) NICOTINAMIDE RIBOSIDE DOSAGE REGIMEN FOR TREATING PARKINSON'S DISEASE

(71) Applicant: VESTLANDETS INNOVASJONSSELSKAP AS (VIS), Bergen (NO)

(72) Inventors: Charalampos TZOULIS, Bergen (NO); Christian DÖLLE, Bergen (NO)

(21) Appl. No.: 18/858,964

(22) PCT Filed: Apr. 26, 2023
(86) PCT No.: PCT/EP2023/060962

§ 371 Date: Oct. 22, 2024

(30) Foreign Application Priority Data
Apr. 26, 2022 (GR) 20220100339

(51) Int. Cl. A61K 31/706; A61K 9/48; A61K 31/137; A61K 31/195; A61P 25/16

(52) U.S. Cl. CPC A61K 31/706; A61K 9/485; A61K 9/4858; A61K 9/4866; A61K 31/137; A61K 31/195; A61P 25/16

(57) ABSTRACT

The present invention generally relates to the treatment of Parkinson's disease (PD) in humans. More particularly, it relates to nicotinamide riboside (NR) for use in a method for treatment of Parkinson's disease in a human subject characterized by a particular dosage regimen; pharmaceutical compositions; and dosage forms, which can be for use in or as treatment of Parkinson's disease.

### FIELD OF THE INVENTION

The present invention generally relates to the treatment of Parkinson's disease (PD) in humans. More particularly, it relates to nicotinamide riboside (NR) for use in a method for treatment of Parkinson's disease in a human subject characterized by a particular dosage regimen; pharmaceutical compositions; and dosage forms, which can be for use in or as treatment of Parkinson's disease.

### BACKGROUND ART

PD affects 1-2% of the population above the age of 65, and is a major cause of death and disability with a devastating global socioeconomic impact. In Europe alone, PD affects an estimated 1.2 million people and has a cost of €14 billion per year. Current treatments for PD are purely symptomatic and have no impact on disease progression. As a result, patients confront a future of progressive disability, early institutionalization, and premature death. Since demographic studies show that patient numbers will continue to grow, effectively doubling by 2040, our failure to make any significant impact to halt or delay disease progression means that PD is now a major challenge to health care and society.

Increasing evidence supports that boosting cellular levels of nicotinamide adenine dinucleotide (NAD) confers neuroprotective effects in both healthy aging and neurodegeneration. NAD, which constantly shuttles between its oxidized (NAD⁺) and reduced (NADH) state, is an essential cofactor for metabolic redox reactions, including mitochondrial respiration. Furthermore, NAD⁺ is substrate to vital signaling reactions involved in DNA repair, histone- and other protein deacylation, and second messenger generation. These reactions consume NAD⁺ at high rates, requiring constant replenishment via NAD biosynthesis. NAD levels have been shown to decline with age and this is believed to contribute to age-related diseases. Increasing the NAD replenishment rate (e.g., via supplementation of precursors), and/or enhancing the NAD⁺/NADH ratio (e.g., via caloric restriction) have shown beneficial effects on life- and health span in multiple model systems, and evidence of neuroprotection in models of neurodegeneration and other age-related diseases. Enhancing NAD replenishment could potentially help ameliorate several major processes implicated in the pathogenesis of PD, including mitochondrial respiratory dysfunction, neuroinflammation, epigenomic dysregulation, and increased neuronal DNA damage.

NAD can be replenished via supplementation of nicotinamide riboside (NR), a vitamin B3 molecule and biosynthetic precursor of NAD. NR has undergone extensive preclinical testing, and is well tolerated by adult humans. NR administered to human patients with PD at 1000 mg per day over 30 days has excellent compliance, tolerability and no signs of toxicity or adverse side effects in PD, achieves brain penetration, and is associated with clinical improvement of PD.

However, little is known about the safety, tolerability and efficacy of NR at high dosages. The biological and clinical effects of high doses of NR (e.g., more than 1000 mg daily) have not been explored in human patients suffering from PD. Therefore, it has been unknown so far whether improved biological and clinical responses can be expected or achieved by escalating the dose. Additionally, depending on individual factors, NAD response may reflect variability in cerebral NAD metabolism (i.e., variation in the rate of NAD-synthesis or consumption). Establishing an appropriate dosage regimen is therefore important, so that NR-therapy can be correctly dosed and tailored to individual patients to achieve an optimal neurometabolic response, while maintaining acceptable safety and tolerability.

Thus, a need exists for an improved treatment of PD in human patients addressing one or more of the above needs.

### SUMMARY OF THE INVENTION

As a solution, the present invention provides Nicotinamide riboside (NR) for use in a method for treatment of Parkinson's Disease (PD) in a human subject, comprising the following steps, in this order:
measuring one or more biological parameter(s) of the subject at timepoint V1;
administering NR to the subject at a first dose over a first period until timepoint V2;
measuring one or more biological parameter(s) at timepoint V2, wherein:
   if a biological response is achieved, the first dose is maintained, and
   if a biological response is not achieved, the dose is increased to a second dose;
administering NR to the subject at the second dose over a second period until timepoint V3;
measuring one or more biological parameter(s) at timepoint V3, wherein:
   if a biological response is achieved, the dose is maintained, and
   if a biological response is not achieved, the dose is increased to a third dose; and
administering NR to the subject at the third dose over a third period until timepoint V4;
measuring one or more biological parameter(s) at timepoint V4, wherein:
   if a biological response is achieved at V4, the dose is maintained, and
   if a biological response is not achieved at V4, the treatment is discontinued;
wherein:
the biological parameter(s) are selected from: cerebral NAD levels, cerebrospinal fluid (CSF) NAD and/or related metabolite levels, blood NAD levels, and NR-related metabolic pattern (NRRP) expression; and
the biological response is defined as change in one or more of the measured biological parameter(s) relative to the respective previous timepoint and/or relative to timepoint V1.

The present invention further provides NR for use in a method for treating Parkinson's Disease (PD) in a human subject at a dose of more than 3000 mg daily.

The present invention further provides a pharmaceutical composition for use in the method as 250 defined above, the composition comprising NR and optionally one or more pharmaceutically acceptable excipient(s).

The present invention further provides a dosage form for use in a method as defined above, the dosage form comprising NR or a pharmaceutical composition as defined above.

The present invention further provides a dosage form comprising >2000 mg NR per unit, and optionally one or more pharmaceutically acceptable excipient(s).

### BRIEF DESCRIPTION OF THE DRAWINGS

FIG. 1 shows results of a randomized, double blinded trial, aiming to assess the tolerability, cerebral bioavailability and molecular effects of NR therapy (1000 mg per day for 30 days) in Parkinson's Disease (PD). A-C: 31P-MRS was performed at baseline (v1) and after 30 days of treatment (v2). A: exemplary data from one subject showing voxel position. Spectra were acquired for each grid position. B (top): average processed spectra from multiple voxels (black) and the model fit (red). B (bottom): the model fit is composed of the convolution of all spectral contributions of a simulated dataset fitted to the experimental data. Arrow shows the NAD+/NADH spectral peaks. PCr: phosphocreatine. C: comparison of cerebral NAD levels in the placebo (PL) and NR groups at baseline (v1) and visit-2 (v2). The Y-axis shows measured levels of NAD normalized to α-ATP (which remain unchanged and stable by the intervention). Individual subjects are indicated by dots. Connecting lines show the change between v1 and v2. The black points and lines show the mean of each group. The treatment group shows a highly significant increase in cerebral NAD levels (*paired t-test p=0.016), whereas no difference is observed in the placebo group (paired t-test p=0.75). D: FDG-PET data from all subjects in the NR-group, showing the mean NR-Related Pattern (NRRP, top panel), PD-related pattern (PRDP, middle panel), and the overlap of the two (bottom panel). NR partially ameliorates the striatal and thalamic hypermetabolism of PD. E: Between visit changes showing increased NRRP expression in the NR versus the placebo group. The red line indicates mean values before and after the treatment, black lines indicate individuals with a positive NAD response in the MRS analysis, grey lines indicate individuals without an NAD response in the MRS (*p=0.027, permutation test, 1000 iterations). F: the NR-induced change in metabolic pattern (delta NRRP) shows a strong negative correlation (P<0.01) with the decrease in the UPDRS score (delta UPDRS). G: Metabolomics in PBMCs, muscle, and CSF. NR-induced metabolic changes include highly significant (p<<0.001 for all tests) increases in the acid form of NAD (NAAD), and nicotinamide (Nam) degradation products: methyl-Nam (Me-Nam) and the methyl pyridone (Me-2-PY).

FIG. 2 shows a general outline of a dosage regimen according to the present invention. [symbol] denotes measurement point; denotes escalation dose, i.e., a dosage which is higher than the one administered during the period prior to the last measurement point; . . . denotes maintenance dose, i.e., a dosage which is established by the method as described herein, and maintained for the future treatment of the patient.

FIG. 3 shows an exemplary outline of a specific dosage regimen according to the present invention (dose individualization flowchart for a particular patient).

FIG. 4 shows the scheme for drug administration and biological response measurement, as used in Example 1 (A); and Example 2 (B).

FIG. 5 shows flowchart of titration of dopaminergic treatment prior to inclusion to studies of Examples 1 and 2.

FIG. 6: Example 3: CONSORT flow diagram. 26 patients with PD were screened for inclusion and 20 enrolled in the study. All randomized participants completed the study.

FIG. 7: High dose NR supplementation improves MDS-UPDRS scores in individuals with PD. The plots show changes in MDS-UPDRS scores for the placebo (blue) and NR (red) group, both for total MDS-UPDRS (A) and parts I-IV of the MDS-UPDRS scale (B-E). A subset of patients (NR group: n=8, placebo group: n=8) with similar intervals since levodopa administration at the last visit was analyzed as well (F). *p<0.05, **p<0.005, ***p<0.0005.

FIG. 8: High dose NR increases NAD and NADP metabolites in whole blood samples. The plots show changes from baseline (V1) to last visit (V7) in the placebo (blue) and NR (red) group for the indicated metabolites in flash frozen whole blood samples. *p<0.05, **p<0.005, ***p<0.0005.

FIG. 9: High dose NR augments the NAD metabolome. Plots show data from flash frozen whole blood (A-Q) and urine (S-Y) samples analyzed by LC-MS for the indicated metabolites in the placebo (blue) and NR (red for whole blood samples, yellow for urine samples) group. NAD⁺: Nicotinamide adenine dinucleotide (oxidized); NADP⁺: Nicotinamide adenine dinucleotide phosphate (oxidized); Me-Nam: 1-methyl nicotinamide; NAAD: nicotinic acid-adenine dinucleotide; Me-2-PY: N1-methyl-2-pyridone-5-carboxamide; Nam: Nicotinamide; Nam N-oxide: Nicotinamide N-oxide; ADPR: ADP-ribose; NAR: Nicotinic acid riboside; NR: Nicotinamide riboside; NMN: Nicotinamide mononucleotide; NA: Nicotinic acid, ATP: Adenosine triphosphate; ADP: Adenosine diphosphate; AMP: Adenosine monophosphate, GDP: Guanosine diphosphate; GTP: Guanosine triphosphate. *p<0.05, **p<0.005, ***p<0.0005.

FIG. 10: High dose NR supplementation mildly increases homocysteine in serum, but not whole blood, and does not lead to methylgroup pool depletion. A-C. The plots show changes of serum HCy on the individual (A) and group (B) level from baseline (V1) to last visit (V7), and mean serum HCy levels at all indicated visits (C). Reference serum values for HCy: >60 years of age: <15.9 μM; 41-60 years of age: <14.2 μM. D-G: The plots show the levels of HCy (D), SAM (E), SAH (F), and the SAM/SAH ratio (G) in whole blood samples at baseline (V1) and last visit (V7). H-I: Plots show Urine levels of HCy and SAM. J-K: Plots show changes of betaine (J) and methionine (K) in whole blood samples. Placebo group: blue; NR group: red. HCy: homocysteine; SAM: S-adenosyl methionine; SAH, S-adenosyl homocysteine. *p<0.05, **p<0.005, ***p<0.0005.

FIG. 11: Overview of NAD metabolism in interaction with methyl-group metabolism. The figure shows various routes of NAD biosynthesis, and examples of NAD degradation leading to Nam production. If not recycled, Nam is methylated to Me-Nam, in a reaction catalysed by NNMT and requiring SAM as methyl-group donor. The other product of this reaction, SAH, is further converted to HCy, and subsequently regenerated to methionine in the methionine cycle. Me-Nam: 1-methyl nicotinamide; NAAD: Nicotinic acid adenine dinucleotide; Nam: Nicotinamide; Nam N-oxide: Nicotinamide N-oxide, cADPR: Cyclic ADP-ribose; ADPR: Adenosine diphosphate ribose; NAR: Nicotinic acid ribonucleoside; NR: Nicotinamide riboside, NMN: Nicotinamide mononucleotide; NA: Nicotinic acid, Me-2-PY: N1-methyl-2-pyridone-5-carboxamide; Me-4-PY: N-methyl-4-pyridone-5-carboxamide; NAMN: Nicotinic acid mononucleotide; SAM: S-adenosyl methionine; SAH: S-adenosyl homocysteine, NNMT: Nicotinamide N-methyltransferase, MS: Methionine synthase, CD38: Cluster of differentiation 38/cyclic ADP-ribose hydrolase; Methyl-THF: Methyl-tetrahydrofolate; THF: Tetrahydrofolate; BHMT: Betaine homocysteine methyltransferase; Di-Gly: Dimethyl glycine; Bet: Betaine.

FIG. 12: Example 4: Blood NAD⁺ response following consumption of 1200 mg NR daily (600 mg×2) for eight days. Day 0 is the baseline, NAD Precursor uptake was initiated in the evening of day 0, after baseline measurements. The last dose was received on the morning of day 8. Y-axis: whole blood NAD⁺ levels in micromolar (μM). X-axis: time in days. Each individual is represented by a different symbol and color.

FIG. 13: Blood NAD⁺ response following consumption of 1200 mg NMN daily (600 mg×2) for eight days. Day 0 is the baseline, NAD Precursor uptake was initiated in the evening of day 0, after baseline measurements. The last dose was received on the morning of day 8. Y-axis: whole blood NAD⁺ levels in micromolar (μM). X-axis: time in days. Each individual is represented by a different symbol and color.

FIG. 14. Brain NAD response, measured by 31P-MRS, following consumption of 1200 mg (600 mg×2) NR (individuals 1-3) or NMN (individuals 4-6) daily for eight days. BL: baseline (Day 0). Day-9 is the time of maximal NAD increase in the blood of NR recipients. Day-8 is the time of maximal NAD increase in the blood of NMN recipients. The last dose was received on the morning of day 8. Y-axis: brain NAD/alpha-ATP ratio.

FIG. 15: Individual brain NAD response, measured by 31P-MRS, following consumption of 1200 mg (600 mg×2) NR daily for eight days. Day 0 is the baseline. The last dose was received on the morning of day 8. Y-axis: brain NAD/alpha-ATP ratio. Each individual is represented by a different symbol and color.

FIG. 16: Individual brain NAD response, measured by 31P-MRS, following consumption of 1200 mg (600 mg×2) NMN daily for eight days. Day 0 is the baseline. The last dose was received on the morning of day 8. Y-axis: brain NAD/alpha-ATP ratio. Each individual is represented by a different symbol and color.

FIG. 17: Brain NAD response, measured by 31P-MRS, following consumption of 1200 mg (600 mg×2) NR for eight days. Day 0 is the baseline. The last dose was received on the morning of day 8. Y-axis: brain NAD/alpha-ATP ratio. The mean of all individuals per time point is shown. Error bars show standard deviation.

FIG. 18: Brain NAD response, measured by 31P-MRS, following consumption of 1200 mg (600 mg×2) NMN for eight days. Day 0 is the baseline. The last dose was received on the morning of day 8. Y-axis: brain NAD/alpha-ATP ratio. The mean of all individuals per time point is shown. Error bars show standard deviation.

### DETAILED DESCRIPTION OF THE INVENTION

Nicotinamide Riboside (NR) is approved for human use, has undergone extensive preclinical testing, and is Generally Recognized as Safe (GRAS) for use in food products by the United States Food and Drug Administration and by the European Food Safety Authority. NR is well tolerated with no evidence of toxicity in adult humans with doses up to at least 2000 mg daily. However, no clinical results or other hints have been published so far concerning the safety, tolerability and efficacy of NR in human patients with PD at doses higher than 1000 mg daily.

The present inventors completed a phase I randomized, double blinded trial, aiming to assess the tolerability, cerebral bioavailability and molecular effects of NR therapy in PD. A total of 30 individuals with newly diagnosed, drug-naïve PD were randomized to NR 500 mg×2/day or placebo for 30 days (Cell Metab. 2022 Mar. 1; 34(3):396-407.e6). The study showed promising results, which are summarized and further analyzed in the context of the present invention as outlined below.

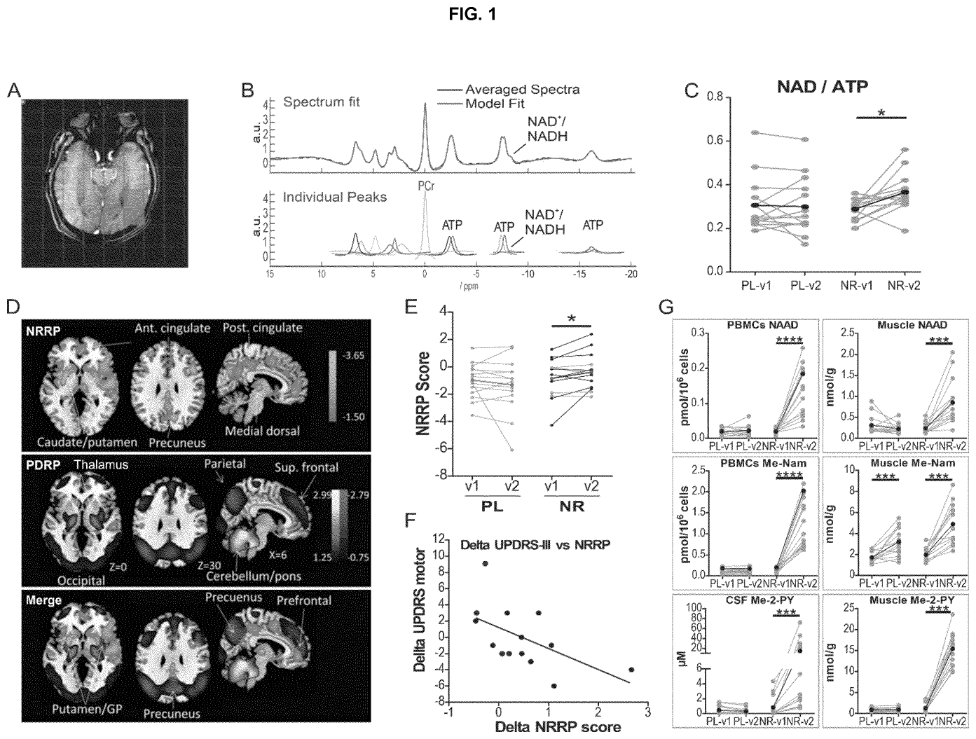

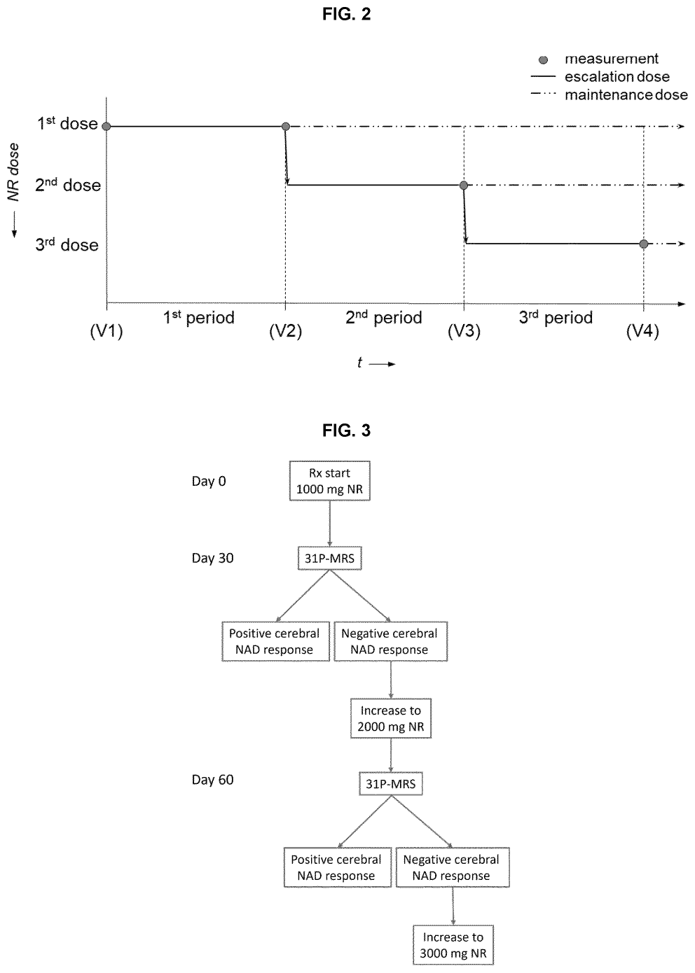

NR is well-tolerated. NR administered 1000 mg per day has excellent compliance, tolerability and no signs of toxicity or adverse side effects in PD. NR achieves brain penetration. In vivo measurement of cerebral NAD levels using phosphorus magnetic resonance spectroscopy (31P-MRS) of the brain showed a highly significant (paired t-test: P=0.016) increase in cerebral NAD levels in the NR group, while no change was observed in the placebo group (FIG. 1A-C). Cerebral penetration was further validated by detecting the metabolite Me-2-PY in the CSF of participants receiving NR, but not placebo (FIG. 1G). While a significant NR-induced increase in cerebral NAD levels was detected at the group level, this effect was not uniform at the individual level. The magnitude of the cerebral NAD-increase showed high interindividual variation (FIG. 1C).

However, three patients showed no evidence of cerebral NAD response, despite a clear peripheral metabolic response, confirming treatment compliance and an impact on the NAD metabolome, in CSF, blood, and muscle. Thus, the variable cerebral NAD response observed by ³¹P-MRS may reflect interindividual variability in cerebral penetration and/or cerebral NAD metabolism. We will henceforth refer to individuals susceptible to NR-induced increase in cerebral NAD levels, detectable by ³¹P-MRS, as “MRS-responders”, and to individuals showing no NR-induced increase in cerebral NAD levels, detectable by ³¹P-MRS, as “MRS-non-responders”.

NR is associated with clinical improvement of PD. NR at 1000 mg daily was associated with a significant decrease in the total MDS-UPDRS (I-III) score between visits (mean decrease: 2.33±2.35; paired t-test: p=0.017). No significant change was found in the MDS-UPDRS (total or subsections I-III) in the NR or placebo groups. However, a trend for decreased MDS-UPDRS was seen in the subgroup of ten NR-recipients who showed increased cerebral NAD levels (mean decrease 1.9±2.78, paired t-test: p=0.071), and this reached statistical significance when only the nine individuals showing >10% increase in cerebral NAD levels were considered (mean decrease 2.33±2.35; paired t-test: p=0.017).

NR has a major impact on cerebral metabolism. ¹⁸F-fluorodeoxyglucose positron emission tomography (FDG-PET), performed at baseline and 30 days of treatment, revealed that NR altered cerebral metabolic activity. The analysis revealed a significant ordinal trend pattern (i.e., metabolic network), which was represented by the first principal component (PC1), accounting for 20.6% of the variance in the paired data. This novel NR-related metabolic pattern (NRRP) was characterized by multiple regional metabolic changes, including bilateral metabolic reductions in the caudate and putamen, extending into the adjacent globus pallidus, and in the thalamus (FIG. 1D-E). Interestingly, the NRRP overlapped spatially with the Parkinson's Disease-Related Pattern (PDRP), and changes in NRRP expression in the NR group resulted in partial normalization of the striatal and thalamic hypermetabolism (FIG. 1D), typically characterizing the PD brain. Furthermore, changes in NRRP expression in the NR group correlated significantly (r=−0.59, p=0.026) with a decrease of the UPDRS ratings recorded at the time of PET (FIG. 1F). These results indicate that NR ameliorates the cerebral metabolic pattern of PD, and this is associated with significant clinical improvement.

NR has widespread metabolic and regulatory effects. Metabolomics revealed highly significant increase in NAD-related metabolites in blood, muscle and CSF (FIG. 1G), indicating that NR supplementation boosts NAD metabolism across tissues. Intriguingly, RNA-sequencing in blood and muscle biopsy showed a highly significant (FDR<10⁻⁸) upregulation of the mitochondrial, proteasomal and lysosomal pathways in the NR group. These findings indicate that NR supplementation increases both mitochondrial respiration and proteostasis—two hallmark pathogenic processes involved in PD.

In view of the above, an objective underlying the present invention aims at determining an optimal biological dose of NR in PD and to further explore its neuroprotective potential, in order to maximize its clinical benefit and impact. Specifically, the following knowledge gaps are addressed by the present invention.

Optimal Biological Dose (OBD) of NR in PD. We define the OBD of NR as the dose required to achieve: maximal cerebral NAD increase (measured by 31P-MRS or CSF metabolomics), or maximal expression increase in the NRRP (measured by FDG-PET), or maximal proportion of MRS-responders, preferably in the absence of unacceptable toxicity. While the above-mentioned Phase I study showed significant biological and clinical effects with 1000 mg NR daily, higher doses have not been explored in PD. Therefore, it unknown in the prior art whether improved biological and clinical responses can be achieved by escalating the dose. Moreover, the Phase I study showed that the NR-mediated increase in cerebral NAD-levels, and accompanying metabolic and clinical response, are not universal and vary across individuals. The fact that all NR-recipients showed a robust metabolic response in blood, muscle and CSF, suggests that the variable cerebral NAD response may reflect interindividual variability in cerebral NAD metabolism (i.e., variation in the rate of NAD-synthesis or consumption). It is likely that such differences can be modulated by varying the substrate concentration (i.e., the intake dose of NR). This question is critical to address, so that NR-therapy can be correctly dosed and tailored to individual patients to achieve an optimal neurometabolic response. Without wishing to be bound by any theory, our results suggest that a dose of ×2 daily is adequate, and that a less frequent dosing (e.g., once daily) may be feasible too.

Dose response of the clinical effect. The NADPARK study showed that NR was associated with a clinical improvement, in the form of UPDRS decrease, and this correlated significantly with the increase in cerebral NAD levels and brain metabolic network (NRRP) change. These findings suggest that NR ameliorates neuronal function in PD resulting in symptom improvement. Exploring the dose-responsiveness of this effect by the methods described herein allow to confirm the clinical impact of NR, allow to account for symptomatic effects in neuroprotection trials, and determine the optimal clinical dose of NR (on a global and/or individual level).

Dose-dependency of metabolic response to NR therapy. The NADPARK study showed that 1000 mg NR daily augmented the NAD metabolome in PBMC, muscle and CSF. While higher doses have not been explored in PD, there is a reasonable expectation that improved metabolic responses can be achieved by escalating the dose.

In view of the above, and without wishing to be bound by theory, it is contemplated that for at least a significant fraction of the patient population, improved biological, clinical and metabolic responses can be achieved by escalating the dose, rather than plateauing at dosages above 1000 mg daily. Our results show that it takes more than 8 days to reach a steady heightened state of NAD-metabolism in blood and bran with NR supplementation. Our results also show that there are great interindividual responses, which may be due to the person's genetics and/or the microbiome composition.

Enhancement of proteostasis in PD by NR therapy. Impaired proteostasis plays a central role in PD and other neurodegenerative disorders, including Alzheimer's disease (AD) and amyotrophic lateral sclerosis (ALS). Our transcriptomic analyses in the NADPARK study indicated that NR therapy may enhance proteostasis by inducing the expression of both proteasomal and lysosomal pathways. If confirmed, this would suggest that NR targets multiple major processes implicated in the pathophysiology of PD, including mitochondrial respiratory dysfunction, oxidative damage, lysosomal and proteasomal impairment, and neuroinflammation. Without wishing to be bound by theory, it is thus contemplated that higher NR doses can induce stronger induction of proteostasis in PD.

Influence of NR therapy on histone acetylation status. We have recently shown that genome-wide histone hyperacetylation and altered transcriptional regulation occur in the brain of individuals with PD (Taker L, Tran G T, Sundaresan J, et al. Genome-wide histone acetylation analysis reveals altered transcriptional regulation in the Parkinson's disease brain. Molecular Neurodegeneration 2021; 16(1):31). Increasing neuronal NAD levels would boost the activity of the NAD-dependent histone deacetylases of the sirtuin family, potentially, ameliorating histone hyperacetylation in PD. Without wishing to be bound by theory, it is thus contemplated that NR-therapy influences histone acetylation status in PD, and whether this effect is dose-dependent.

Decrease of neuroinflammation. It is known that NR has anti-inflammatory properties in peripheral tissues (Elhassan Y S, Kluckova K, Fletcher R S, et al. Nicotinamide Riboside Augments the Aged Human Skeletal Muscle NAD⁺ Metabolome and Induces Transcriptomic and Anti-inflammatory Signatures. Cell Rep 2019; 28(7):1717-1728.e6). The results of the Phase I study described above suggest that NR also downregulates multiple inflammatory cytokines in the central nervous system. Without wishing to be bound by theory, it is thus contemplated that higher NR doses can induce stronger anti-inflammatory effects in PD.

Alteration of methylation metabolism by NR therapy. In theory, NAD replenishment via NR administration could decrease/deplete the cellular methylation capacity. NR boosts the NAD-metabolome, leading to increased production of the degradation product nicotinamide (NAM), which is eliminated via methylation to MeNAM, Me-2-PY, and Me-4-PY, excreted in the urine. Synthesis of Me-Nam requires the methyl-donor S-adenosylmethionine (SAM). This, in turn, could limit SAM availability for other essential methylation reactions, such as DNA and histone methylation, and neurotransmitter synthesis, including dopamine. Thus, in theory, NR would cause an increased consumption of SAM, limiting methylation reactions and generating higher levels of homocysteine. Such a phenomenon would be a particular concern for the ˜60% of the population that carries MTHFR variants that reduce the efficiency of methylation. The Phase I study mentioned above showed no change in serum homocysteine levels, or any other evidence of methylation depletion associated with NR 1000 mg daily. However, with higher NR doses, such effects may occur in certain patient sub-population(s).

Influence on the gut microbiome in PD by NR therapy. Current evidence suggests that the gut microbiome is involved in the pathogenesis of PD (Romano S, Savva G M, Bedarf J R, et al. Meta-analysis of the Parkinson's disease gut microbiome suggests alterations linked to intestinal inflammation. npj Parkinsons Dis. 2021; 7(1):1-13). Without wishing to be bound by theory, it is thus contemplated that NR therapy may beneficially affect the gut microbiome in PD restoring normal patterns.

The single-center randomized double-blinded placebo-controlled trials as described in the Examples address the above questions. Our findings suggest that NR can be useful as a safe and tolerable neuroprotective therapy in PD in human patients. Specifically, tolerability and efficacy of NR at higher dosages, associated with improved biological and clinical responses, may be achieved by escalating the dose by using the dosage regimens and method steps disclosed herein. Additionally, the present invention allows to account for individual variability in cerebral NAD metabolism and find an individually appropriate dosage for a certain patient, to achieve an optimal neurometabolic response, while maintaining acceptable safety and tolerability.

The present invention provides the following aspects and embodiments.

### NR for Use in Methods for Treatment of Parkinson's Disease (PD)

In a first aspect, the present invention provides nicotinamide riboside (NR) for use in a method for treatment of Parkinson's Disease (PD) in a human subject, comprising the following steps, in this order:
measuring one or more biological parameter(s) of the subject at timepoint V1;
administering NR to the subject at a first dose over a first period until timepoint V2;
measuring one or more biological parameter(s) at timepoint V2, wherein:
   if a biological response is achieved, the first dose is maintained, and
   if a biological response is not achieved, the dose is increased to a second dose;
administering NR to the subject at the second dose over a second period until timepoint V3;
measuring one or more biological parameter(s) at timepoint V3, wherein:
   if a biological response is achieved, the dose is maintained, and
   if a biological response is not achieved, the dose is increased to a third dose; and
administering NR to the subject at the third dose over a third period until timepoint V4;
measuring one or more biological parameter(s) at timepoint V4, wherein:
   if a biological response is achieved at V4, the dose is maintained, and
   if a biological response is not achieved at V4, the treatment is discontinued;
wherein:
the biological parameter(s) are selected from: cerebral NAD levels, cerebrospinal fluid (CSF) NAD and/or related metabolite levels, blood NAD levels, and NR-related metabolic pattern (NRRP) expression; and the biological response is defined as change in one or more of the measured biological parameter(s) relative to the respective previous timepoint and/or relative to timepoint V1.

As used herein, the Nicotinamide riboside (NR), the pharmaceutical composition and the dosage for use in a method for treatment of PD may also refer to a method for treatment of PD in a human subject in need thereof, the method comprising the steps as described herein; or to use of NR in the manufacture of a medicament for treatment use treatment of PD, the treatment comprising as described herein.

According to an embodiment, the first dose is in the range of 750 to 3000 mg daily, preferably 1000 to 2000 mg daily, more preferably 1000 to 1500 mg daily, most preferably 1000 mg daily.

According to an embodiment, the first period is up to 16 weeks, preferably 2 to 12 weeks, more preferably 3-10 weeks, even more preferably 4-8 weeks, most preferably 4 weeks. Alternatively, the first period is up to 4 months, preferably 0.5 to 3 months, more preferably 1.5-2.5 months, even more preferably 1-2 months, most preferably 1 month.

According to an embodiment, the second dose is in the range of 1000 to 4000 mg daily, preferably 1000 to 3000 mg daily, more preferably 1500 to 2000 mg daily, most preferably 2000 mg daily, provided that the second dose is higher than the first dose.

According to an embodiment, the second period is up to 16 weeks, preferably 2 to 12 weeks, more preferably 3-10 weeks, even more preferably 4-8 weeks, most preferably 4 weeks. Alternatively, the second period is up to 4 months, preferably 0.5 to 3 months, more preferably 1.5-2.5 months, even more preferably 1-2 months, most preferably 1 month.

According to an embodiment, the third dose is ≥1500 mg daily, preferably 2000 to 5000 mg daily, more preferably 2000 to 3000 mg or 3000 to 4000 mg daily, most preferably 3000 mg daily, provided that the third dose is higher than the first and second dose.

According to an embodiment, the third period is up to 16 weeks, preferably 2 to 12 weeks, more preferably 3-10 weeks, even more preferably 4-8 weeks, most preferably 4 weeks. Alternatively, the third period is up to 4 months, preferably 0.5 to 3 months, more preferably 1.5-2.5 months, even more preferably 1-2 months, most preferably 1 month.

In the above-described method steps, the following sequence of steps is referred to as “escalation cycle”: administering NR to the subject at a certain dose over a certain period until timepoint Vₙ; measuring one or more biological parameter(s) at timepoint Vₙ, wherein: if a biological response is achieved, the certain dose is maintained, and if a biological response is not achieved, the dose is increased to a further dose.

Optionally, in the above-described method, instead of maintaining the last escalation dose or discontinuing the treatment, one or more further escalation cycles may be additionally performed. In the above-described method steps, optionally one of the recited escalation cycles can be omitted, e.g., after the first cycle, the third cycle may follow directly.

According to an embodiment, the maintenance dose of NR determined and administered according to the method of the invention is >1000, ≥2000, >2000, ≥3000 >3000, ≥4000 or ≥5000 mg daily, preferably 2000 to 5000 mg daily, more preferably 2000 to 3000 mg or 3000 to 4000 mg daily, most preferably 3000 mg daily. NR is well tolerated with no evidence of toxicity in adult humans with doses up to at least 2000 mg daily. Without wishing to be bound by theory, we consider than oral administration of the NAD precursor NR in dosages of up even higher dosages, when accompanied by the particular measurement scheme according to the present invention, is unlikely to cause moderate or severe side effects as measured biochemically and physiologically, and that it is unlikely to have significant tolerability issues for treated individuals.

According to an embodiment, the biological response is or comprises increase of cerebral NAD levels. NAD is the central redox coenzyme in cellular metabolism and critical supplier of energy equivalents to the respiratory chain. By regulating the activity of the deacetylase enzymes known as sirtuins, NAD regulates several fundamental cellular events including histone acetylation and, by extension, gene expression. The signaling turnover of NAD in human cells is strikingly high with the entire cellular NAD pool being renewed at least once a day. This rapid turnover means that decreased NAD synthesis, as our findings suggest occurs in the PD brain, can have a profound and immediate impact on neuronal metabolism, including ATP deficiency, altered gene expression and compromised neuronal function and survival. The present inventors believe that increase of cerebral/neuronal NAD can improve mitochondrial function, restore sirtuin activity and histone acetylation status and rescue neuronal dysfunction and death in PD. Surprisingly, nicotinamide riboside for use according to the present invention is capable of passing the blood-brain barrier, and effectively increase the cerebral NAD levels.

According to an embodiment, the biological response is or comprises increase of cerebral NAD levels. Such increase may be, e.g., in the occipital cortex. The cerebral NAD levels can be measured by ³¹Phosphorus magnetic resonance spectroscopy (³¹P-MRS), e.g., by determining a cerebral NAD/ATP-α molar ratio. The biological response can be or comprise increase of cerebral NAD levels by ≥10%, preferably ≥30%, more preferably ≥50%; e.g., increase of cerebral NAD/ATP-α molar ratio by ≥10%, preferably ≥30%, more preferably ≥50%. Alternatively or additionally, the biological response can be or comprise absolute increase in the cerebral NAD/α-ATP-α molar ratio is ≥0.01, preferably ≥0.03, more preferably ≥0.07, most preferably ≥0.01.

Cerebral NAD levels, such as NAD/α-ATP-α molar ratios, can be calculated by using ³¹P spectroscopy data from magnetic resonance imaging and spectroscopy (MRI/S) e.g., as shown in FIG. 1B. MRI/S can be conducted on a 3T MR-PET scanner (e.g., Biograph mMR Siemens Healthcare, Germany); Phosphorous MRS (³¹P-MRS) can be performed on a double-resonant transmit/receive ¹H/³¹P volume head-coil (e.g., Rapid Biomedical, Germany). For instance, an anatomical T1-weighted image with the following sequence parameters can be acquired to aid positioning of the ³¹P-imaging slab: MPRAGE 3D T1-weighted sagittal volume, TE/TR/TI=2.26 ms/2.4 s/900 ms, acquisition matrix=256×256×192, FOV=256×256×192 mm3, 200 Hz/px readout bandwidth, flip angle=8 degrees and total acquisition duration of 5.6 minutes. ³¹P spectroscopy data can then be acquired using a 3D chemical shift imaging (CSI) FID sequence with WALTZ4 1H decoupling and continuous wave nuclear Overhauser effect (NOE) enhancement (Peeters et al., NMR Biomed. 2019; 34: e4169). A CSI grid with an 8×8 matrix and nominal voxel size of 30×30×80 mm³, 1024 samples, readout length=512 ms, 1000 Hz bandwidth, field of view (FOV)=240×240×80 mm³, TE/TR=2.3 ms/3.0 s, 10 averages, flip angle=90 degrees and total acquisition duration of 14.5 minutes, are used. Rectangular NOE pulses of 10 ms length, interpulse delay 1 ms, train length 10 prior and WALTZ4 decoupling (2 ms pulses, 180 deg. flip angle) are applied prior to ³¹P-excitation and during the first half of the acquisition window respectively. The FOV is centered on the brain midline and aligned parallel to the anterior and posterior commissure. Spectra from the occipital region are aligned using an adaption of the Spectral Registration implementation from Gannet 3.0, subject to thresholding on SNR (>=3) to eliminate the majority of out-of-brain voxels. Voxels are averaged before being processed in Matlab 9.5 (the MathWorks, Natick, MA) using the OXSA toolbox (Purvis et al., PLoS One 2017; 12: e0185356) utilizing first order phase correction and fitting with AMARES. Custom prior information may be created based on literature values for membrane phospholipids (MP), glycerophosphocholine (GPC), glycerophosphoethanolamine (GPE), inorganic phosphate (Pi), phosphocoline (PC), phosphoethanolamine (PE) as well as alpha-, beta- and gamma resonances of adenosine triphosphate (ATP-α, -β, and -γ, respectively) in reference to the phosphocreatine (PCr) peak (Deelchand D. K. et al, NMR Biomed. 2015; 28: 633-641; Peeters T. H. et al., NMR Biomed. 2019; 34: e4169; Ren et al., Magn. Reson. Med. 2018; 80: 1289-1297). Additional information for the properties of nicotinamide adenine dinucleotide (NAD) may be added based on the framework developed by Lu M. et al. (Magn. Reson. Med. 2014; 71: 1959-19) by calculating field-strength dependent chemical shift differences, relative amplitudes and frequency separations for oxidized and reduced NAD (NAD⁺ and NADH, respectively). Linewidths are fixed to be equal for NAD⁺, NADH and ATP-α. At 3T, and to comply with normal-mode specific absorption rate (SAR) restrictions, peak separation for NAD⁺ and NADH is limited and therefore only combined values of total NAD (NAD⁺ and NADH together) are preferably used.

According to an embodiment, the biological response is or comprises increase of NAD and/or related metabolite levels in CSF. These can be measured by HPLC-MS metabolomics or the NADmed method (https://www.nadmed.fi/).

HPLC-MS metabolomics for detecting NAD and/or related metabolite levels are described in Pirinen E, et al., Cell Metab. 2020 Jun. 2; 31(6):1078-1090.e5. doi: 10.1016/j.cmet.2020.04.008. Epub 2020 May 7. PMID: 32386566. Quantification of NR, NAM, niacin, MeNAM, NAM oxide, Me2PY and Me4PY: as described under “Targeted Quantitative Metabolomics Analyses” therein; and/or Trammell, S. A., & Brenner, C. (2013). Targeted, LCMS-based Metabolomics for Quantitative Measurement of NAD⁺Metabolites. Computational and Structural Biotechnology Journal, Volume No: 4, Issue: 5, January 2013, e201301012, http://dx.doi.org/10.5936/csU.201301012; and/or Trammell, S. A., et al. (2016). Scientific reports, 6, 26933. https://doi.or/10.1038/sre 26933; and/or Elhassan, Y. S., et al. (2019). Cell reports, 28(7), 1717-1728.e6. https://doi.org/10.1016/j.celrep.2019.07.043). The NADmed method (https://www.nadmed.fi/) is described, for instance, in WO 2022/008802 A1, e.g., Example 1 therein. It involves, inter alia, obtaining an extract comprising by contacting the sample with pre-heated to at least 40° C. (e.g., 70° C.) alcohol (e.g., methanol) solution, and a cycling assay involving a cyclic enzymatic reaction (with an enzyme which specifically recognizes and uses NAD⁺ as substrate) with photometric (colorimetric) detection.

Preferably, the levels of one or more metabolite(s) of the NAD-metabolome are measured; accordingly, the biological response is or comprises increase in the CSF levels of one or more metabolite(s) of the NAD-metabolome. Specific metabolites include one or more selected from: nicotinic acid, nicotinic acid riboside, nicotinamide, nicotinamide riboside, nicotinic acid mononucleotide, nicotinamide mononucleotide, nicotinic acid adenine dinucleotide, nicotinamide adenine dinucleotide, methyl nicotinamide, nicotinamide N-oxide, N-methyl-2-pyridone-5-carboxamide, and N-methyl-4-pyridone-5-carboxamide.

According to an embodiment, the biological response is or comprises increase of blood NAD levels. These can be measured by HPLC-MS metabolomics, or by the NADmed method (see references above).

According to an embodiment, the biological response is or comprises increase in NRRP expression (e.g., the NRRP score). This can be measured by 18F-fluorodeoxyglucose positron emission tomography (FDG-PET). Additionally, an increase in the NRRP subject score is considered to correlate with the decrease in the UPDRS score.

The NRRP expression can be derived from the FDG-PET data, as previously described (Cell Metab. 2022 Mar. 1; 34(3):396-407.e6). For example, a NR-related metabolic pattern (NRRP) can be identified by using ordinal trends/canonical variates analysis (OrT/CVA), a supervised form of principal component analysis (PCA) (Habeck et al., (2005), Neural Computation 17, 1602-1645). The significance of the resulting OrT/CVA topographies can be assessed using nonparametric tests, i.e., permutation testing of the subject scores to show that the observed ordinal trend did not occur by chance. Likewise, the reliability of the voxel loadings (i.e., region weights) on the resulting network topography can be assessed using bootstrap resampling procedures (Habeck and Stern (2010), Cell Biochemistry and Biophysics 58, 53-67; Mure et al. (2011), Neuroimage 54, 1244-1253).

TABLE 1
FDG-PET; Brain regions with reduced metabolic
activity as part of the NRRP network

| | | MNI coordinates* | | |
|---|---|---|---|---|
| Brain region | Brodmann area | x | y | z |
| Lentiform Nucleus (left) | Putamen | −20 | 10 | −8 |
| | | −28 | −2 | −2 |
| Lentiform Nucleus (right) | Putamen | 22 | 10 | −8 |
| Thalamus (left) | Anterior | −10 | −22 | 4 |
| Thalamus (right) | Medial Dorsal | 8 | −18 | 4 |
| Anterior Cingulate (left) | 32 | −4 | 44 | 18 |
| Anterior Cingulate (right) | 32 | 4 | 46 | −8 |
| | 24 | 4 | 8 | 48 |
| Posterior Cingulate (bilateral) | 31 | 0 | −30 | 36 |
| Precuneus (left) | 7 | −6 | −72 | 40 |
| Middle Frontal Gyrus (left) | 8 | −26 | 30 | 42 |
| Superior Frontal Gyrus (left) | 9 | −32 | 44 | 28 |
| | 10 | −28 | 56 | 6 |
| Prefrontal Cortex (right) | 9 | 44 | 16 | 30 |

*Montreal Neurological Institute standard space.

The analysis can be restricted to the top PC patterns, accounting for greater than 75% of the subject×voxel variance in the longitudinal data. Expression values for these PCs can then be entered singly and in all possible linear combinations to identify significant monotonic trends in the individual subject data, i.e., consistent increases (or decreases) in pattern expression across the subjects with few if any violations (p<0.05; permutation test, 1000 iterations). The resulting coefficients are applied to the corresponding PC patterns to construct the NR-related topography. For the NRRP to be significant, voxel weights preferably must have low dispersion (inverse coefficient of variation (ICV) |z|>1.96, p<0.05; bootstrap resampling (1,000 iterations), indicating that regional loadings are not driven by outliers). The analysis can be performed within a population or the individual subject's gray matter brain mask defined by the FDG-PET scans, e.g., as outlined in Table 1.

According to an embodiment, the treatment involves bilateral metabolic reductions in the caudate and putamen, extending into the adjacent globus pallidus, and in the thalamus, as determined by FDG-PET. Preferably, these changes are also associated with localized cortical reductions along the medial wall of the hemisphere involving the precuneus (BA 7), medial frontal cortex (BA 9, 10), anterior cingulate area (BA 24, 32), and in the posterior cingulate gyrus (BA 31).

According to an embodiment NR for use in the method for threating PD is provided, wherein:
the first dose is 1000 mg daily;
a 31P-MRS scan is conducted after 30 days at timepoint V2, wherein if a cerebral NAD response is achieved at V2, treatment is continued on this maintenance dose, and if a cerebral NAD response is not achieved at V2, the dose is increased to 1500 mg;
a new 31P-MRS scan is conducted after 30 days at timepoint V3, wherein if a cerebral NAD response is achieved at V3, treatment is continued on this maintenance dose, and if a cerebral NAD response is not achieved at V3, the dose is increased to 2000 mg, preferably 1000 mg twice daily;
a new 31P-MRS scan is conducted after 30 days at timepoint V4, wherein if a cerebral NAD response is achieved at that point V4, treatment is continued on this on this dose, and if a cerebral NAD response is still not achieved, the treatment is discontinued by gradual tapering, preferably 1500 mg daily for one week, then 1000 mg daily for one week, then stop of NR intake.

According to an embodiment NR for use in the method for threating PD is provided, wherein:
the first dose is 1000 mg daily;
a 31P-MRS scan is conducted after 30 days at timepoint V2, wherein if a cerebral NAD response is achieved at V2, treatment is continued on this maintenance dose, and if a cerebral NAD response is not achieved at V2, the dose is increased to 2000 mg;
a new 31P-MRS scan is conducted after 30 days at timepoint V3, wherein if a cerebral NAD response is achieved at V3, treatment is continued on this maintenance dose, and if a cerebral NAD response is not achieved at V3, the dose is increased to 3000 mg, preferably 1500 mg twice daily;
a new 31P-MRS scan is conducted after 30 days at timepoint V4, wherein if a cerebral NAD response is achieved at that point V4, treatment is continued on this on this dose, and if a cerebral NAD response is still not achieved, the treatment is discontinued by gradual tapering of 1000 mg every week, preferably 2000 mg daily for one week, then 1000 mg daily for one week, then stop of NR intake.

In a second aspect, the present invention provides NR for use in a method for treating Parkinson's Disease (PD) in a human subject at a dose of more than 3000 mg daily. The dose of NR can be more than 3000 mg daily; preferably more than 3000 mg and up to 5000 mg daily; more preferably: more than 3000 mg and up to 4000 mg daily, or 4000 to 5000 mg daily. Subjects who may particularly benefit from such dosages can be identified, e.g., by applying the dose escalation/measurement protocol of the method used in the present invention.

According to an embodiment, the treatment is safe and/or tolerable. Safety can be assessed, for instance, by the absence, or substantial absence, of moderate or severe adverse events, preferably having a probable or definite causal relationship to NR and/or being clinically deemed to compromise patient safety. Tolerability can be assessed by the absence, or substantial absence, of mild or moderate adverse events, preferably having a definite causal relationship to NR and/or decreasing treatment compliance. Severity is categorized as mild, moderate or severe, and causal relationship of an event to the treatment is categorized as unrelated, unlikely, possible, probable or definite, as further defined in Example 1.

Safety and/or tolerability may also be assessed by change(s) or deviation(s) from reference values in vital signs and/or clinical laboratory values. The vital signs are preferably selected from blood pressure, pulse, and body weight. The clinical laboratory values are preferably selected from CRP, ALAT, ASAT, GT, Bilirubin, ALP, Creatinine, Urea, RBC, Hb, WBC with differential, Platelets, CK, FT4, TSH, B12, Folic acid, homocysteine, Methylmalonic acid, Sodium, and Potassium.

According to an embodiment, the treatment involves improving clinical dysfunction in PD; and/or improving motor, non-motor and/or cognitive symptoms; or preventing motor, non-motor and/or cognitive symptoms. Preferably, the improvement or prevention is assessed by change(s) in one or more of: a) total MDS-UPDRS and/or each subsection I. 11 or III of the MDS-UPDRS questionnaire; preferably by at least 1 or at least 2 points; b) NMSQ total score, assessed by the NMSQ questionnaire; c) NMSS total score, assessed by the NMSS questionnaire; d) MoCA total score, assessed by the MoCA questionnaire; e) EQ-5L score. Assessed by the EQ-5L questionnaire; and/or f) Hoehn & Yahr Stage, assessed by the Hoehn & Yahr stage in MDS-UPDRS.

According to an embodiment, the total score of parts I, II, III and IV, or the total score of parts I, II and III according to the Movement Disorder Society Revision of the Unified Parkinson's Disease Rating Scale (MDS-UPDRS; see above) is improved. The MDS-UPDRS has four parts: Part I (non-motor experiences of daily living), Part II (motor experiences of daily living), Part III (motor examination) and Part IV (motor complications). Preferably, the improvement is an improvement in the mean difference in the total score after the treatment as compared to baseline before the treatment. Preferably, the improvement in the total MDS-UPDRS score according to parts I,-IV; or the total MDS-UPDRS score according to parts I-III; or any of the individual parts, is at least 1 point, more preferably 2 points.

Preferably, the improved or prevented motor, non-motor and/or cognitive symptoms are one or more of the symptoms/criteria according to the MDS-UPDRS:
Part I—cognitive impairment; hallucinations and psychosis; depressed mood; anxious mood; apathy; features of dopamine dysregulation syndrome (DDS); sleep problems; daytime sleepiness; pain and other sensations; urinary problems; constipation problems; light headedness on standing; fatigue;
Part II—speech; saliva and drooling; chewing and swallowing; eating tasks; dressing; hygiene; handwriting; doing hobbies and other activities; turning in bed; tremor; getting out of bed; walking and balance; freezing; is the patient on medication?; patient's clinical state (OFF: typical functional state when patients have a poor response in spite of taking mediation or the typical functional response when patients are on no treatment for parkinsonism; ON: typical functional state when patients are receiving medication and have a good response); is the patient on levodopa?; if yes, minutes since last dose;
Part III—speech; facial expression; rigidity—neck; rigidity—RUE; rigidity—LUE; rigidity—RLE; rigidity—LLE; finger tapping—right hand; finger tapping—left hand; hand movements—right hand; hand movements—left hand; pronation—supination movements—right hand; pronation—supination movements—left hand; toe tapping—right foot; toe tapping—left foot; leg agility—right leg; leg agility—left leg; arising from chair; gait; freezing of gait; postural stability; posture; global spontaneity of movement; postural tremor—right hand; postural tremor—left hand; kinetic tremor—right hand; kinetic tremor—left hand; Rest tremor amplitude—RUE; Rest tremor amplitude—LUE; Rest tremor amplitude—RLE; Rest tremor amplitude—LLE; rest tremor amplitude—lip/jaw; constancy of rest tremor; were dyskinesias present?; did these movements interfere with ratings?; Hoehn and Yahr Stage;
Part IV—time spent with dyskinesias; functional impact of dyskinesias; time spent in the OFF state; functional impact of fluctuations; complexity of motor fluctuations; painful OFF-state dystonia.

Preferably, the improved or prevented motor symptoms are one or more of the symptoms/criteria according to more of the symptoms/criteria according to Parts II, III and IV of MDS-UPDRS. Preferably, the non-motor and/or cognitive symptoms are one or more of the symptoms/criteria according to Part I MDS-UPDRS.

Preferably, the improved or prevented non-motor symptoms are one or more of the symptoms/criteria according to the Non-Motor Symptoms Questionnaire (NMSQ: PD NMS QUESTIONNAIRE developed and validated by the International PD Non MotorGroup (2006), International Parkinson and Movement Disorder Society, https://www.movementdisorders.org/MDS-Filesl/Education/Rating-Scales/NMSQ.pd): Dribbling of saliva during the daytime; Loss or change in ability to taste or smell; Difficulty swallowing food or drink or problems with choking; Vomiting or feelings of sickness (nausea); Constipation (less than 3 bowel movements a week) or having to strain to pass a stool (faeces); Bowel (fecal) incontinence; Feeling that bowel emptying is incomplete after having been to the toilet; A sense of urgency to pass urine makes the subject rush to the toilet; getting up regularly at night to pass urine; Unexplained pains (not due to known conditions such as arthritis); Unexplained change in weight (not due to change in diet); Problems remembering things that have happened recently or forgetting to do things; Loss of interest in what is happening around the subject or doing things; Seeing or hearing things that the subject knows or is told are not there; Difficulty concentrating or staying focused; Feeling sad, ‘low’ or ‘blue’; Feeling anxious, frightened or panicky; Feeling less interested in sex or more interested in sex; Finding it difficult to have sex when you try; Feeling light headed, dizzy or weak standing; from sitting or lying; Falling; Finding it difficult to stay awake during activities, such as working, driving or eating; Difficulty getting to sleep at night or staying asleep at night; Intense, vivid dreams or frightening dreams; Talking or moving about in your sleep as if the subject is ‘acting’ out a dream; Unpleasant sensations in your legs at night or; while resting, and a feeling that you need to move; Swelling of the subject's legs. Excessive sweating; Double vision; believing things are happening to the subject that other people say are not true).

Preferably, the improved or prevented non-motor symptoms are one or more of the symptoms/criteria according to the Non-Motor Symptoms Scale for Parkinson's Disease (NMSS: Non-Motor Symptom assessment scale for Parkinson's Disease developed by the International Parkinson's Disease Non-Motor Group (2007). International Parkinson and Movement Disorder Society, (https://www.movementdisorders.org/MDS-Filesl/PDFs/Rating-Scales/NMSS.pd):
Domain 1: Cardiovascular including falls—light-headedness, dizziness, weakness on standing from sitting or lying position; falling because of fainting or blacking out;
Domain 2: Sleep/fatigue—dozing off or fall asleep unintentionally during daytime activities; fatigue (tiredness) or lack of energy (not slowness) limit the patient's daytime activities; having difficulties falling or staying asleep; an urge to move the legs or restlessness in legs that improves with movement when he/she is sitting or lying down inactive; Domain 3: Mood/Cognition—loss of interest in his/her surroundings; loss of interest in doing things or lack motivation to start new activities; feeling nervous, worried or frightened for no apparent reason; seeming sad or depressed or has he/she reported such feelings; having flat moods without the normal “highs” and “lows”; having difficulty in experiencing pleasure from their usual activities or report that they lack pleasure;
Domain 4: Perceptual problems/hallucinations—the subject indicates that he/she sees things that are not there; the subject having beliefs that he/she know are not true; double vision (2 separate real objects and not blurred vision);
Domain 5: Attention/Memory—having problems sustaining concentration during activities; forgetting things that he/she has been told a short time ago or events that happened in the last few days; forgetting to do things;
Domain 6: Gastrointestinal tract—dribbling saliva during the day; having difficulty swallowing; suffering from constipation (Bowel action less than three times weekly);
Domain 7: Urinary—having difficulty holding urine (Urgency); having to void within 2 hours of last voiding (Frequency); having to get up regularly at night to pass urine (Nocturia);
Domain 8: Sexual function—having altered interest in sex; (Very much increased or decreased, please underline); having problems having sex;
Domain 9: Miscellaneous—suffering from pain not explained by other known conditions; reporting a change in ability to taste or smell; reporting a recent change in weight (not related to dieting); excessive sweating (not related to hot weather).

Preferably, the score improvement on the Hoehn & Yahr scale is assessed by the by the Hoehn & Yahr stage as given in MDS-UPDRS, i.e.: 0: asymptomatic, 1: unilateral involvement only, 2: bilateral involvement without impairment of balance, 3: mild to moderate involvement; some postural instability but physically independent; needs assistance to recover from pull test, 4: severe disability; still able to walk or stand unassisted, 5: wheelchair bound or bedridden unless aided.

More preferably, the improved or prevented non-motor symptoms are one or more of the symptoms/criteria according to the MDS Non-Motor Rating Scale (MDS-NMS, Ray Chaudhuri, Anette Schrag, Daniel Weintraub, Alexandra Rizos, Carmen Rodriguez-Blazquez, Eugenia Mamikonyan and Pablo Martinez-Martin (2019): The International Parkinson and Movement Disorder Society—Non-Motor Rating Scale, https://www.movementdisorders.org/MDS-Filesl/PDFs/Rating-Scales/MDS-NMS_FINAL.pdf accessed on Jun. 22, 2021, and incorporated herein in its entirety by reference). Yet more preferably, the improvement or prevention of non-motor symptoms refers to improving or preventing deterioration in the MDS-NMS non-motor fluctuations total score.

Preferably, the cognitive symptoms are one or more of the symptoms/criteria according to the Montreal Cognitive Assessment (MoCA; Z. Nasreddine M D Version Nov. 7, 2004, https://www.parkinsons.va.gov/resources/MOCA-Test-English.pdf accessed on Jun. 22, 2021, and incorporated herein in its entirety by reference).

Preferably, the subject's quality of life assessed according to the EQ-5D-5L questionnaire is improved. EQ-5D-5L is the five-level version of EQ-5D, as described by Herdman M, Gudex C, Lloyd A, et al. Qual Life Res. 2011; 20(10):1727-1736. Preferably, the Sample UK English EQ-5D-5L is used (https://euroqol.org/eq-5d-instruments/sample-demo/, accessed on Jun. 22, 2021: https://euroqol.org/wp-content/uploads/2020/09/Sample_UK-English-EQ-5D-5L-Paper-Self-Complete-v1.2-ID-24700.pdf, incorporated herein in its entirety by reference).

According to an embodiment, the treatment involves altering the NAD metabolome in peripheral blood cells and/or CSF. This can assessed by change(s) in levels of NAD metabolites in PBMC and/or CSF, measured by HPLC-MS and/or the NADmed method.

According to an embodiment, the treatment does not involve altering methylation metabolism, in particular, decreased availability of methylation substrates, such as decreased availability of methyl-donors, such as SAM, decreased DNA methylation, such as globally or at specific sites, decreased synthesis of neurotransmitters like dopamine and serotonin, aberrant folate and one-carbon metabolism. This can be assessed by change(s) in levels of one-carbon metabolism metabolites, measured by: HPLC-MS metabolomics in PBMC and CSF; levels of monoamine neurotransmitters in CSF; or levels and/or genomic distribution of DNA methylation.

In an alternative embodiment, the treatment involves altering methylation metabolism, in particular, decreased availability of methylation substrates, such as decreased availability of methyl-donors, such as SAM, decreased DNA methylation, such as globally or at specific sites, decreased synthesis of neurotransmitters like dopamine and serotonin, aberrant folate and one-carbon metabolism.

The subject may have or not have, but preferably does not have p.A222V (also referred to as c.677C>T), p.E429A (also referred to as c.1298A>C), and/or other variant(s) of a MTHFR gene which decrease the activity of the enzyme methylenetetrahydrofolate reductase (MTHFR). Methylenetetrahydrofolate reductase (UniProt: P42898 (MTHR_HUMAN); canonical sequence identifier: P42898-1; HGNC:7436, MTHFR), encoded by the MTHFR gene, is essential for one-carbon metabolism. MTHFR catalyzes the irreversible conversion of 5,10-methylenetetrahydrofolate (5,10-methylTHF) to 5-methylTHF, which serves as methyl donor for the synthesis of methionine from homocysteine. Methionine is then converted to S-adenosyl-L-methionine (SAM), which is a universal methyl-group donor for a variety of methylation reactions, including DNA-methylation, neurotransmitter synthesis, etc. The canonical sequence of MTHFR is shown in SEQ ID NO. 6.

Genetic variation in the MTHFR gene is associated with folate deficiency, elevated homocysteine levels and lower availability of methyl groups for methylation reactions. Furthermore, individuals carrying such mutations have been shown to have significantly lower levels of 5-methylcytosine in genomic DNA under conditions of low folate status (Friso S, Choi S W, Girelli D, et al. PNAS 2002; 99(8):5606-5611).

Thus, it stands to reason that individuals who are homozygous (and optionally heterozygous carriers) for certain MTHFR variants would be substantially more susceptible to NR-induced methylation depletion. This is important due to the high population frequency of MTHFR variation. The two most common variants that decrease the activity of the MTHFR enzyme are the p.A222V (often referred to as c.677C>T), and the p.E429A (often referred to as c.1298A>C) variants. Both are inherited as AR. These two variants are in linkage disequilibrium with each other, making compound heterozygosity an infrequent occurrence. The p.A222V has a global allele frequency of 0.30 (0.34 in non-Finnish Europeans and 0.50 in Latino/Admixed Americans), and ˜11% of the population is homozygous. The p.E429A has a global allele frequency of 0.29 (0.32 in non-Finnish Europeans and 0.41 in South Asians), and ˜9% of the population is homozygous (Data derived from gnomAD: Karczewski K J, Francioli L C, Tiao G, et al. Nature 2020; 581(7809):434-443).

p.A222V (also referred to as c.677C>T) and p.E429A (also referred to as c.1298A>C), are variants of the MTHFR gene which decrease the activity of the enzyme methylenetetrahydrofolate reductase (MTHFR). The presence of such mutations can be detected in a DNA sample, e.g., from whole blood, muscle biopsy tissue and/or PBMC homogenate, e.g., by Sanger sequencing, SNP-Chip, rtPCR, or other methods known in the art.

Individuals who are carriers, (e.g., heterozygous or homozygous, preferably homozygous carriers) for rare pathogenic mutations and/or common variants MTHFR gene which decrease the activity of the MTHFR enzyme, are potentially susceptible to NR-induced methylation depletion.

In view of the above, the treatment may be supplemented by concomitant administration of one or more methyl donor(s), e.g., if the treatment involves altering methylation metabolism and/or the subject caries variant(s) of a MTHFR gene which decrease the activity of the enzyme methylenetetrahydrofolate reductase. The term “methyl donor(s)” refers to methylated substances having biologically labile methyl groups which are easily passed to other molecules through a transmethylation reaction, or any other compounds capable of increasing the availability of methyl groups (methyl equivalents) upon administration to a human subject. a) S-adenosyl methionine, preferably at a dose of 400 to 600 mg daily; b) 5-methyltetrahydrafolate, preferably at a daily dose of 0.5-30 mg, more preferably 5-15 mg 5-methyltetrahydrafolate, even more preferably 1-15 mg, or 3-10 mg, or 5-10 mg, most preferably 1-3 mg, 5-10 mg, or 12-18 mg L-methyltetrahydrofolate; c) folic acid, preferably at a dose of 200 to 600 mg, more preferably about 400 mg daily d) Vitamin B12, preferably cyanocobalamine or methylcobalamine, more preferably methylcobalamine, preferably at a dose of 1-1000, preferably 2-500, more preferably 2-25 μg daily; e) Vitamin B6, preferably pyridoxine, and preferably at a dose of 0.5-4, preferably 1-2 mg daily; f) methionine, preferably at dose of 500-2500 mg, more preferably 1500-200 mg daily; g) choline or choline source(s), preferably i. glycerophosphocholine; ii. phosphocholine; iii. phosphatidylcholine; iv. lysophosphatidylcholine; v. sphingomyelin; and/or vi. choline; h) L-carnitine, preferably at a dose of 500-2,000 mg daily; i) curcumin, preferably at a dose of 150-250 mg daily; j) quercetin, preferably at a dose of 500-1000 mg daily; k) zinc source(s), preferably zinc salt(s), more preferably zinc acetate, chloride, citrate, gluconate, lactate, oxide, carbonate or sulfate, and preferably at a daily dose 25-400, more preferably 50-200 mg zinc daily; l) betaine, preferably at a daily dose of 40 mg-10 g, preferably 1.8-5.4 g, more preferably 40-700 mg, more preferably 42.5-340 mg, most preferably 42.5-85 mg betaine (e.g., trimethylglycine); m) riboflavin, Vitamin B6, 5-methyltetrahydrafolate, Vitamin B12, and/or betaine, one of more which, preferably all of which, are present in the same composition; preferably at a daily dose of 45-550 mg, more preferably 90-270 mg riboflavin (e.g., as riboflavin 5′-phosphate sodium); 20-200 mg, more preferably 45-135 mg Vitamin B6 (e.g., as pyridoxal 5′-phosphate); 1-30 mg, more preferably 3-12 mg L-5-methyltetrahydrafolate (e.g., 5-15 mg in the form of L-methyltetrahydrofolic acid, glucosamine salt); 1.5-18 mg, more preferably 3-9 mg Vitamin B12 (e.g., as methylcobalamin); and/or 40 mg-10 g, more preferably 1.8-5.4 g betaine (e.g., trimethylglycine); n) betaine, quercetin, resveratrol, and/or betaine, one of more which, preferably all of which, are present in the same composition; preferably at a daily dose of 40-700 mg, more preferably 85-340 mg betaine (e.g., trimethylglycine); 125-2000 mg, more preferably 250-1000 mg quercetin (e.g., as quercetin phytosome, or Sophorajaponica extract (flower)/phospholipid complex from sunflower); and/or 75-1200, more preferably 150-600 mg resveratrol (e.g., trans-resveratrol), o) a composition comprising: i) serine and/or glycine, preferably serine, and/or preferably in a dosage of in a dose of 0.48-24 mmol/kg/day, such as 0.48-4.8 mmol/kg/day, such as 1.8-4.8 mmol/kg/day, such as 2.9-4.6 mmol/kg/day; ii) N-acetyl cysteine, cysteine and/or cystine, preferably N-acetyl cysteine and/or preferably in a dose of 0.31-3.05 mmol/kg/day, such as 0.31-1.84 mmol/kg/day, such as 0.43-1.23 mmol/kg/day; ii) optionally carnitine, deoxycarnitine, gamma-butyrobetaine, 4-trimethylammoniobutanal, 3-hydroxy-N6,N6,N6-trimethyl-L-lysine, N6,N6,N6-trimethyl-L-lysine and/or lysine, preferably carnitine and/or preferably in a dose of 0.031-1.24 mmol/kg/day, such as 0.031-0.620 mmol/kg/day, such as 0.062-0.50 mmol/kg/day, such as 0.093-0.37 mmol/kg/day; p) an aqueous solution or suspension comprising: i) serine; ii) N-acetyl cysteine; and ii) carnitine, preferably wherein the molar ratio of i) to ii) is between 12:1 and 1:1.5, more preferably between 10:1 and 3:1, the molar ratio of i) to iii) is between 100:1 and 4:1, more preferably between 50:1 and 8:1, even more preferably between 30:1 and 13:1; or q) a combination of two or more thereof.

According to an embodiment, the treatment involves ameliorating proteostasis. This can assessed by change(s) in gene and protein expression levels of factors involved in lysosomal and proteasomal function.

According to an embodiment, the treatment involves altering histone acetylation status. This can be assessed by change(s) in the levels of histone panacetylation, and levels and genomic distribution of H3K27 and H4K16 acetylation in PBMC, measured by immunoblotting and chromatin immunoprecipitation sequencing (ChIPseq). Genome-wide aberrant histone hyperacetylation and altered transcriptional regulation occur in the brain of individuals with PD. Preferably, the subject's aberrant histone acetylation is characterized by increased acetylation of multiple sites on histones H2B, H3 and H4, more preferably, H3K27 hyperacetylation (H3K27ac) as compared to a control subject not suffering from PD. Preferably, the aberrant gene expression profile is characterised by increased levels of SIRT1 and SIRT3 proteins in the brain as compared to a control subject not suffering from PD. Without wishing to be bound by theory, it is possible that NR may mitigate epigenomic dysregulation in PD, by regulating histone acetylation. Increasing neuronal NAD levels may boost the activity of the NAD-dependent histone deacetylases of the sirtuin family, potentially ameliorating histone hyperacetylation in PD.

According to an embodiment, the treatment involves decreasing neuroinflammation. This can be assessed by change(s) in the levels of inflammatory cytokines in serum and/or CSF measured using ELISA.

According to an embodiment, the treatment involves influencing the gene and/or protein expression levels in PBMC. This can be assessed by change(s) in RNA sequencing (RNAseq) and LC-MS proteomics, respectively.

According to an embodiment, the treatment involves influencing the gut microbiome in PD. This can be assessed by change(s) in metagenomics in fecal samples.

In any of the above embodiments, said change(s) or absence can refer to an overall (global) change, e.g., change relative to placebo, or overall (global) absence; and/or a specific (individual) change concerning a given point in time or period during treatment, e.g., change relative to an earlier point in time or period during treatment or before the treatment, or absence for a specific (individual) subject. As used herein, “substantial absence” may refer to frequency and/or severity of events not statistically significantly different between a treatment population, and a placebo or untreated population.

According to an embodiment, the treatment is for preventing, decreasing and/or delaying the progression of PD.

According to an embodiment, the treatment is a neuroprotective therapy. In the context of the present invention, the term “neuroprotective therapy” refers to a treatment which protect neurons from degeneration (i.e., dysfunction and/or death), and enables their recovery and restoring of their functions; more specifically, any intervention which delays or prevents the death of dopaminergic neurons and other neuronal populations and/or cell types of the central, peripheral and/or autonomic nervous systems, which are affected in PD, and, therefore, slows or halts disease progression and/or prevents disease initiation. While dopaminergic neurons are a cell type affected in PD, PD affects also many other different neuronal populations across the central and autonomic/peripheral nervous systems. Thus, neuroprotection as used herein generally relates to preventing the death of dopaminergic neurons and/or any other neuronal population affected by PD across the central and autonomic/peripheral nervous systems.

According to an embodiment, the subject has nigrostriatal degeneration or denervation; and/or wherein the treatment delays nigrostriatal degeneration or denervation, and clinical disease progression. This can be confirmed by a positive [¹²³I]FP-CIT single photon emission CT (DaTscan).

According to an embodiment, the treatment acts as a neuroprotective, disease modifying therapy for PD dementia (PDD) and/or dementia with Lewy bodies (DLB). PDD and DLB are characterized by widespread neuronal death in both cortical and subcortical areas. The neuronal loss occurs and progresses over several years after the patient is diagnosed. There is, therefore, a substantial neuronal pool that could be rescued if the NR intervention starts as early as possible.

Based on the present findings, there is a good technical rationale that NR is suitable as neuroprotective, disease modifying therapy for PDD and DLB. Based on the present study results, as well as a body of preclinical evidence for NR-mediated neuroprotection, and without wishing to be bound by theory, it is considered that NR may increase neuronal resilience in the face of cellular stress, including but not limited to: mitochondrial respiratory dysfunction, free radical damage, aberrant lysosomal and/or proteasomal function, neuroinflammation, pathological protein aggregation such as a-synuclein, tau, TDP-43 and beta-amyloid. By increasing neuronal resilience and, therefore, survival, NR exerts a neuroprotective action, delaying and/or preventing the death of neurons. According to an embodiment, the subject is of age equal to or greater than 35 years at begin of treatment; preferably equal to or greater than 65 years, more preferably 35 to 85, more preferably 40 to 80, more preferably 55 to 75, more preferably 60 to 75, most preferably 65 to 75 years.

According to an embodiment, the PD is early PD.

In the context of the present invention, PD can be during its pre-motor or motor phase. PD during its pre-motor phase can refer to the time before the classic motor features of tremor, rigidity and bradykinesia become apparent.

Pre-motor phase PD can be divided into stages leading to manifest PD based on the presence of clinical, physiological or risk-markers of disease. Working backward from recognizable PD that could be diagnosed based on accepted criteria such as those by the UK Brain Bank, these stages include: 1) the pre-diagnostic phase, 2) the pre-motor phase, the 3) pre-clinical phase and 4) the pre-physiological phase. Diagnostic criteria and definitions for these phases are described in Siderowf, A. Mov Disord. 2012 April 15; 27(5): 608-616. According to a preferred embodiment, the PD is in the pre-diagnostic phase, pre-motor phase, pre-clinical phase or pre-physiological phase. Without wishing to be bound by theory, the patient to be treated is preferably in the early pre-motor or more preferably presymptomatic (pre-diagnostic) phases, from the viewpoint of achieving greater therapeutic success.

Alternatively, PD in the context of the present invention can be in Phase I (risk stage), Phase II (premotor stage), Phase III (early motor stage) and Phase IV (advanced PD), as defined in Bargiotas P, et al. Current Opinion in Neurology. 2016 Dec; 29(6):763-772. According to a preferred embodiment, the PD is in the Phase I (risk stage), Phase II (premotor stage) or Phase III (early motor stage).

PD affects ˜2% of everyone above 65 years and ˜4% of everyone above 85. Since NR is non-toxic, it can be beneficial to use NR as a prophylactic agent, e.g., in the general elderly population, preferably for subjects over age of 60 to prevent PD (i.e., even before PD is diagnosed or symptoms occur).

According to an embodiment, the subject is newly diagnosed with PD, preferably within 1, 2, 3, 4 or 5, more preferably within 2 years before begin of treatment. Additionally or alternatively, the subject's first PD symptom has been observed 50 months or less, preferably 45 months or less, more preferably 40 months or less, more preferably 30 months or less, more preferably 35 months or less, more preferably 25 months or less, more preferably 20 months or less, more preferably 15 months or less, more preferably 10 months or less, most preferably 5 months or less before begin of treatment.

According to an embodiment, the subject is drug naïve with respect to dopaminergic treatment prior to the treatment.

According to an embodiment, the subject at begin of treatment has an age of ≥35 years, preferably ≥40 years, more preferably ≥50 years, even more preferably ≥60 years, most preferably ≥65 years; or 35 to 85, preferably 40 to 80, more preferably 50 to 80, even more preferably 60 to 75, most preferably 65 to 75 years.

According to an embodiment, the PD is selected from idiopathic (IDP), juvenile; early-onset parkinsonism; secondary parkinsonism; atypical parkinsonism; vascular parkinsonism; drug-induced parkinsonism; multiple system atrophy (MSA); progressive supranuclear palsy; and/or monogenic PD. That is, PD can be, inter alia: idiopathic (IDP), juvenile (parkinsonism beginning during childhood or adolescence; early-onset parkinsonism (with onset between ages 21 and 40 years is sometimes called young or early-onset Parkinson disease); secondary parkinsonism (brain dysfunction that is characterized by basal ganglia dopaminergic blockade and that is similar to Parkinson disease, but it is caused by something other than Parkinson disease (e.g., drugs, cerebrovascular disease, trauma, postencephalitic changes)); atypical parkinsonism (a group of neurodegenerative disorders that have some features similar to those of Parkinson disease but have some different clinical features, a worse prognosis, a modest or no response to levodopa, and a different pathology (e.g., neurodegenerative disorders such as multiple system atrophy, progressive supranuclear palsy, dementia with Lewy bodies, and corticobasal ganglionic degeneration)); vascular parkinsonism (also known as arteriosclerotic parkinsonism, affecting people with restricted blood supply to the brain); drug-induced parkinsonism (parkinsonism caused by drugs, e.g., neuroleptic drugs (used to treat schizophrenia and other psychotic disorders), which block the action of the dopamine in the brain); multiple system atrophy (MSA); progressive supranuclear palsy (PSP, including Steele-Richardson-Olszewski syndrome); and/or monogenic PD/parkinsonism, for example caused by mutations in the genes: LRRK2, SNCA, PRKN, PINK1, DJ-1, VPS35.

For example, the subject having early PD may be diagnosed with PD within 1, 2, 3, 4 or 5, preferably 2 years or less before start of treatment. Diagnosis can be performed, for instance, according to the MDS clinical diagnostic criteria for Parkinson's disease, as explained above. Preferably, the subject's first PD symptom has been observed 50 months or less, preferably 45 months or less, more preferably 40 months or less, more preferably 30 months or less, more preferably 35 months or less, more preferably 25 months or less, more preferably 20 months or less, more preferably 15 months or less, more preferably 10 months or less, most preferably 5 months or less before begin of treatment.

Preferably, the subject has clinical diagnosis of idiopathic PD at begin of treatment, e.g., according to the MDS criteria, and/or the subject does not have pathogenic mutations in genes linked to monogenic PD, such as SNCA, LRRK2, VPS35, PRKN, and/or PINK1. For instance, the subjects intended to be treated can be genetically characterized using RNA-sequencing data from blood cells (PBMCs) and/or muscle.

Using this data, the patients can be assessed for known or novel established or potentially pathogenic mutations in all genes linked to monogenic PD (SNCA, LRRK2, VPS35, PRKN, PINK1). Canonical sequences of the respective proteins are shown in SEQ ID NO. 1 to 5. Corresponding cDNA sequences are shown in SEQ ID NO. 7 to 11. Exemplary (definitely pathogenic) mutations are: in SNCA: c.88G>C, y.152G>A, c.157G>A, c.158C>A; in LRRK2: p.Asn1437His, p.Arg1441Gly, p.Arg1441Cys, p.Arg1441His, p.Tyr1699Cys, p.Gly2019Ser, p.Ile2020Thr; in VPS35: c.1858G>A, in PRKN: c.101delA; c.101_102delAG, c.53delA, c125G>C, c219_220dupGT, c.337_376del, c.633A>T, c.635G>A, c.785C>G, c.823C>T, c.865T>G, c.931C>T, c.1244C>A, c.1289G>A, C.1292G>T, c.1310C>T, c,1321T>C, c.1358G>A, in PINK1: c.502G>C, c.718G>A, c.813C>A, c.926G>A, c.1040T>C, c.1157G>C, c.1162T>C, c.1226G>T, c.1250A>G, C.1311G>A, c.1366C>T. The presence of pathogenic mutations can be detected, in a DNA sample, e.g., from whole blood, muscle biopsy tissue and/or PBMC homogenate, by Sanger sequencing, SNP-Chip, rtPCR, or by other methods known in the art.

According to an embodiment, the subject has nigrostriatal degeneration or denervation at begin of treatment, preferably confirmed by ¹²³I-loflupane dopamine transporter imaging (DAT-scan).

According to an embodiment, the subject has a Hoehn and Yahr score <4, optionally <3, at begin of treatment. The Hoehn & Yahr score is assessed by the Hoehn & Yahr stage in MDS-UPDRS (The MDS-sponsored Revision of the Unified Parkinson's Disease Rating Scale. International Parkinson and Movement Disorder Society (2008), last updated Aug. 13, 2019; https://www.movementdisorders.org/MDS-Filesl/PDFs/Rating-Scales/MDS-UPDRS_English_FINAL_Updated_August2019.pdf). Accordingly, the Hoehn & Yahr stages are 0: asymptomatic, 1: unilateral involvement only, 2: bilateral involvement without impairment of balance, 3: mild to moderate involvement; some postural instability but physically independent; needs assistance to recover from pull test, 4: severe disability; still able to walk or stand unassisted, 5: wheelchair bound or bedridden unless aided. Preferably, the subject has had optimal symptomatic therapy, not requiring adjustments, for at least 1 month before start of treatment.

According to an embodiment, the subject has no dementia or other neurodegenerative disorder at begin of treatment.

According to an embodiment, the subject has not been diagnosed with atypical parkinsonism, in particular PSP, MSA, CBD, or vascular parkinsonism at begin of treatment.

According to an embodiment, the subject has no metabolic, neoplastic, or other physically or mentally debilitating disorder at begin of treatment.

According to an embodiment, the subject has not used high dose vitamin B3 supplementation, such as 500 mg or more of niacin daily, within 30 days before begin of treatment.

Preferably, the subject is not diagnosed with or does not suffer from one or more of the following: dementia or other neurodegenerative disorder at start of treatment; atypical parkinsonism, in particular PSP, MSA, corticobasal degeneration CBD); or vascular parkinsonism; a psychiatric disorder that would interfere with compliance; a severe somatic illness that would make the individual unable to comply; a metabolic, neoplastic, or other physically or mentally debilitating disorder at start of treatment; and/or a genetically confirmed mitochondrial disease. Optionally, the subject has not used vitamin B3 supplementation, e.g., high dose supplementation, within 30 days before start of treatment. According to one preferred embodiment, the PD is idiopathic (IPD). In the context of the present invention, a subject with idiopathic PD may have a clinical diagnosis according to the MDS clinical diagnostic criteria for Parkinson's disease, as detailed in Postuma R B, Berg D, Stern M, et al. MDS clinical diagnostic criteria for Parkinson's disease. Mov Disord 2015; 30(12): 1591-601 (see in particular Table 1 therein; a clinical diagnosis requires: 1. absence of absolute exclusion criteria; 2. at least two supportive criteria, and 3. no red flags). According to another preferred embodiment, the PD is secondary parkinsonism or drug-induced parkinsonism.

According to an embodiment, the method involves oral administration of NR to the subject. i.e., the preferred route of NR administration is oral. Alternatively or additionally, nicotinamide riboside may be administered by other suitable route(s).

According to an embodiment, nicotinamide riboside is administered in combination with a dopaminergic agent and/or MAO-B inhibitor. Preferably, the MAO-B inhibitor comprises or is selegiline. Preferably, the dopaminergic agent comprises or is levodopa in combination with a decarboxylase inhibitor such as carbidopa or benserazide, with or without the addition of a COMT-inhibitor such as entacapone or tolcapone. Alternatively or additionally, the dopaminergic agent may be a dopamine-agonist such as pramipexole, ropinirole, rotigotine, bromocriptine or pergolide. In one preferred aspect of this embodiment, 8-12 mg, preferably 10 mg selegiline is administered to the subject per day; and 150-800-mg, preferably 300-450 mg levodopa, and 37.5-200 mg, preferably 75-112.5 mg carbidopa are administered to the subject per day; more preferably, 10 mg selegiline is administered to the subject once per day; and 100 mg levodopa and 25 mg carbidopa are each administered to the subject three times per day. In another preferred aspect of this embodiment, 8-12 mg, preferably 10 mg selegiline is administered to the subject per day; and 150-800-mg, preferably 300-450 mg levodopa, and 37.5-200 mg, preferably 75-112.5 mg benserazide are administered to the subject per day; more preferably, 10 mg selegiline is administered to the subject once per day; and 100 mg levodopa and 25 mg benserazide are each administered to the subject three times per day.

In a preferred embodiment, the time since the last administration of a dopaminergic agent (more preferably: levodopa), relative to each respective administration of NR, is −49±72 min, +28±106 min, −26±63 min or −3±99 min; more preferably −26±63 min.

Therapeutic effects associated with the dopaminergic agent include motor symptom improvement (symptomatic). Therapeutic effects associated with the MAO-B inhibitor include mild motor symptomatic effect+a very mild neuroprotective effect; MAO-B inhibitors have been shown of being are able to minimally delay disease progression. Therapeutic effects associated with the combination of the dopaminergic agent with the MAO-B inhibitor include motor symptom control+very mild effect on disease progression.

Without wishing to be bound by theory, it is believed that NR exerts a therapeutic effect in humans both alone as well as in combination with a dopaminergic agent and/or a MAO-B inhibitor.

NR alone is believed to achieve neuroprotection and delay disease progression. However, it is unethical to do trials of novel agents in PD without also giving dopaminergic agent+MAO-B to the patients, so these agents have to be additionally given in clinical studies. NR may be combined with dopaminergic therapy to also provide adequate motor symptom control to patients, i.e.: NR delays/ameliorates the disease; dopaminergic therapy controls existing symptoms. That is, while NR is believed delay/arrest disease progression and even improve symptoms, dopaminergic agents would still add benefit by providing motor symptom control. On the other hand, if patients start in the presymptomatic or premotor phase, and NR arrests further progression, then dopaminergic treatment may not be necessary.

NR may be combined with a MAO-B inhibitor to offer the patient the combined benefits, i.e.: potentially even greater delay of disease progression.

Again, without wishing to be bound by theory, it is believed that the combination of NR+dopaminergic agent+MAO-B is particularly advantageous, from the viewpoints of: (a) providing neuroprotection and substantial effect on disease progression by NR, (b) augmented this effect by the mild but certain effect of the MAO-B, and (c) controlling the motor symptoms by the dopaminergic agent.

According to an embodiment, the overall treatment duration is at least 2, at least 3, at least 6 or at least 12 months, and optionally up to 18, up to 24 or up to 36 months. The maximum duration of the treatment is not particularly limited, and can be for the entire remaining life of the patient.

According to an embodiment, NR is administered as a monotherapy, or as a monotherapy in combination with a dopaminergic agent plus MAO-B inhibitor, and/or one or more methyl donor(s).

According to an embodiment, NR is administered as a pharmaceutically acceptable salt, solvate and/or hydrate thereof. Preferably, the salt is selected from fluoride, chloride, bromide, iodide, formate, acetate, ascorbate, aspartate, benzoate, butyrate, carbonate, citrate, carbamate, formate, gluconate, glutamate, lactate, malate, methyl bromide, methyl sulfate, nitrate, phosphate, propionate, diphosphate, succinate, sulfate, sulfonate, hydrogen tartrate, hydrogen malate, trifluoroacetate, tribromomethanesulfonate, trichloromethanesulfonate, and trifluoromethanesulfonate. Preferably, the salt is a halogenide. More preferably, the salt is a chloride. Alternatively, the salt can be an acidic NR⁺ salt of tartaric or malic acid, such as NR D-hydrogen tartrate, NR L-hydrogen tartrate, NR L-hydrogen tartrate, or NR D-hydrogen tartrate.

Most preferably, the nicotinamide riboside is nicotinamide riboside chloride.

### Pharmaceutical Compositions

The present invention relates, in a further aspect, to a pharmaceutical composition, the composition comprising nicotinamide riboside as defined in any one of the above embodiments. The composition can be for use in a method for treatment according to any of the preceding embodiments or combinations thereof.

In the context of the present invention, the term “pharmaceutical composition” is intended to encompass a product comprising the claimed compound in therapeutically effective amounts, as well as any product that results, directly or indirectly, from combinations of the claimed compounds. Nicotinamide riboside may be incorporated with or without an excipient and used in the form of capsule, tablet, powder, granule, sachet, troche, pill, wafer, gelcap, elixir, suspension, syrup, drops, spray, inhaler, suppository, solution, injection solution, cream, ointment, lotion, gel, patch, depot, or the like.

Preferably, the pharmaceutical composition further comprises a pharmaceutically acceptable excipient. In the context of the present invention, the term “excipient” refers to a carrier, a binder, a disintegrator and/or a further suitable additive for galenic formulations, for instance, for liquid oral preparations, such as suspensions, elixirs and solutions; and/or for solid oral preparations, such as, for example, powders, capsules, gelcaps and tablets. Carriers, which can be added to the mixture, include necessary and inert pharmaceutical excipients, including, but not limited to, suitable suspending agents, lubricants, flavorings, sweeteners, preservatives, coatings, granulating agents, dyes, and coloring agents.

Preferably, the excipient(s) include one or more, more preferably each of microcrystalline cellulose, hydroxypropyl methylcellulose and magnesium stearate.

Preferably, the pharmaceutical composition comprises, more preferably consists of, nicotinamide riboside chloride, microcrystalline cellulose, hydroxypropyl methylcellulose and magnesium stearate.

Preferably, the pharmaceutical composition comprises nicotinamide riboside in an amount of about 0.001% to 100% by weight, more preferably about 0.01% to about 50% by weight, more preferably 0.1% to about 10% by weight.

According to an embodiment, the composition comprises NR as the sole active ingredient, or as the sole active ingredient in combination with one or more of a dopaminergic agent and MAO-B inhibitor, and/or one or more methyl donor(s).

According to particularly preferred embodiments, the pharmaceutical composition comprises or consists of: a) nicotinamide riboside chloride, microcrystalline cellulose, hydroxypropyl methylcellulose and magnesium stearate; or b) nicotinamide riboside chloride, hypromellose, leucine, microcrystalline cellulose, and silicon dioxide; or c) nicotinamide riboside hydrogen malate, microcrystalline cellulose, hydroxypropyl methylcellulose, calcium laurate, silicium dioxide, and one or more methyl donors, preferably betaine (trimethylglycine).

Preferably, in the context of any aspect of the present invention, NR is not administered together with, and the pharmaceutical composition and the dosage form do not comprise one or more of, more preferably any of: epigallocatechin gallate (EGCG); ginsenoside Rg3 or a pharmaceutically acceptable salt thereof; Acetyl L-Carnitine HCL; R-Alpha Lipoic Acid; Rhodiola rosea; ancient peat and apple extract; denosinetriphosphate disodium; pterostilbene; a urolithin; pterostilbene; N-acetylcysteine, L-carnitine tartrate, and serine; a PARP inhibitor; and a mitochondrial uncoupler.

### Dosage Forms

The present invention relates, in a further aspect, to a dosage form for use in a method as defined hereinabove, the dosage form comprising NR or the above-described pharmaceutical composition.

Preferably, the dosage is an oral dosage form; more preferably a capsule. In a particularly preferred embodiment, the dosage form is an oral capsule comprising, preferably consisting of: a) nicotinamide riboside chloride, microcrystalline cellulose, hydroxypropyl methylcellulose and magnesium stearate; or b) nicotinamide riboside chloride, hypromellose, leucine, microcrystalline cellulose, and silicon dioxide; or c) nicotinamide riboside hydrogen malate, microcrystalline cellulose, hydroxypropyl methylcellulose, calcium laurate, silicon dioxide, and one or more methyl donors, preferably betaine (trimethylglycine).

According to an embodiment, the dosage form is preferably for use according to any of the methods described hereinabove, and comprises 250-5000 mg, preferably 500-4000 mg, more preferably 1000-3000 mg, 500-4000 mg or 3000-4000 mg, most preferably 250 mg, 500 mg, 1000 mg, 1500 mg, 2000 mg or 3000 mg NR per dosage unit.

According to another embodiment, a dosage form comprising >2000 mg NR, and optionally one or more pharmaceutically acceptable excipient(s) is provided. Such dosage form may comprise ≥2500 mg, preferably ≥3000 mg, more preferably ≥4000 mg NR per dosage unit. Such dosage form may comprise >2000 mg and ≤5000 mg, preferably 2500-5000 mg, more preferably 3000-4000 mg or 4000-5000 mg, most preferably 3000 mg NR per unit.

In the dosage form(s), NR can be present as a pharmaceutically acceptable salt, solvate and/or hydrate thereof. The salt is preferably one or more selected from fluoride, chloride, bromide, iodide, formate, acetate, ascorbate, aspartate, benzoate, butyrate, carbonate, citrate, carbamate, formate, gluconate, glutamate, lactate, malate, methyl bromide, methyl sulfate, nitrate, phosphate, propionate, diphosphate, succinate, sulfate, sulfonate, hydrogen tartrate, hydrogen malate, trifluoroacetate, tribromomethanesulfonate, trichloromethanesulfonate, and trifluoromethanesulfonate.

Preferably, NR is present as nicotinamide riboside chloride or nicotinamide hydrogen malate, most preferably nicotinamide riboside chloride.

The dosage form may further comprise one or more of: microcrystalline cellulose, hydroxypropyl methylcellulose, hypromellose, calcium laurate, magnesium stearate, silicon dioxide, and one or more methyl donors, preferably betaine (trimethylglycine).

The dosage form is preferably an oral dosage form; most preferably a capsule. Alternatively dosage forms include capsule, tablet, powder, granule, sachet, troche, pill, wafer, gelcap, elixir, suspension, syrup, drops, spray, inhaler, suppository, solution, injection solution, cream, ointment, lotion, gel, patch, depot, orthe like.

In a preferred embodiment, the dosage form is an oral capsule comprising, preferably consisting of: a) nicotinamide riboside chloride, microcrystalline cellulose, hydroxypropyl methylcellulose and magnesium stearate; or b) nicotinamide riboside chloride, hypromellose, leucine, microcrystalline cellulose, and silicon dioxide; or c) nicotinamide riboside hydrogen malate, microcrystalline cellulose, hydroxypropyl methylcellulose, calcium laurate, silicium dioxide, and one or more methyl donors, preferably betaine (trimethylglycine).

### Definitions of Terms and Abbreviations

In the context of the present invention, the term “treatment” is intended to encompass any kind of therapeutic treatment of the human body. The terms “therapy” and “therapeutic treatment” cover prophylactic methods of treating a disease, curative methods of treating a disease, and/or methods for alleviation of symptoms of a disease. The terms “prophylactic treatment”, “preventive treatment” and “preventing” have the same meaning and denote a treatment aiming at maintaining health by preventing ill effects that would otherwise arise. All salt forms of nicotinamide riboside are intended to be embraced by the scope of the present invention.

In the context of the present invention, the term “pharmaceutically acceptable” refers to a compound, ingredient or ion acceptable for use in medicine and health care. Salts, hydrates and solvates which are suitable for use in medicine are those wherein the counter-ion or associated solvent is pharmaceutically acceptable. However, salts, hydrates and solvates having non-pharmaceutically acceptable counter-ions or associated solvents are within the scope of the present invention, for example, for use as intermediates in the preparation of other compounds and their pharmaceutically acceptable salts, hydrates and solvates. Suitable salts according to the invention include those formed with either organic and inorganic acids or bases.

In the context of the present invention, “nicotinamide riboside” or “nicotinamide riboside active ingredient” generally refers to its cationic form, i.e. C₁₁H₁₅N₂O₅⁺, present in salt form with any suitable counterion. Preferably, or unless specified otherwise, weights are expressed as nicotinamide riboside chloride, i.e. C₁₁H₁₅N₂O₅Cl (M=290.7 g/mol). If a counterion X⁻ other than chloride is present, the respective NR weight is to be recalculated based on the ratio of molar weights of C₁₁H₁₅N₂O₅X and C₁₁H₁₅N₂O₅Cl.

| Abbreviation or | |
|---|---|
| special term | Explanation |
| 31P-MRS | Phospohorus-31 Magnetic Resonance Spectroscopy |
| AE | Adverse event |
| CBD | Corticobasal degeneration |
| CRF | Case report form (electronic/paper) |
| CSA | Clinical study agreement |
| CSI | Chemical shift imaging |
| CTC | Common toxicity criteria |
| CTCAE | Common terminology criteria for adverse event |
| DAE | Discontinuation due to adverse event |
| DaTscan | Dopamine transporter scan |
| EC | Ethics Committee, synonymous to Institutional |
| | Review Board (IRB) and Independent |
| | Ethics Committee (IEC) |
| FDG | 18F Fluorodeoxyglucose |
| GCP | Good clinical practice |
| IB | Investigator's brochure |
| ICF | Informed consent form |
| ICH | International Conference on Harmonization |
| IND | Investigational new drug |
| IP | Investigational product (includes active comparator |
| | and placebo) |
| MDS-UPDRS | Movement Disorders Unified Parkinson's Disease |
| | Rating Scale |
| MoCA | Montreal Cognitive Assessment |
| MRAC | Magnetic resonance based attenuation correction |
| MRC | Mitochondrial respiratory chain |
| mtDNA | mitochondrial DNA |
| NAD | Nicotinamide adenine dinucleotide |
| NAD (NAD⁺, | Nicotinamide adenine dinucleotide (oxidized, |
| NADH) | reduced form) |
| NMSQ | Non-Motor Symptoms Questionnaire |
| NMSS | Non-Motor Symptom assessment scale for |
| | Parkinson's Disease |
| NOE | Nuclear Overhauser effect |
| NR | Nicotinamide riboside |
| NRRP | NR-related metabolic pattern |
| OBD | Optimal biological dose |
| PD | Parkinson's Disease |
| PDRP | PD-related spatial covariance pattern |
| PET | Positron emission tomography |
| SAE | Serious adverse event |
| SD | Stable disease |
| SIRT | Sirtuin |
| SNc | Substantia nigra compacta |
| SOP | Standard operating procedure |

### EXAMPLES

### Example 1: Dose Optimization Trial of Nicotinamide Riboside in Parkinson'S Disease

### 1. Protocol Synopsis

### Phase and Study Type

Phase II, Dose-Optimization study
   Investigational Product (IP) (including Nicotinamide Riboside active comparator and placebo):
Placebo
Treatment Duration: 12 weeks

### Objectives:

### Primary Objective:

To determine the Optimal Biological Dose (OBD) for NR, defined as the dose required to achieve: maximal cerebral NAD increase (measured by 31P-MRS or CSF metabolomics), or maximal expression increase in the NRRP (measured by FDG-PET), or maximal proportion of MRS-responders, in the absence of unacceptable toxicity.

### Key Secondary Objectives:

Determine the safety and tolerability of increasing NR doses in PD, measured by the frequency and severity of adverse events, and changes in vital signs and clinical laboratory values.
Determine whether NR-therapy improves clinical dysfunction in PD, and whether this effect is dose-dependent.
Determine the effect of NR therapy on the NAD metabolome and other metabolites in peripheral blood cells and CSF, and whether this effect is dose-dependent.
Determine whether NR-therapy ameliorates proteostasis, via enhancing lysosomal and proteasomal function, and whether this effect is dose-dependent.
Determine whether NR-therapy influences histone acetylation status in PD, and whether this effect is dose-dependent.
Determine whether NR-therapy decreases neuroinflammation and whether this effect is dose-dependent.
Determine whether NR-therapy, in any of the tested doses, affects methylation metabolism. Specifically, whether NR-therapy, in any of the tested doses, leads to decreased availability of methylation substrates and, as a result, any of the following:
   Decreased availability of methyl-donors (e.g., SAM).
   Decreased DNA methylation (globally or at specific sites).
   Decreased synthesis of neurotransmitters like dopamine and serotonin.
   Aberrant folate and one-carbon metabolism

### Experimental Objectives:

Determine the effects of increasing NR-dose on gene and protein expression in PD.
Determine whether NR-therapy influences the gut microbiome in PD, and whether this effect is dose-dependent.

### Outcomes: Primary Outcome

Pairwise between-dose difference in the dose-escalation group (i.e., baseline vs NR 1000 mg, NR 1000 mg vs NR 1500 mg, NR 1500 mg vs NR 2000 mg) in:
   Cerebral NAD levels (measured by 31P-MRS)
   CSF NAD and related metabolite levels (measured by HPLC-MS metabolomics, or the NADmed method)
   NRRP expression (measured by FDG-PET)
Between visit difference in the placebo group (i.e., V1 vs V2, V2 vs V3, V3 vs V4) assessed to determine the specificity of the findings to the NR-therapy.
Between visit difference in the 1000 mg NR group (i.e., V1 vs V2, V2 vs V3, V3 vs V4) assessed to identify any time effects and differentiate those from dose-effects.

### Key Secondary Outcomes:

Pairwise between-dose difference in the dose-escalation group (baseline vs NR 1000 mg, NR 1000 mg vs NR 1500 mg, NR 1500 mg vs NR 2000 mg) in:
   Frequency and severity of adverse events, and changes in vital signs and clinical laboratory values.
   Disease severity, measured by total MDS-UPDDRS and individual subsections (part I-IV score) of MDS-UPDRS
   Levels of metabolites in PBMC and CSF, measured by HPLC-MS and the NADmed method.
   Gene and protein expression levels of factors involved in lysosomal and proteasomal function.
   Levels of histone panacetylation, and levels and genomic distribution of H3K27 and H4K16 acetylation in PBMC, measured by immunoblotting and chromatin immunoprecipitation sequencing (ChIPseq).
   Levels of inflammatory cytokines in serum and CSF, measured using ELISA
   Levels of one carbon metabolism metabolites, measured by HPLC-MS metabolomics in PBMC and CSF; levels of monoamine neurotransmitters in CSF; levels and genomic distribution of DNA methylation, measured by Illumina Infinium MethylationEPIC Kit.
Between visit difference in the placebo group (i.e., V1 vs V2, V2 vs V3, V3 vs V4) is assessed to determine the specificity of the findings to the NR-therapy.
Between visit difference in the 1000 mg NR group (i.e., V1 vs V2, V2 vs V3, V3 vs V4) is assessed to identify any time effects and differentiate those from dose-effects.

### Experimental Outcomes:

Pairwise between-dose difference in the dose-escalation group (baseline vs NR 1000 mg, NR 1000 mg vs NR 1500 mg, NR 1500 mg vs NR 2000 mg) in:
   Gene and protein expression levels in PBMC, measured by RNA sequencing (RNAseq) and proteomics (LC-MS), respectively.
   Gut microbiome, assessed by metagenomics in fecal samples.
Between visit difference in the placebo group (i.e., V1 vs V2, V2 vs V3, V3 vs V4) is assessed to determine the specificity of the findings to the NR-therapy.
Between visit difference in the 1000 mg NR group (i.e., V1 vs V2, V2 vs V3, V3 vs V4) is assessed to identify any time effects and differentiate those from dose-effects.

### Study Design:

Multi-center, double-blinded, randomized, placebo controlled, dose-optimization

### Main Inclusion Criteria:

Clinical diagnosis of idiopathic PD according to the MDS criteria.
123I-loflupane dopamine transporter imaging (DAT-scan) confirming nigrostriatal degeneration.
Hoehn and Yahr score <4 at enrollment.
Age ≥40 years at the time of enrollment.

### Main Exclusion Criteria:

Dementia or other neurodegenerative disorder at baseline visit.
Diagnosed with atypical parkinsonism (PSP, MSA, CBD) or vascular parkinsonism.
Any psychiatric disorder that would interfere with compliance in the study.
Metabolic, neoplastic, or other physically or mentally debilitating disorder at baseline visit.
Use of high dose vitamin B3 supplementation within 30 days of enrollment

### Sample Size:

80 patients (20 in placebo group, 60 in treatment groups)

### Efficacy Assessments:

Primary Outcome: Cerebral NAD levels, CSF NAD and related metabolite levels, NRRP (see details under Outcomes).

### Safety Assessments:

Biochemistry: Routine blood analysis.
Vital signs: pulse, blood-pressure.
Registration of adverse events.

### 2. Study Objectives and Related Endpoints

### 2.1 Primary Objective

The primary objective of the study is to determine the Optimal Biological Dose (OBD) for NR, defined as the dose required to achieve: maximal cerebral NAD increase (measured by 31P-MRS or CSF metabolomics), or maximal expression increase in the NRRP (measured by FDG-PET), or maximal proportion of MRS-responders, in the absence of unacceptable toxicity.

### 2.2 Secondary Objectives & Experimental Objectives

Characterize the safety and tolerability of increasing NR doses in PD, measured by the frequency and severity of adverse events, and changes in vital signs and clinical laboratory values.
Characterize clinical dysfunction improvement in PD by NR-therapy, and dose-dependency of this effect.
Characterize the effect of NR therapy on the NAD metabolome and other metabolites in peripheral blood cells and CSF, and dose-dependency of this effect.
Characterize amelioration of proteostasis by NR-therapy, via enhancing lysosomal and proteasomal function, and dose-dependency of this effect.
Characterize influence of histone acetylation status in PD by NR-therapy, and dose-dependency of this effect.
Characterize decrease in neuroinflammation by NR-therapy, and whether this effect is dose-dependency of this effect.
Characterize to which extent NR-therapy, in any of the tested doses, affects methylation metabolism. Specifically, characterize to which extent NR-therapy, in any of the tested doses, leads to decreased availability of methylation substrates and, as a result, any of the following:
   Decreased availability of methyl-donors (e.g., SAM).
   Decreased DNA methylation (globally or at specific sites).
   Decreased synthesis of neurotransmitters like dopamine and serotonin.
   Aberrant folate and one-carbon metabolism

### Experimental Objectives:

Characterize the effects of increasing NR-dose on gene and protein expression in PD.
Characterize to which extent NR-therapy influences the gut microbiome in PD, and whether this effect is dose-dependent.

### 2.3 Primary Outcomes

The pairwise between-dose difference in the dose-escalation group (i.e., baseline vs NR 1000 mg, NR 1000 mg vs NR 1500 mg, NR 1500 mg vs NR 2000 mg) in:
Cerebral NAD levels (measured by 31P-MRS)
CSF NAD and related metabolite levels (measured by HPLC-MS metabolomics, or the NADmed method)
NRRP expression (measured by FDG-PET)

The between visit difference in the placebo group (i.e., V1 vs V2, V2 vs V3, V3 vs V4) is assessed to determine the specificity of the findings to the NR-therapy. The between visit difference in the 1000 mg NR group (i.e., V1 vs V2, V2 vs V3, V3 vs V4) is assessed to identify any time effects and differentiate those from dose-effects.

### 2.4 Secondary & Experimental Outcomes

The pairwise between-dose difference in the dose-escalation group (baseline vs NR 1000 mg, NR 1000 mg vs NR 1500 mg, NR 1500 mg vs NR 2000 mg) in:
Frequency and severity of adverse events, and changes in vital signs and clinical laboratory values.
Disease severity, measured by total MDS-UPDDRS and individual subsections (part I-IV score) of MDS-UPDRS
Levels of metabolites in PBMC and CSF, measured by HPLC-MS and the NADmed method.
Gene and protein expression levels of factors involved in lysosomal and proteasomal function.
Levels of histone panacetylation, and levels and genomic distribution of H3K27 and H4K16 acetylation in PBMC, measured by immunoblotting and chromatin immunoprecipitation sequencing (ChIPseq).
Levels of inflammatory cytokines in serum and CSF, measured using ELISA
Levels of one carbon metabolism metabolites, measured by HPLC-MS metabolomics in PBMC and CSF; levels of monoamine neurotransmitters in CSF; levels and genomic distribution of DNA methylation, measured by Illumina Infinium MethylationEPIC Kit.

The between visit difference in the placebo group (i.e., V1 vs V2, V2 vs V3, V3 vs V4) is assessed to determine the specificity of the findings to the NR-therapy.

The between visit difference in the 1000 mg NR group (i.e., V1 vs V2, V2 vs V3, V3 vs V4) is assessed to identify any time effects and differentiate those from dose-effects.

### Experimental Outcomes:

The pairwise between-dose difference in the dose-escalation group (baseline vs NR 1000 mg, NR 1000 mg vs NR 1500 mg, NR 1500 mg vs NR 2000 mg) in:
Gene and protein expression levels in PBMC, measured by RNA sequencing (RNAseq) and proteomics (LC-MS), respectively.
   Gut microbiome, assessed by metagenomics in fecal samples.

### 3. Overall Study Design

This is a single-center, phase II, double blinded, randomized, placebo controlled dose-optimization study. Treatment Duration: 12 weeks.

### 4. Study Population

### Number of Patients

80 patients are included in this study: 60 receiving oral NR and 20 receiving placebo.

### Inclusion Criteria

The following condition applies to the prospective patient at screening prior to receiving study agent:
Clinical diagnosis of idiopathic PD according to the MDS criteria ((Postuma R B, Berg D, Stern M, et al. MDS clinical diagnostic criteria for Parkinson's disease. Mov Disord 2015; 30(12):1591-601; Postuma R B, Berg D. The New Diagnostic Criteria for Parkinson's Disease. Int Rev Neurobiol 2017; 132:55-78).
¹²³I-loflupane dopamine transporter imaging (DAT-scan) confirming nigrostriatal degeneration.
Hoehn and Yahr score <4 at enrolment.
Age ≥40 years at the time of enrollment.

### Exclusion Criteria

Patients are excluded from the study if they meet any of the following criteria:
Dementia or other neurodegenerative disorder at baseline visit.
Diagnosed with atypical parkinsonism (PSP, MSA, CBD) or vascular parkinsonism.
Any psychiatric disorder that would interfere with compliance in the study.
Metabolic, neoplastic, or other physically or mentally debilitating disorder at baseline visit.
Use of high dose vitamin B3 supplementation within 30 days before enrollment.

### 5. Treatment

Nicotinamide Riboside (NR) is defined as the Investigational Product(s) (IP). IP includes also active comparator and placebo. NR is provided as Niagen® from Chromadex. Active study drug capsules contain 500 mg NR (as chloride). Placebo contains microcrystalline cellulose, which is be identical in appearance and taste.

### 5.1 Drug Identity, Supply and Storage

NR (Niagen®, Chromadex) and placebo are prepared as identical capsules. The NR and placebo have a 1-year expiry date. Both the NR and placebo are stored in room temperature with temperature <25° C.

### 5.2 Dosage and Drug Administration

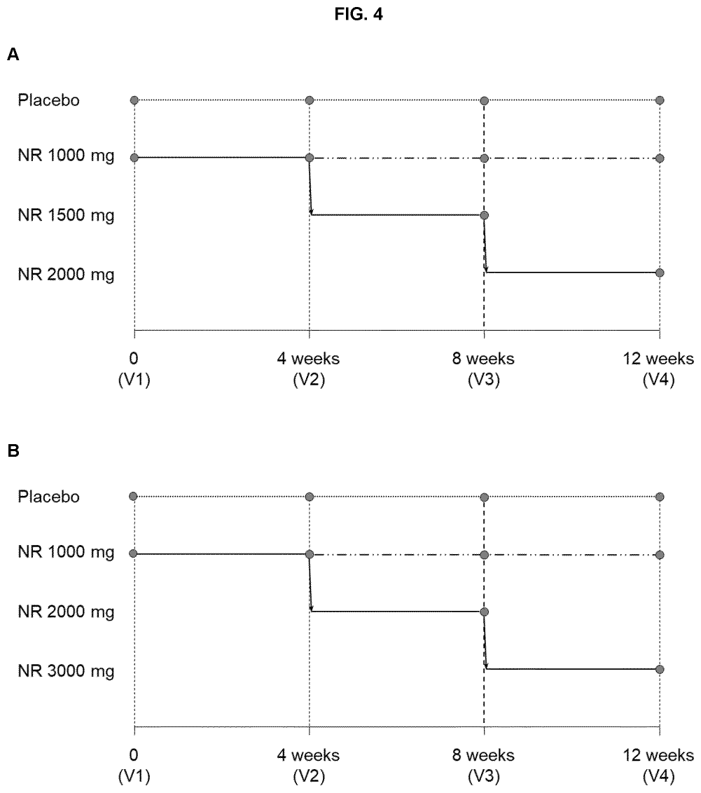

Each NR capsule contains 250 mg or 500 mg of NR. To keep the study fully blinded, all participants receive the same number of daily capsules irrespective of which treatment group they belong to. To achieve this, NR capsules are combined with placebo capsules as necessary (see below, and FIG. 4A):
Patients in the NR 1000 mg group administer orally [1 NR capsule (500 mg)+2 placebo capsules]×2 times daily (1000 mg NR daily in total).
Patients in the NR dose escalation group administer orally the following doses:
   Weeks 0-4: [1 NR capsule (500 mg)+2 placebo capsules]×2 times daily (1000 mg NR daily in total).
   Weeks 5-8: [1 NR capsule (500 mg)+1 NR capsule (250 mg)+1 placebo capsule]×2 times daily (1500 mg daily total).
   Weeks 9-12: [2 NR capsules (500 mg)+1 placebo capsule]×2 times daily (2000 mg daily total).
The placebo group administers orally [3 placebo capsules]×2 times daily.

The study medication is to be taken every day during the treatment period, including prior to study visits.

### 5.3 Duration of Therapy

Therapy duration for the study is 3 months (12 weeks).

### 5.4 Dopaminergic Therapy During Screening and IP Treatment Period

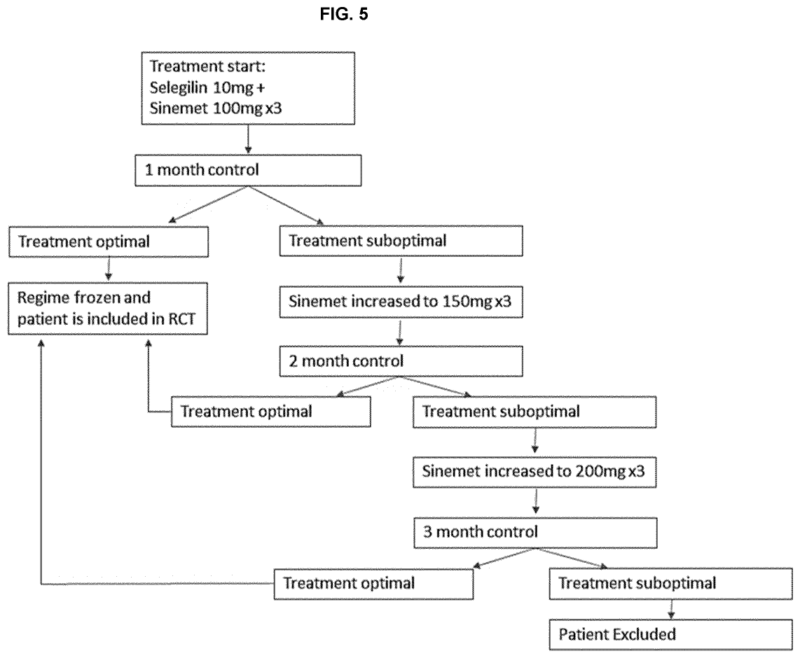

Eligible and consenting men and women with PD are given dopaminergic therapy plus MAO-B inhibitor titrated to optimal clinical effect. The treatment regime is then frozen and remains unchanged for the study period (3 months). Newly diagnosed and/or treatment naïve patients are given Selegiline 10 mg/day PO and Sinemet (levodopa 100 mg+carbidopa 25 mg) or Madopar (levodopa 100 mg+benserazide 25 mg)×3 a day at the first screening visit. Treatment efficacy is assessed upon reexamination by physical or telephone consultation every month. If adequate symptomatic relief is not achieved, the dopaminergic therapy may be increased to 150 mg×3 levodopa until optimal effect or a maximum dose of Sinemet/Madopar 200 mg×3. If adequate symptomatic relief is not achieved on this dose, the patient are excluded. Once optimal effect is reached (i.e., stable treatment for at least 2 weeks, the regime is frozen for the duration of the study period (3 months), see FIG. 5. If adverse effects occur due to the dopaminergic therapy after enrollment, the treatment is adjusted according to good clinical practice. All patients are enrolled to the main study within 3 months after the last screening if inclusion/exclusion criteria are fulfilled. At the end of study visit (month 3), the physician determines (yes/no) whether the patient is still adequately treated for his/her parkinsonism with their current dopaminergic treatment.

### 5.5 Concomitant Medication

There are no restrictions on any other use of medications. All patients should use medications prescribed prior to enrollment in the study. There are no restrictions with respect to starting new medications that are necessary for the patient. The Patient should not take any vitamin B3 supplements for the duration of the study.

### 6. Study Procedures

### 6.1 Flow Chart

TABLE 2
Trial flow chart

| | | | Treatment Period | | | End of |
|---|---|---|---|---|---|---|
| | Screening Period | | Study Visit: | | | study visit |
| | Time | | | | | |
| | First | Next/Last | Visit-1 | | | |
| | Screening | screening¹ | Baseline¹ | Visit-2 | Visit-3 | Visit-4 |
| Time | | | Week | Week | Week | Week |
| | | | 1 | 4 | 8 | 12 |
| Informed consent | X | | | | | |
| MDS Clinical diagnosis | X | | | | | |
| Criteria (MDS CDC) | | | | | | |
| Inclusion/exclusion | X | | | | | |
| Evaluation | | | | | | |
| Informed consent biobank | X | | | | | |
| Anamnestic information⁵ | X | | X | | | |
| Physical Examination³ | X | | X | X | X | X |
| Record of concomitant | X | | X | X | X | X |
| medication | | | | | | |
| Medical history | X | | X | | | |
| Hoehn and Yahr score | X | | X | X | X | X |
| DatScan² | X | | | | | |
| 31P-MRS and FDG-PET | | | X | X | X | X |
| imaging | | | | | | |
| Vital signs^{6,8} | | | X⁸ | X⁶ | X⁶ | X⁶ |
| MDS-UPDRS | | | X | X | X | X |
| NMSS, NMSQ, MOCA and | | | X | X | X | X |
| EQ-5L | | | | | | |
| Blood collection (Whole | | | X¹⁰ | X | X | X |
| blood⁷, PBMC) | | | | | | |
| Cerebrospinal fluid collection | | | X | | | X |
| Fecal sample collection | | | X | | | X |
| Urine sample collection | | | X | | | X |
| Treatment (IP) | | | X⁹ | X⁹ | X⁹ | |
| administration/dispensation | | | | | | |
| Dopaminergic treatment | X | X | | | | X |
| stable⁴ | | | | | | |
| Adverse event | | | X | X | X | X |

¹The patient has to be on a stable dopaminergic treatment. When the patient is on a stable dopaminergic treatment then screening is over and patient can be included to the study. The Dopaminergic treatments and its flow chart is listed in section “Dopaminergic Therapy During Screening and IP treatment period”. There should not be more than 3 months from last screening to baseline visit. The next/last screening is performed as a telephonic consultation.
²DatScan should be performed within 6 weeks prior to the Baseline Study Visit.
³General Neurological examination
⁴See section “Dopaminergic Therapy During Screening and IP treatment period”
⁵Anamnestic information includes: Family history of Neurological illness, smoking history, anamnestic months since first clinical PD symptoms, Occurrence and duration of REM sleep disorder symptoms, occurrence and duration of loss of smell
⁶Blood Pressure, Pulse, Body Weight
⁷CRP, ALAT, ASAT, GT, Bilirubin, ALP, Creatinin, Urea, RBC, Hb, WBC with differential, Platelets, CK, FT4, TSH, B12, Folic acid, homocysteine, Methylmalonic acid, Sodium, Potassium. Biobanking: see lab manual for details.
⁸Height (measured at Baseline visit)
⁹To ensure correct dosages during dose escalation and if necessary to resupply.
¹⁰Women of childbearing potential also have a pregnancy test performed.

### 6.2 By Visit

### 6.2.1 Screening Visits/Before Start of Investigational Product (IP)

The first screening visit aims to determine if the patient is eligible to be included in the study. A full physical examination and anamnestic medical history is performed. If the patient fulfils the inclusion/exclusion criteria and gives informed consent, dopaminergic treatment is initiated/adjusted as described in the treatment flowchart in section Dopaminergic Therapy During Screening and IP treatment period”

If the patient is optimally treated with dopaminergic treatment and fulfils the inclusion/exclusion criteria, then the patient is deemed ready for enrolment and can be referred to DatScan to confirm nigrostriatal degeneration. If changes are made to the dopaminergic treatment, then the subject is contacted by phone after 2-6 weeks to assess if the treatment is optimal. If the patient is optimally treated, this dopaminergic treatment is frozen for the remainder of the study and the patient is ready for enrollment. The patient is then referred to DatScan and MRI examination. Following these and provided a positive DatScan, the patient is called in for the baseline study visit (week 0 study visit). The Baseline study visit should be within 6 weeks from the time the DatScan was performed. The patient should at screening be advised to stop using any Vit B3 supplement to fulfill inclusion criteria. There should not be more than 3 months from last screening to baseline visit.

### Screening Checklist:

Informed consent
Physical examination (general neurological examination) at first screening
MDS clinical diagnosis Criteria
Record current use of medication. Advise to stop any use of vit B3 supplements
Introduce dopaminergic treatment as described in section, “Dopaminergic Therapy During Screening and IP treatment period”.
If patient is optimally treated on one the predefined dopaminergic regimes in section “Dopaminergic Therapy During Screening and IP treatment period”, the patient can be referred to DatScan with subsequent baseline study visit within 6 weeks of the imaging date.

### 6.2.2 Baseline/Week 0

At the first study visit the investigator needs to verify the informed consent for the study and offer the subject to sign the informed consent for storage and analysis of biological material Verify anamnestic information gathered at screening, current use of medication and medical history. Verify fulfillment of inclusion and exclusion criteria. If the subject is enrolled (fulfills the inclusion/exclusion criteria), the study medication is dispensed at the visit to the subject by the study nurse. The patient is instructed to take the study medication every day for the remainder of the study. The study medication or placebo is to be taken as outlined in section “Dosage and Drug Administration”. The capsules are taken in the morning and evening. There is no specified time of day the dosages should be taken, only that they should be taken with about 12 hours apart if possible. If a dose is missed, the patient can take the missed dose as soon as it is remembered, provided it is shorter time to the missed dose than the next scheduled dose. There are no restrictions with respect to combining the dose with other medication and/or food. Study medication should, if possible, be taken prior to study visits.

### 6.2.3 During Treatment

See flowchart (Table 2) for which clinical examinations are performed at each study visit.

### 6.3 Laboratory Tests

Below is provided an overview of the biological material collected for biobanking. At different time points during the clinical trial, the following biological samples are collected, processed and stored based on standard operating procedures: whole blood, serum, plasma, PBMCs, blood cells. The samples are collected, prepared, and stored as described in detail below.

Table 3. Biological material that collected for hematology, biochemistry, hormone and serology analyses and for serum hCG pregnancy test.

Flow chart

Visits

| | | | |
|---|---|---|---|
| V1/Baseline | V2 | V3 | V4 |

Week

| | 0 | 4 | 8 | 12 |
|---|---|---|---|---|
| Routine: | | | | |
| hCG¹ | X | | | |
| CRP | X | X | X | X |
| ALAT | X | X | X | X |
| ASAT | X | X | X | X |
| GT | X | X | X | X |
| Bilirubin | X | X | X | X |
| ALP | X | X | X | X |
| Creatinin | X | X | X | X |
| Urea | X | X | X | X |
| RBC | X | X | X | X |
| Hb | X | X | X | X |
| WBC with differential | X | X | X | X |
| Platelets | X | X | X | X |
| CK | X | X | X | X |
| FT4 | X | X | X | X |
| TSH | X | X | X | X |
| B12 | X | X | X | X |
| Folic acid | X | X | X | X |
| homocystein | X | X | X | X |
| Methylmalonic acid | X | X | X | X |
| Sodium | X | X | X | X |
| Potassium | X | X | X | X |
| Biobank: | | | | |
| EDTA whole blood | X | | | |
| Snap-frozen whole blood | X | X | X | X |
| PAXgene blood for RNA | X | X | X | X |
| *Fullblood for PBMCs | X | X | X | X |
| Serum | X | X | X | X |
| Fecal sample | X | | | X |
| Urine sample | X | | | X |
| Cerebrospinal fluid | X | | | X |

TABLE 4
Safety laboratory (blood). All safety laboratory
parameters are collected at the timepoints as indicated
in the Flow Chart in Table 3 above, and include
hematology, liver enzymes/parameters, clinical
chemistry, thyroid status. Safety parameters evaluated
during the study are listed below.

| | |
|---|---|
| Hematology | |
| Hemoglobin | Platelet count/thrombocytes |
| WBC/leukocytes | Differentials: neutrophils, |
| | eosinophils, basophils, |
| | monocytes, lymphocytes |
| Liver enzymes/parameters | |
| ALAT (alanine transaminase, SGPT) | Alkaline phosphatase |
| ASAT (aspartate transaminase, SGOT) | GT (glutamyl transferase) |
| | Bilirubin total, fractionated |
| | if increased |
| Clinical chemistry | |
| Creatinine | Potassium |
| CRP | Sodium |
| Thyroid status | |
| TSH (thyroid stimulating hormone) | |
| Free T4 | |

TABLE 5
Sampling in sub-studies (Biobank).

| Type of | |
|---|---|
| sample | Analyses |
| Whole | DNA |
| blood | |
| Serum | Protein patterns |
| | Quantification of neurofilament light chain and |
| | neurological biomarkers with quanterix Simoa |
| | (https://www.quanterix.com/therapeutic-areas/cns-biomarkers) |
| PBMC | Immunophenotyping |
| prepa- | Functional markers |
| ration | Mass/flow cytometry |
| | Gene expression |
| | Sequencing |
| | Sorting |
| | Proteomics |
| | Immunohistochemistry |
| | Somatic mutations |
| | DNA methylation |
| | Histone modifications |
| | DNA modifications |

### 6.4 Imaging Studies

1) DAT-scan, performed according to standard clinical routine, will confirm the presence of nigrostriatal degeneration.
2) Structural MRI will assess total and regional brain volume in anatomical areas affected by PD, structural and functional brain connectivity. Each examination will include: 3D T1- and T2-weighted MRI (sMRI), advanced diffusion imaging allowing modelling of restricted or hindered diffusion (dMRI).
3) fMRI: whole brain resting state BOLD MRI (rs-fMRI). Gradient reversal correction is applied to correct functional image data for susceptibility artifacts.
4) 311P-MRS is conducted on a 3T Biograph mMR MR-PET scanner (Siemens Healthcare, Germany) to assess the intracerebral concentration of NAD, analogous to FIG. 1A-C.
5) FDG-PET imaging is performed on the same MR-PET scanner and in the same session, to assess the metabolic response to NR treatment (analogous to FIG. 1D-E). Following standard preprocessing protocols and spatial normalization, the NRRP is assessed using ordinal trends/canonical variates analysis (OrT/CVA), a supervised form of principal component analysis (PCA) (Bender A, Krishnan K J, Morris C M, et al. High levels of mitochondrial DNA deletions in substantia nigra neurons in aging and Parkinson disease. Nat Genet 2006; 38(5):515-517). This multivariate approach is designed to detect and quantify regional covariance patterns (i.e., metabolic networks) for which expression values (i.e., subject scores) increase or decrease with treatment in all or most of the subjects.

The MRI protocol is summarized below.
Scanner: Siemens Biograph mMR (PET/MR). Software: E11P. Coils: Siemens mMR Head/Neck coil; multinucleus 31P 1H head coil (Rapid).

### Protocol:

Positioning: Localizer
   Autoalign (if possible)
MRI recording 3D T1 (sagital, 1×1×1 mm)
   3D T2 FLAIR (sagital, 1×1×1 mm)
   2D T2 axial (4 mm slice thickness, angle relative to CC/ACPC)
   2D DTI (axial, 2×2×2 mm, angle relative to CC/ACPC)
   (DWI med 3 retninger dersom ikke DTI mulig)
   2D fMRI resting state (2.4×2.4×3 mm, axial slices, angle relative to CC/ACPC)
   31P-MRS opptak: CSI (15 min), multinucleus coil.
Total recording time: 60 min including CSI
Comments: Eyes closed for fMRI recording

### 6.5 Molecular Analyses and Multi-Omics

1) Metabolomics analyses are performed in PBMC, muscle and CSF, using liquid chromatography-mass spectrometry (LC-MS) as described (Trammell S A, Brenner C. Targeted, LCMS-based Metabolomics for Quantitative Measurement of NAD(+) Metabolites. Computational and structural biotechnology journal 2013; 4:e201301012). Absolute metabolite concentrations are determined using in house standards. We will assess the entire NAD-metabolome, and key-metabolites involved in the Krebs' cycle, fatty acid beta-oxidation, and methylation reactions (e.g., SAM, homocysteine, folate).
2) Gene and protein expression. The transcriptome is mapped in PBMC and muscle by RNA-sequencing, using ribosomal depletion and sequencing at 125 bp paired-end and 100 million read pairs per sample, as previously described (Nido G S, Dick F, Toker L, et al. Common gene expression signatures in Parkinson's disease are driven by changes in cell composition. Acta Neuropathol Commun 2020; 8(1):55). Quantitative proteomics is performed in PBMC, muscle and CSF, using TMT (Tandem Mass Tags) labeling and mass spectrometry (LC-MS/MS Q-Exactive HF).
3) Histone acetylation profiling. First, quantitative changes in global histone acetylation status in PBMC and muscle, by immunoblotting with a pan-acetyl-lysin antibody are assessed. Next, acetylation levels of specific lysine residues (e.g., H3K27 and H4K16) are assessed with targeted immunoblotting. Finally, genome-wide changes in the acetylation status of histone lysine residues found to be quantitatively altered by the treatment, are assessed by chromatin-immunoprecipitation sequencing (ChIP-Seq), as described in Toker L, Tran G T, Sundaresan J, et al. Genome-wide histone acetylation analysis reveals altered transcriptional regulation in the Parkinson's disease brain. Molecular Neurodegeneration 2021; 16(1):31.
4) Inflammatory cytokine concentration is determined in CSF using ELISA, e.g., using the Human Cytokine Magnetic 35-plex panel (Invitrogen), detecting FGF-Basic, IL-1 beta, G-CSF, IL-10, IL-13, IL-6, IL-12, RANTES, Eotaxin, IL-17A, MIP-1 alpha, GM-CSF, MIP-1 beta, MCP-1, IL-15, EGF, IL-5, HGF, VEGF, IL-1 alpha, IFN-gamma, IL-17F, IFN-alpha, IL-9, IL-1RA, TNF-alpha, IL-3, IL-2, IL-7, IP-10, IL-2R, IL-22, MIG, IL-4, IL-8. CSF and serum samples were analyzed according to the manufacturer's recommendations and measured on a BioPlex 200 instrument (BioRad).
5) DNA methylation is mapped using the Illumina Infinium Epic Chip.
6) Neurotransmitter levels. Monoamine levels is determined in the CSF using HPLC-MS.
7) Gut microbiome. This is assessed by metagenomic analyses in fecal samples, as described (Romano S, Savva G M, Bedarf J R, et al. Meta-analysis of the Parkinson's disease gut microbiome suggests alterations linked to intestinal inflammation. npj Parkinsons Dis. 2021; 7(1):1-13).

### 7. Safety and Tolerability Assessments

Safety is monitored by the assessments described below as well as the collection of AEs at every visit. For the assessment schedule refer to study flow chart in Table 2.

### 8. Safety Monitoring and Reporting

Events meeting the criteria and definition of an adverse event (AE) or serious adverse event (SAE) are documented. Each patient is instructed to contact the investigator immediately should they manifest any signs or symptoms they perceive as serious. The methods for collection of safety data are described below.

### 8.1 Definitions

### 8.1.1 Adverse Event (AE)

An AE is any untoward medical occurrence in a patient administered a pharmaceutical product and which does not necessarily have a causal relationship with this treatment. An adverse event (AE) can therefore be any unfavorable and unintended sign (including an abnormal laboratory finding), symptom, or disease temporally associated with the use of a investigational product, whether or not related to the investigational product. The term AE is used to include both serious and non-serious AEs. If an abnormal laboratory value/vital sign is associated with clinical signs and symptoms, the sign/symptom should be reported as an AE and the associated laboratory result/vital sign should be considered as additional information that must be collected on the relevant CRF. Only intensity 2 and 3 is registered as AE. An AE has to interfere with everyday life to be of intensity 2 or 3.

### 8.1.2 Serious Adverse Event (SAE)

Any untoward medical occurrence that at any dose:
Results in death
Is immediately life-threatening
Requires in-patient hospitalization or prolongation of existing hospitalization
Results in persistent or significant disability or incapacity
Is a congenital abnormality or birth defect
Is an important medical event that may jeopardize the subject or may require medical intervention to prevent one of the outcomes listed above.

Medical and scientific judgment is to be exercised in deciding on the seriousness of a case. Important medical events may not be immediately life-threatening or result in death or hospitalization, but may jeopardize the subject or may require intervention to prevent one of the listed outcomes in the definitions above. In such situations, or in doubtful cases, the case should be considered as serious. Hospitalization for administrative reason (for observation or social reasons) is allowed at the investigator's discretion and will not qualify as serious unless there is an associated adverse event warranting hospitalization.

### 8.2 Time Period for Reporting AE and SAE

Recording AE and SAEs begins after baseline (week 0) and continue to be monitored and registered throughout the duration of the study up until 7 days after last study visit.

During the course of the study all AEs and SAEs are proactively followed up for each patient; events should be followed up to resolution, unless the event is considered by the investigator to be unlikely to resolve due to the underlying disease. Every effort should be made to obtain a resolution for all events, even if the events continue after discontinuation/study completion.

### 8.3 Recording of Adverse Events

If the patient has experienced adverse event(s), the following information is recorded: the nature of the event(s) is described by the investigator in precise standard medical terminology (i.e. not necessarily the exact words used by the patient); the duration of the event is described in terms of event onset date and event ended data; and the intensity of the adverse event: Only intensity 2 and 3 is registered as AE.

### 8.3.1 Assessment of Intensity

An assessment of intensity for each AE and SAE reported during the study is made to assign it to one of the following categories:
1 Mild: An event that is easily tolerated by the participant, causing minimal discomfort and not interfering with everyday activities.
2 Moderate: An event that causes sufficient discomfort to interfere with normal everyday activities.
3 Severe: An event that prevents normal everyday activities. An AE that is assessed as severe should not be confused with an SAE. Severe is a category utilized for rating the intensity of an event; and both AEs and SAEs can be assessed as severe.

The Causal relationship of the event to the study medication is assessed as one of the following:
Unrelated: There is not a temporal relationship to investigational product administration (too early, or late, or investigational product not taken), or there is a reasonable causal relationship between non-investigational product, concurrent disease, or circumstance and the AE.
Unlikely: There is a temporal relationship to investigational product administration, but there is not a reasonable causal relationship between the investigational product and the AE.
Possible: There is reasonable causal relationship between the investigational product and the AE. Dechallenge information is lacking or unclear.
Probable: There is a reasonable causal relationship between the investigational product and the AE. The event responds to dechallenge. Rechallenge is not required.
Definite: There is a reasonable causal relationship between the investigational product and the AE.

It is important to distinguish between serious and severe AEs. Severity is a measure of intensity whereas seriousness is defined by the criteria in Section “Definitions”. An AE of severe intensity need not necessarily be considered serious. For example, nausea that persists for several hours may be considered severe nausea, but is not an SAE. On the other hand, a stroke that results in only a limited degree of disability may be considered a mild stroke, but would be an SAE.

### 9. Statistical Methods and Data Analysis

### 9.1 Determination of Sample Size

Our primary null hypothesis (H₀) is that the NR-induced increases in cerebral NAD levels (measured by 31P-MRS), CSF NAD-metabolites, or NRRP expression (measured by FDG-PET) are not dose-responsive. The alternative hypothesis (H_{A}) is that at least one of these three measures is dose responsive. In the NADPARK study, all three measures showed a highly significant increase in the group receiving 1000 mg NR compared to the placebo group. Among the three measures, cerebral NAD levels showed the weakest effect size and highest standard deviation (SD). Therefore, we chose to base the power estimation for the N-dose study on this measure, assuming it is the least likely to show a difference. In the NADPARK study, treatment with 1000 mg of NR led to an increase in cerebral NAD-levels by 38% (SD=20%) in the treatment group, whereas the change in the placebo group was negligible at −0.43% (SD=23%). Dose-effects are often non-linear and tend to plateau with increasing dose. Therefore, under the H_{A}, we assume that further increase in cerebral NAD levels is 50% and 60% of placebo values, in the 1500 mg NR and 2000 mg NR groups, respectively. Based on these assumptions, and given a type I error rate of 5% (α=0.05) and a type II error rate of 10% (β=0.1, power=90%), we estimated that a sample size of 17 individuals per group is required. Accounting for drop-out and statistical safety margin, we estimate that the study requires 20 subjects per group.

To confirm that this sample size will provide adequate power, we performed an additional estimation for the NRRP expression. Treatment with 1000 mg of NR gave rise to a significant increase in NRRP expression in the NR treatment group relative to the placebo group (0.68±0.86 vs. −0.33±1.37; mean±SD; p<0.02; Mann-Whitney U test, two-tailed), corresponding to a large effect size (ES) of 0.883. Assuming conservatively that mean NRRP expression will further increase to 0.85 and 1.00 respectively in the 1500 mg NR and 2000 mg NR, we estimated that the proposed sample size of 20 subjects in each group would yield 91% power (α=0.05; ES=0.883) to detect significant changes in NRRP expression between the NR groups and the placebo group.

For the secondary outcomes, we assume that the metabolomic, transcriptomic and inflammatory cytokine analyses will have sufficient power, since they produced very large effect sizes and highly significant results in the NADPARK study with 15 individuals per group. In our previous experience, a sample size of 20 per group should be sufficient to detect treatment-induced differences of biological relevance.

### 9.2 Analyses

All statistical analysis is planned after the completion of the study. All randomized patients are included in the primary analyses. In the case of missing assessments, the subject is excluded. The between visit change in the primary and secondary outcomes are compared within each group by paired Student's t-test (or non-parametric tests according to normality). For PET analyses, changes in network scores with treatment are evaluated for each group separately using permutation tests. Relationships between network values, brain NAD levels and MDS-UPDRS motor ratings or between treatment-related changes in these variables are evaluated using Pearson's product-moment correlations, whereas Spearman rank-order correlation coefficients are computed for non-normally distributed variables. Metabolites and inflammatory cytokines are assessed in a similar way. Omics data undergo rigorous quality control and filtering according to established best practice procedures. The between visit change in this data is assessed by a pairwise comparison between each NR group and the placebo group, using linear models with appropriate covariates as we have previously described. Comparison of adverse events and abnormal laboratory test results between the treatment and placebo groups is done with chi-square tests.

### 9.3 Statistical Analysis

### 9.3.1 Dependent Variable

### Primary Analysis:

Cerebral NAD levels (measured by 31P-MRS)
Level of CSF NAD-metabolites (measured by LS-MS)
Level of NRRP expression (measured by FDG-PET)

### Secondary Analysis:

Adverse effects, categorized as either mild, moderate or severe.
Routine blood tests.
Expression of RNA and protein of genes and pathways involved in proteasomal and lysosomal biogensis and function.
Histone panacetylation levels
Levels of specific lysine residues H3K27 and H4K16 acetylation
Genome-wide distribution of histone lysine residues found to be quantitatively altered by the treatmed (measured by ChIP-Seq).
Level of inflammatory cytokines in patient CSF.
Total MDS-UPDRS and each subsection I-III of MDS-UPDRS. Assessed by the MDS-UPDRS questionnaire.
NMSQ total score. Assessed by the NMSQ questionnaire.
NMSS total score. Assessed by the NMSS questionnaire.
MoCA total score. Assessed by the MoCA questionnaire.
EQ-5L score. Assessed by the EQ-5L questionnaire.
Hoehn & Yahr Stage. Assessed by the Hoehn & yahr stage in MDS-UPDRS.

### 9.3.2 Statistical Hypothesis

Primary analysis: Primary null hypothesis (H₀) is that the NR-induced increases in cerebral NAD levels (measured by 31P-MRS), CSF NAD-metabolites and NRRP expression (measured by FDG-PET) are not dose-responsive. The alternative hypothesis (H_{A}) is that at least one of these three measures is dose responsive.

Secondary analysis: Mean change between and within the NR arm, NR dose escalation arm and placebo arm for the following parameters:
Adverse effects, categorized as either mild, moderate or severe.
Routine blood tests.
Expression of RNA and protein of genes and pathways involved in proteasomal and lysosomal biogensis and function.
Histone panacetylation levels.
Level lysine residues H3K27 and H4K16 acetylation.
Level of genome-wide distribution of histone lysine residues found to be quantitatively altered by the treatmed (measured by ChIP-Seq).
Level of inflammatory cytokines in CSF.
Total MDS-UPDRS and each subsection I-III MDS-UPDRS score.
NMSQ total score.
NMSS total score.
MoCA total score.
EQ-5L total score.
Hoehn & Yahr stage.
Lower number of stage increases (nominal) in Hoehn and Yahr in the NR arm and NR dose escalation arm compared to the placebo arm.

### 10. Conclusions

As a result of the study, if the 2000 mg NR dosage does not have substantially higher efficacy than 1500 mg NR, the highest meaningful dose is determined as 1500 mg for central nervous system (CNS) diseases, in particular PD.

If 2000 mg NR has substantially higher efficacy than 1500 mg NR but causes unacceptable toxicity, first the nature of the toxicity is analysed, and it is determined whether this can be mitigated by: (i) modifying NR and/or (ii) combining NR with compounds counteracting the toxic effects (e.g., methylation donors to counteract methylation depletion). If toxicity is not readily addressable (and until it, potentially, becomes addressed), the maximal safe NR dose is determined as 1500 mg.

If 2000 mg NR has substantially higher efficacy than 1500 mg NR and is safe but not tolerable, the tolerability issue is analysed, and either modify NR and/or combine NR with compounds counteracting the adverse symptoms is attempted. If tolerability is not readily addressable (and until it, potentially, becomes addressed), that the maximal tolerable NR dose is determined as 1500 mg.

If 2000 mg NR has substantially higher efficacy than 1500 mg NR and is both safe and tolerable, it can be maintained. The maximal NR dose can be higher (e.g., 3000-5000 mg).

### Example 2: Further Dose Optimization Trial of Nicotinamide Riboside in Parkinson'S Disease

The study is performed identical manner as that described in Example 1, except for the doses, which are as follows (see FIG. 4B):
Patients in the NR 1000 mg group administer orally.
Patients in the NR dose escalation group administer orally the following doses:
   Weeks 0-4: 1000 mg NR daily in total;
   Weeks 5-8: 2000 mg NR daily in total;
   Weeks 9-12: 3000 mg daily in total.

As a conclusion, if the 3000 mg NR dosage does not have substantially higher efficacy than 2000 mg NR, the highest meaningful dose is determined as 2000 mg for central nervous system (CNS) diseases, in particular PD.

If 3000 mg NR has substantially higher efficacy than 2000 mg NR but causes unacceptable toxicity, first the nature of the toxicity is analyzed, and it is determined whether this can be mitigated by: (i) modifying NR and/or (ii) combining NR with compounds counteracting the toxic effects (e.g., methylation donors to counteract methylation depletion). If toxicity is not readily addressable (and until it, potentially, becomes addressed), the maximal safe NR dose is determined as 2000 mg.

If 3000 mg NR has substantially higher efficacy than 2000 mg NR and is safe but not tolerable, the tolerability issue is analyzed, and either modify NR and/or combine NR with compounds counteracting the adverse symptoms is attempted. If tolerability is not readily addressable (and until it, potentially, becomes addressed), that the maximal tolerable NR dose is determined as 2000 mg.

If 3000 mg NR has substantially higher efficacy than 200 mg NR and is both safe and tolerable, it can be maintained. The maximal NR dose can be higher (e.g., 4000-10000 mg).

### Example 3: NR-Safe: A Randomized, Double-Blind Safety Trial of High Dose Nicotinamide Riboside in Parkinson'S Disease

Nicotinamide adenine dinucleotide (NAD) replenishment therapy with nicotinamide riboside (NR) has shown promise in preclinical and clinical studies on Parkinson's disease (PD) and other neurodegenerative disorders.

To assess the safety of high-dose NR therapy, we performed a double-blind, randomized phase I trial of 3000 mg NR daily in PD. 20 patients were randomized on NR or placebo and followed for a period of 4 weeks. No adverse events or signs of toxicity that were likely attributed to NR were observed. NR-recipients exhibited a significant improvement in total MDS-UPDRS (−10.7±9.94, p=0.007). Furthermore, NR-recipients exhibited a pronounced augmentation of the NAD metabolome with up to 5-fold increase in blood NAD⁺ levels. A mild initial increase in homocysteine was observed in the NR group in serum, but not whole blood samples, while metabolite levels reflecting the integrity of the methyl donor pool remained intact.

Our results establish that oral NR treatment at a dose of 3000 mg daily for a 4 week period is safe, induces a pronounced augmentation of the NAD metabolome, and suggest a clinical symptomatic improvement in PD. Our findings allow for a dose range extension of NR employed in clinical trials (e.g., to 3000 mg daily).

### Introduction

To determine the safety of high-dose NR therapy, we performed a double-blind, randomized phase I trial of 3000 mg NR daily in individuals with PD. The primary outcome of the study was the incidence of treatment-associated moderate and severe adverse events (AEs). Secondary outcomes were the between-group difference in treatment-associated mild AEs, changes of the NAD metabolome in blood and urine, and change in the clinical severity of PD, measured by MDS-UPDRS. Exploratory outcomes were, among others, the between-group difference in the change of serum homocysteine levels and of fasting blood glucose and serum insulin levels.

### Results

### Study Population

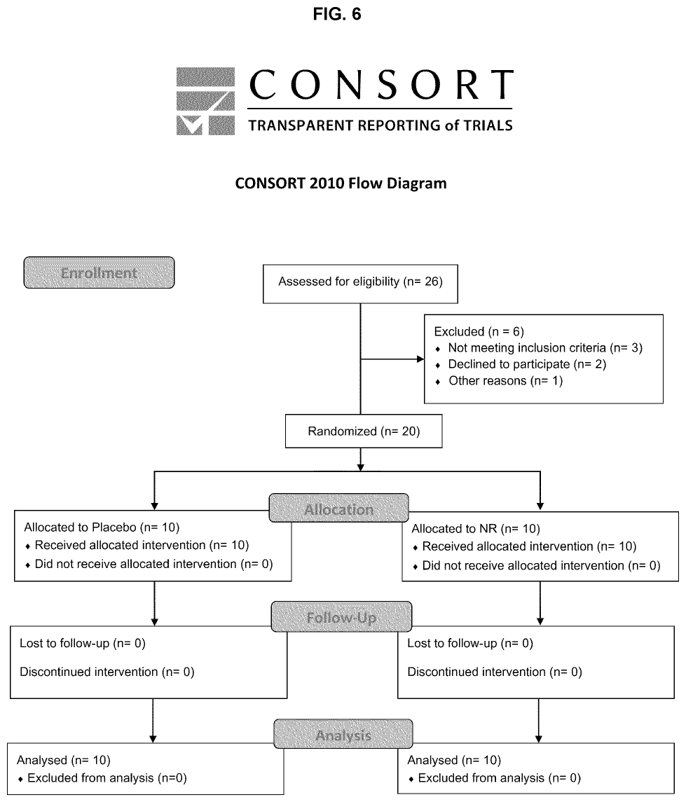

In total, 26 individuals with PD were screened, and 20 eligible participants were enrolled, all of whom completed the study (FIG. 6). There were no significant differences in sex, age, BMI, time since diagnosis of PD, and baseline MDS-UPDRS between the NR and placebo group (Table 6). Drug compliance was similar in both groups (NR: 95.6±2.45%; Placebo: 94.4±3.27%, paired Wilcoxon test: p=0.52). There were no significant differences in the frequency of co-morbid medical conditions.

Samples from all individuals were included in the analyses of laboratory values, including those with abnormal values at baseline. One participant in the placebo group did not fast at any visit due to insulin-treated diabetes mellitus, and one participant in the NR group did not fast at baseline due to omission. These two individuals were omitted from analyses of values depending on fasting samples (i.e., insulin, lipids, and glucose). Two participants did not use levodopa and were therefore excluded in the analysis of the time since levodopa. Participants did not fast on visits other than baseline (visit 1, V1) and last visit (visit 7, V7), because this interfered too much with their daily function. One participant in the NR group was removed from the analysis of urea, sodium, potassium and creatinine due to data entry error. HCG was negative at baseline for all female participants. The groups were similar at baseline with regards to demographics, clinical safety tests, MDS-UPDRS scores and metabolomic parameters.

High Dose NR Treatment was not Associated with Adverse Events.

42 adverse events (AEs) were observed overall, 25 of which in the NR group and 17 in the placebo group. 9/10 participants in the NR group and 8/10 participants in the placebo group experienced at least one AE (Table 7). All AEs were graded as mild, and there was no significant difference in the frequency of adverse events between both groups. The most common adverse events observed in the NR group were classified as extrapyramidal disorder (number of events/number of affected individuals, n=3/3), headache (n=3/3), tremor (n=2/2), muscle cramp (n=2/1), fatigue (n=2/2), nausea (n=2/2) and dyspepsia (n=2/2). All cases of dyspepsia and nausea were resolved at the end of the follow up period. One event each of fatigue, headache and muscle cramps was registered as ongoing at the end of the study. Regarding extrapyramidal disorders, one participant reported increased dyskinesia during the study causing the participant to reduce their daily levodopa dose, while another participant reported increased rigidity after treatment cessation with NR, and the third participant reduced their levodopa dosage during the study due to reduced subjective need of treatment. Regarding tremor, one participant experienced increased tremor during the study, whereas another experienced increased tremor after cessation of the study drug. One case of non-painful maculo-papular rash was seen in the NR group. This participant had recently been treated by their general practitioner for the same rash and, thus, the causal relationship was deemed unlikely. Importantly, no painful flushing was reported, which can occur with nicotinic acid (NA) supplementation as NAD precursor at dosages above 50-100 mg¹. 1 Benyó, Z. et al. GPR109A (PUMA-G/HM74A) mediates nicotinic acid-induced flushing. J Clin Invest 115, 3634-3640 (2005).

No significant changes in systolic or diastolic blood pressure, pulse or weight were seen between visit 1 and visit 7 (i.e., Δ=V7−V1) in either group, nor were there significant between-group differences in the mean change for each variable (Table 8). Electrocardiography (ECG) revealed no changes of clinical significance. Two patients in the NR group developed asymptomatic bradycardia (58 beats/min and 46 beats/min).

High Dose NR Treatment was Associated with Clinical Improvement.

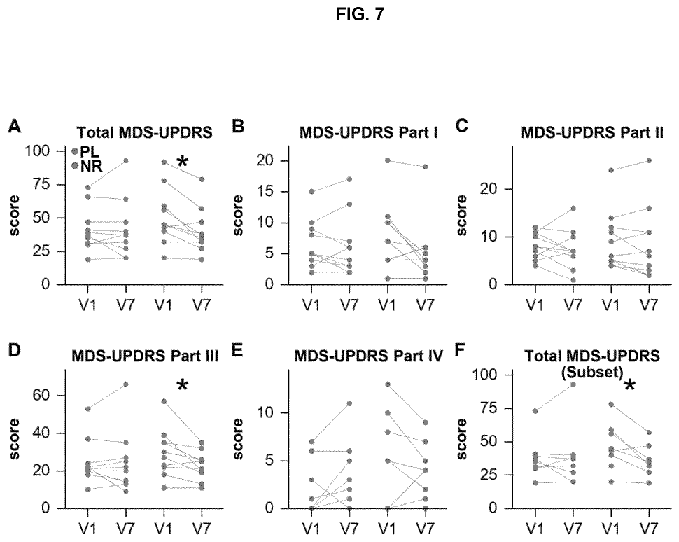

The NR group, but not the placebo group, showed a statistically significant decrease in the total MDS-UPDRS (I-IV) score between V1 and V7 (NR: V1: 51±21.28 vs V7: 40.3±17.1; mean change −10.7±9.94; paired t-test, p=0.007; Placebo: V1: 41.8±16.46 vs V7: 41.8±22.23; mean change 0±9.59; paired t-test, p=1; Table 9 and FIG. 7A-E). This change in the NR group was primarily driven by MDS-UPDRS part III (V1: 29.7±12.85 vs V7: 22.7±7.55; paired t-test, p=0.021), and to a minor extent by part I (NR: −2.20±3.52, t-test, p=0.079). Interestingly, we noted that the “mean time since the last levodopa dose” was shorter on day 28 compared to baseline in the NR group (−49±71.93 min) and longer in the placebo group (+28.33±105.59 min). While all participants were assessed at ON-state and reported no wearing-off, one cannot exclude the possibility that the observed change in UPDRS was, at least in part, driven by the difference in levodopa dosing intervals. To investigate this further, we also compared a subgroup of 8 participants per group with similar intervals since last levodopa dose between the NR (−25.85±62.86 min) and placebo (−2.85±98.77 min) groups. This analysis revealed still a significant decrease in total MDS-UPDRS in the NR group (V1: 46.62±17.76 vs V7: 35.75±11.74; mean change −10.87±11.15; paired t-test, p=0.028) but not in the placebo group (FIG. 7F). We, therefore, deem it unlikely that the difference in the levodopa dose time intervals had a major influence on the observed clinical effects.

### High Dose NR Treatment Augments the NAD Metabolome.

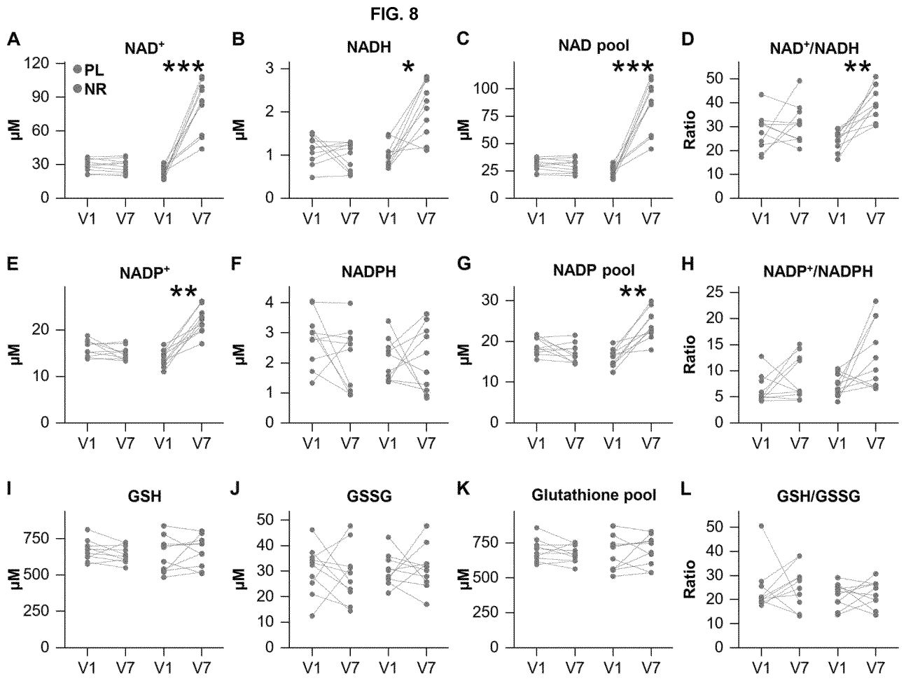

Next, we explored the effect of 3000 mg NR daily on the NAD metabolome in blood and urine. First, flash frozen whole blood samples were analyzed for NAD⁺, NADH, NADP⁺, NADPH and the reduced (GSH) and oxidized (GSSG) forms of glutathione, using the NADMed assay (see methods). The NR group showed a marked increase in the levels of NAD⁺ and NADH, which resulted in an overall elevated NAD⁺/NADH ratio (FIG. 8A-D). Interestingly, we also observed an increase in NADP⁺ and total NADP levels, but not NADPH, and a resulting trend towards increased NADP⁺/NADPH ratio (p=0.056, FIG. 8E-H). In contrast, GSH and GSSG remained unchanged in both groups (FIG. 8I-L).

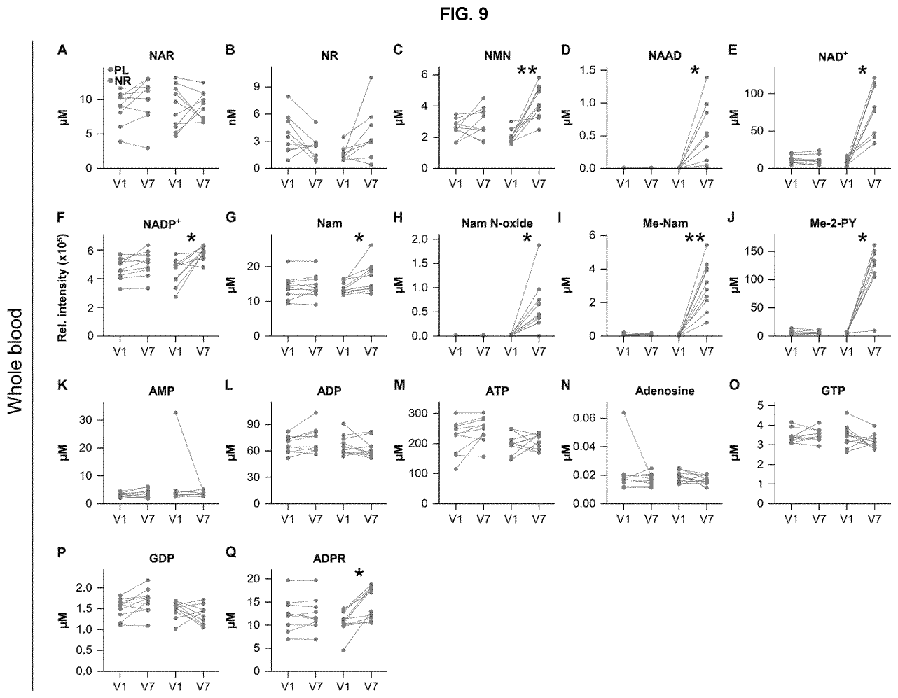

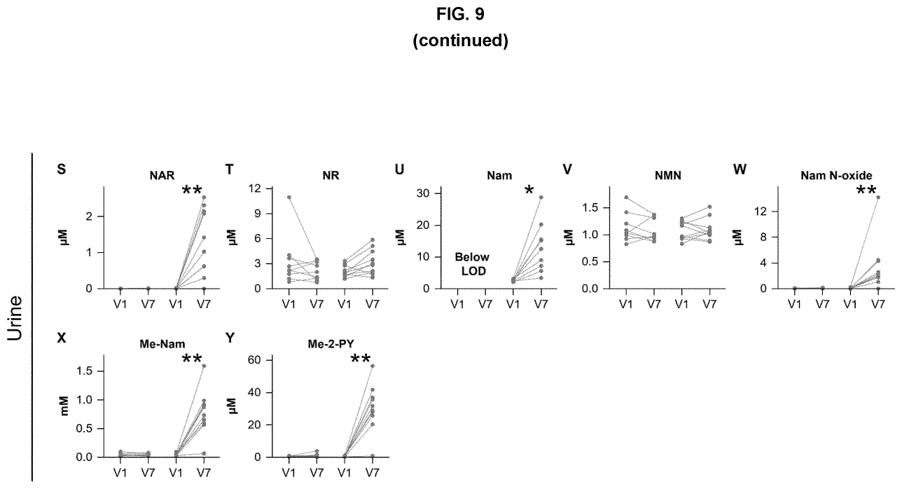

Blood samples were also subjected to targeted LC-MS metabolomics analyses, with a focus on NAD-related metabolites, as well as untargeted metabolomics analyses (see methods). The NR group exhibited a significant increase in whole blood NAD⁺ and NADP⁺ levels, confirming the findings of the NADMed assay (FIG. 9E-F). Multiple metabolites involved in NAD biosynthesis and metabolism were increased as well, including nicotinamide (Nam) and its breakdown products nicotinamide N-oxide (Nam N-oxide), 1-methyl nicotinamide (Me-Nam), and N1-methyl-2-pyridone-5-carboxamide (Me-2-PY; FIG. 9G-J). Nicotinic acid adenine dinucleotide (NAAD) was also elevated as consistently observed in NR-supplementation studies. Interestingly, we also detected elevated levels of nicotinamide mononucleotide (NMN; FIG. 9D). Furthermore, the NR group showed an increase in NAD-derived signaling molecules such as ADP-ribose (ADPR) (FIG. 9Q). In contrast, no significant changes were seen in the placebo group for any of the metabolites. The mean of the individual changes (Δ=V7−V1) in each of these metabolites was also significantly different between the NR and placebo groups, with the exception of NADP⁺, which, however, showed a strong trend after multiple testing correction (p=0.054). ATP and GTP levels were unchanged in both groups, as were ADP, AMP, adenosine and GDP, similar to previous observations (FIG. 9K-P)². NR itself did not increase in the NR group (p=0.129), but the mean of the individual changes between the placebo and NR groups was significantly different (p=0.027). In urine, the NR group exhibited an increase in several NAD-related metabolites, including NAR, Nam and the Nam breakdown products Me-Nam, Me-2-PY and Nam N-oxide, while no significant changes were seen in the placebo group (FIG. 9S-Y). Also here, the mean of the individual changes (Δ=V7−V1) in each metabolite was significantly different between the NR and placebo groups. Similar to blood, NR itself did not change significantly in the NR group (p=0.061), but again, the difference of the means of the individual changes between placebo and NR groups reached significance (p=0.03). Coenzyme A (CoA), Acetyl-CoA and O-acetyl-ADP-ribose were also tested, but were below the limit of detection in the applied method. 2 Brakedal, B. et al. The NADPARK study: A randomized phase I trial of nicotinamide riboside supplementation in Parkinson's disease. Cell Metabolism 34, 396-407.e6 (2022).

High Dose NR Treatment is not Associated with Depletion of the Methyl Group Pool.

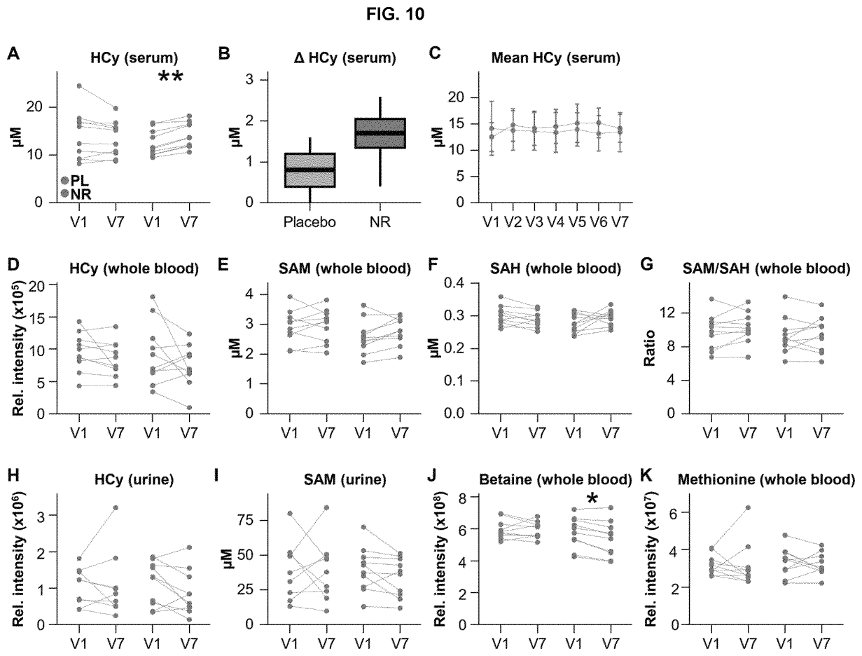

One re-occurring concern in the literature is that NAD precursor supplementation may lead to methyl group depletion due to an increased elimination of Nam by methylation³. To investigate this possibility, we assessed related metabolites, including homocysteine (HCy), S-adenosyl-methionine (SAM) and S-adenosyl-homocysteine (SAH), as well as methionine and betaine. Clinical routine blood assays showed a mild, but significant increase in serum homocysteine levels in the NR group (Δ=+1.66±0.63 μmol/L; p=5.4×10⁻⁴), but not in the placebo group (Δ=−0.73±1.7 μmol/L; p=0.869), when comparing the last visit to baseline (FIG. 10A-B). A more detailed analysis including data from all study visits (V1-V7) indicated that serum HCy only increased already at the second visit (V2, at day 3 of treatment; Δ=+2.29±1.72 μmol/L, paired t-test, p=0.002), and subsequently remained stable until the end of the study (Δ=−0.63±1.76 μmol/L, paired t-test p=0.28; FIG. 10C). For most participants, HCy levels remained within normal range. Three participants had slightly elevated HCy levels already at baseline, while one participant's HCy levels rose from normal to slightly elevated levels. No participants developed any clinical symptoms related to the observed HCy increase. Moreover, serum levels of methylation-relevant metabolites such as folic acid, methylmalonic acid and vitamin B12 remained unchanged. 3 Hwang, E. S. & Song, S. B. Possible Adverse Effects of High-Dose Nicotinamide: Mechanisms and Safety Assessment. Biomolecules 10, 687 (2020).

Moreover, targeted metabolomic analysis of whole blood did not show any HCy elevation (FIG. 10D), or any changes in the major methyl-group donor S-adenosyl-methionine (SAM), or its immediate demethylation product S-adenosyl-homocysteine (SAH; FIG. 10E-G).

Finally, untargeted metabolomic analysis showed that whole blood methionine levels (a precursor for SAM synthesis) were unaffected, while betaine, a substrate for betaine homocysteine methyltransferase catalyzing one way of methionine synthesis, decreased slightly in the NR group (FIG. 10J-K).

### Discussion

Our study met its primary outcome and established that orally administered NR at a dose of 3000 mg daily is safe and well-tolerated in PD over a course of 4 weeks. NR-recipients developed no moderate or severe adverse events. Furthermore, there were no mild adverse events likely attributed to NR, and no other clinical or biochemical signs of toxicity. While our data does not guarantee long-term safety, it allows future dose-optimization and efficacy studies to extend the tested dose range of NR to 3000 mg daily, provided that appropriate safety monitoring is implemented. Furthermore, our results suggest that NR doses up to 3000 mg daily can be initiated directly in NR-naïve individuals.

NR-treatment significantly improved clinical symptoms of PD, measured by MDS-UPDRS, while this remained unchanged in the placebo group. This improvement was driven mostly by MDS-UPDRS part III, which assesses motor function, and part I, which assesses non-motor aspects of daily living. Interestingly, a qualitatively similar but weaker effect was seen in the NADPARK trial where participants consumed 1000 mg NR daily⁴. Taken together, these observations suggest that NAD augmentation may have a symptomatic anti-Parkinson effect. Notably, the treatment group in the present study also exhibited a shorter time from last dose of dopaminergic medication at the final visit compared to baseline. While we cannot confidently exclude that this may have influenced the change in the MDS-UDPRS, it is unlikely to have played a major role for several reasons. First, all participants were examined in the ON-phase, and reported no wearing off. Second, the difference between the groups in time from last dose of dopaminergic medication was small and therefore unlikely to have had a significant clinical effect. Finally, a clinical effect was still evident when comparing a subgroup with similar time from last dose of dopaminergic medication. Nevertheless, it should be stressed that this part of the study was designed to detect potential worsening of the parkinsonism, and not to assess symptomatic improvement. 4 Brakedal, B. et al. The NADPARK study: A randomized phase I trial of nicotinamide riboside supplementation in Parkinson's disease. Cell Metabolism 34, 396-407.e6 (2022).

At a dose of 3000 mg daily, NR treatment greatly augmented the NAD-metabolome, including a ˜3.7-fold (range 1.8-5.8-fold) increase in whole blood NAD⁺ levels. This increase is considerably higher compared to that reported with 1000 mg or 2000 mg NR daily, which increased blood NAD⁺ levels 1.37-2.8-fold. However, in addition to the applied NR dose these studies also vary in terms of both sample type (e.g., whole blood, peripheral blood mononuclear cells or serum), and duration of exposure. Therefore, while providing an indication of a dose effect, the results of these studies are not fully comparable.

In addition to prominently elevated NAD⁺ levels, the NR-group exhibited a clear increase in the NAD⁺/NADH ratio, which can be decreased in PD. Furthermore, NR supplementation was associated with an increase in whole blood NADP⁺. An elevated NADP pool may be beneficial in several ways. NADP is a major redox factor in anabolic synthesis reactions, but also mediates cellular defense against oxidative stress, is involved in cytochrome P450 dependent detoxification mechanisms, and plays a role in immune response by generating oxidative bursts, among others. It is possible that NADP augmentation may contribute to NR-associated neuroprotective effects in PD.

Finally, the observed increase in ADPR further supports a functional impact of NR-induced NAD augmentation on human metabolism.

NR treatment was associated with a mild but significant increase in serum HCy levels. The observed increase in serum HCy levels in our data occurred quickly, during the first three days following commencement of treatment, and levels remained stable thereafter, suggesting that a new equilibrium had been reached. Interestingly, the increase in HCy was only observed in routine clinical serum biochemistry, and not reproduced in whole blood metabolomics by LC-MS. The reason for this may be related to the sample material (i.e., serum vs whole blood), or to method sensitivity. Notably, the change observed in the serum, although statistically significant, was very mild. The observed increase in serum HCy is not unexpected. The NR-induced augmentation of NAD-metabolism leads to increased production of Nam. This is in turn converted to methyl-nicotinamide (Me-Nam) by the action of nicotinamide N-methyltransferase (NNMT), which transfers a methyl group from the universal methyl-donor S-adenosyl methionine (SAM) to Nam, also producing S-adenosylhomocysteine (SAH). SAH is then further converted to homocysteine as part of the homocysteine-methionine cycle (FIG. 11)⁵. Because of the dependence of Nam breakdown on methyl-group donation from SAM, it has been postulated that high dose NAD precursor supplementation may, in theory, lead to overconsumption of SAM and depletion of the methyl-group pool, which could result in an impairment of methylation homeostasis⁶. As we did not detect a change in HCy, SAM, SAH, the SAM/SAH ratio or methionine in whole blood, our data indicates that NR even at a dose of 3000 mg for 4 weeks does not cause depletion of the methyl-group pool. Slightly decreased levels of betaine, a substrate of one of two possible pathways for the re-conversion of HCy to methionine⁷, were observed in the NR group, however, this did affect methionine levels. It is possible that this betaine reduction indicates the higher flux for resynthesis of methionine, and, eventually, SAM, due to the higher demand of methyl group for the methylation of Nam. 5 Komatsu, M. et al. NNMT activation can contribute to the development of fatty liver disease by modulating the NAD+metabolism. Scientific Reports 8, 8637 (2018).6 Hwang, E. S. & Song, S. B. Possible Adverse Effects of High-Dose Nicotinamide: Mechanisms and Safety Assessment. Biomolecules 10, 687 (2020).7 Rizzo, G. & Lagans, A. S. The Link between Homocysteine and Omega-3 Polyunsaturated Fatty Acid: Critical Appraisal and Future Directions. Biomolecules 10, 219 (2020).

It has also been reported that NR treatment at lower doses, e.g., in combination with pterostilbene, may be associated with a mild increase in triglycerides, total cholesterol and LDL⁸. We observed no such effects in our study. In fact, the NR group showed a nominal trend for a reduction in serum triglyceride levels. No trend for increased systolic blood pressure was corroborated by our findings. 8 Dellinger, R. W. et al. Repeat dose NRPT (nicotinamide riboside and pterostilbene) increases NAD+levels in humans safely and sustainably: a randomized, double-blind, placebo-controlled study. NPJ Aging Mech Dis 3, 17 (2017).

### Methods and Materials

### Participants and Study Design

This study design was a single-center, randomized, double-blinded, placebo-controlled trial. The trial was conducted at the Department of Neurology, Haukeland University Hospital, Norway. Inclusion criteria were:
(i) age equal to or greater than 35 years and lower than 100 years at time of enrollment, (ii) clinical diagnosis of idiopathic PD according to the MDS criteria^{9,10}, and (iii) Hoehn and Yahr score <4 at time of enrollment¹¹. Exclusion criteria were: (i) Dementia or other neurodegenerative disorders at baseline visit, (ii) any psychiatric disorder that would interfere with compliance in the study, (iii) any severe somatic illness that would make the individual unable to comply and participate in the study, (iv) use of vitamin B3 supplementation within 30 days prior to enrollment, and (v) metabolic, neoplastic or other physically or mentally debilitating disorders at baseline visit. Patient recruitment, inclusion, and follow-up was carried out by a GCP certified investigator. 9 Postuma, R. B. et al. MDS clinical diagnostic criteria for Parkinson's disease. Mov Disord 30, 1591-1601 (2015).10 Postuma, R. B. & Berg, D. The New Diagnostic Criteria for Parkinson's Disease. Int Rev Neurobiol 132, 55-78(2017).11 Goetz, C. G. et al. Movement Disorder Society Task Force report on the Hoehn and Yahr staging scale: status and recommendations. Mov Disord 19, 1020-1028 (2004).

### Randomization and Masking

20 participants were randomly allocated to receive either 3000 mg NR daily or placebo for 4 weeks (Block size 4, allocation: 1:1), using the electronic Case Report Form (www.viedoc.com). The drug containers were sequentially marked by an independent third party at the research and development department at Haukeland University Hospital. The study drug nicotinamide riboside chloride (NR; Tru Niagen) and placebo were provided by ChromaDex, USA. Each NR-capsule contained 250 mg of NR. Placebo capsules contained microcrystalline cellulose. The NR and placebo capsules were identical in flavor, color, smell and shape. Drug compliance was determined by self-reporting from participants at study visits and a pill count of remaining medication when providing new study medication and at the end of the study. The Center for Clinical Research, Haukeland University Hospital provided allocation sequence, packaging and labelling of drugs in participant-specific kits. Participants, examining physicians as well as medical- and research staff were blinded for the duration of the trial. The sample size was estimated at 10 participants in each group, based on the homogenous response to NR seen in our previous study¹². 12 Brakedal, B. et al. The NADPARK study: A randomized phase I trial of nicotinamide riboside supplementation in Parkinson's disease. Cell Metabolism 34, 396-407.e6 (2022).

### Primary Outcome

The primary outcome of this study was to determine the safety of 3000 mg NR daily for 4 weeks, defined as the absence of clinically significant, NR-associated moderate or severe adverse events.

### Secondary Outcomes

The secondary outcomes were to assess short-term tolerability of 3000 mg NR daily and to determine whether 3000 mg NR causes changes to the NAD metabolome and related metabolites.

### Procedures

At screening for trial entry, candidates underwent physical and neurological examination. Enrolled participants were assessed by physical visits by a movement disorder specialist at baseline (visit 1, V1), day 7 (V4), day 14 (V5), day 21 (V6) and day 28 (V7). At baseline and day 28 participants underwent additional assessment with the Movement Disorder Society Unified Parkinson's disease Rating Scale section I-IV (MDS-UPDRS). At all physical visits an electrocardiogram (ECG), vital parameters (blood pressure, pulse, weight, height) and blood samples were obtained. Additional blood samples were taken on day 3 (V2) and day 5 (V3) to assess safety. The participants were interviewed by telephone consultation by a study nurse on day 3 and day 35 to screen for additional adverse events. Participants were instructed to take their anti-Parkinson drugs as they normally would and no changes were made to anti-Parkinson drug treatment during the trial. Adverse events (AEs) were recorded by the investigating physician. These were then classified by severity according to the Common Terminology Criteria for Adverse Events v5.0 (CTCAE)¹³ as either mild (1), moderate (2), severe (3), life-threatening (4) or death (5). The AE relation to the study drug was scored as unrelated (1), unlikely (2), possible (3), probable (4) or definitely (5). 13 Freites-Martinez, A., Santana, N., Arias-Santiago, S. & Viera, A. Using the Common Terminology Criteria for Adverse Events (CTCAE—Version 5.0) to Evaluate the Severity of Adverse Events of Anticancer Therapies. Actas Dermosifiliogr (Engl Ed) 112, 90-92 (2021).

### Clinical Laboratory Values

Analysis of clinical laboratory values from blood samples was performed by the Department of Medical Biochemistry and Pharmacology (MBF) at Haukeland University Hospital, Bergen, Norway.

NAD and Glutathione Metabolite Analysis from Frozen Blood

Analysis was carried out by NADMed (Helsinki, Finland; www.nadmed.com). NAD and glutathione metabolites were extracted from frozen blood in a single step using a proprietary extraction procedure, and each metabolite was measured individually using optimized cyclic enzymatic assays with colorimetric detection.

LC-MS Analysis from Whole Blood and Urine

Sample analysis was carried out by MS-Omics (Vedbok, Denmark) as follows: Polar metabolite profiling of whole blood and urine was performed using a Thermo Scientific Vanquish LC coupled to Thermo Q Exactive HF MS. An electrospray ionization interface was used as ionization source. Analysis was performed in negative and positive ionization mode. The UPLC was performed using a slightly modified version of the protocol described by Hsiao et al. 2018¹⁴. Peak areas were extracted using Compound Discoverer 3.3 (Thermo Scientific). 14 Hsiao, J. J., Potter, O. G., Chu, T. W. & Yin, H. Improved LC/MS Methods for the Analysis of Metal-Sensitive Analytes Using Medronic Acid as a Mobile Phase Additive. Anal Chem 90, 9457-9464 (2018).

Semi-polar metabolite analysis of whole blood was carried out using a Thermo Scientific Vanquish LC coupled to Orbitrap Exploris 240 MS, Thermo Fisher Scientific. An electrospray ionization interface was used as ionization source. Analysis was performed in positive and negative ionization mode under polarity switching. The UPLC was performed using a slightly modified version of the protocol described by Doneanu et al., 2011¹⁵. Peak areas were extracted using Compound Discoverer 3.3 (Thermo Scientific). 15 Doneanu, C. E. UPLC/MS Monitoring of Water-Soluble Vitamin Bs in Cell Culture Media in Minutes (Application note). https://www.waters.com/waters/library.htm?locale=en US&lid=134636355 (2011).

Identification of compounds for both methods was performed at four levels; Level 1: identification by retention times (compared against in-house authentic standards), accurate mass (with an accepted deviation of 3 ppm), and MS/MS spectra; Level 2a: identification by retention times (compared against in-house authentic standards), accurate mass (with an accepted deviation of 3 ppm). Level 2b: identification by accurate mass (with an accepted deviation of 3 ppm), and MS/MS spectra, Level 3: identification by accurate mass alone (with an accepted deviation of 3 ppm).

### Statistical Analysis

Data normality was tested by the Shapiro-Wilk test. The majority of variables in the dataset were normally distributed. Statistical comparison of continuous variables between baseline and end of study were conducted by two-tailed paired t-tests. Comparison of mean changes between the NR and placebo groups were performed by independent two-tailed t-tests. For values that were not normally distributed, paired and independent Wilcoxon tests were performed. Analysis of categorical variables was carried out with the Fisher's exact test. Correlations were calculated using Pearson's correlation. Multiple testing correction was performed with the Benjamini-Hochberg method. All statistical analyses were performed using R (https://cran.r-project.org, Version 4.2.2, R Foundation for Statistical Computing, Vienna, Austria).

TABLE 6
Demographic and clinical parameters at baseline (V1).

| | Placebo | NR | p- |
|---|---|---|---|
| Parameter | (±sd) | (±sd) | value* |
| Sex (female/male) | 3/7 | 2/8 | 1 |
| Age (years) | 65.5 ± 9 | 61.4 ± 9.28 | 0.33 |
| Height (cm) | 173.9 ± 8.01 | 177 ± 7.75 | 0.39 |
| Weight (kg) | 81.19 ± 10.90 | 79.22 ± 11.26 | 0.7 |
| Time since PD | 6.9 ± 4.15 | 5.4 ± 2.91 | 0.37 |
| diagnosis (years) | | | |
| BMI (kg/m²) | 26.79 ± 2.53 | 25.24 ± 2.86 | 0.22 |
| Hoehn & Yahr | 1.9 ± 0.32 | 2 ± 0.67 | 0.73 |
| Total MDS-UPDRS | 41.8 ± 16.46 | 51 ± 21.28 | 0.29 |
| MDS-UPDRS Part I | 6.6 ± 3.92 | 7.8 ± 5.37 | 0.58 |
| MDS-UPDRS Part II | 8.2 ± 2.74 | 9.4 ± 6.25 | 0.59 |
| MDS-UPDRS Part III | 24.7 ± 11.95 | 29.7 ± 12.85 | 0.38 |
| MDS-UPDRS Part IV | 2.3 ± 2.94 | 4.1 ± 4.88 | 0.33 |

*Fisher's exact test was used for comparing sex ratios. The remaining parameters were calculated using independent t-test for normally distributed data and independent Wilcoxon test for non-normally distributed data.

TABLE 7
Adverse events.

| | | | Causal | | CTCAEⁱ |
|---|---|---|---|---|---|
| | | | relationship | p- | grading |
| Adverse event | NR | Placebo | to study drug (n) | value* | Grade 1-5 (n) |
| Headache | 3 | 0 | Possible (1) | 0.21 | Grade 1 (3) |
| | | | Unlikely (2) | | |
| Dyspepsia | 2 | 1 | Possible (3) | 1 | Grade 1 (3) |
| Nausea | 2 | 0 | Possible (2) | 0.47 | Grade 1 (2) |
| Dizziness | 1 | 1 | Possible (2) | 1 | Grade 1 (2) |
| Fatigue | 2 | 1 | Unlikely (2) | 1 | Grade 1 (3) |
| | | | Possible (1) | | |
| Dry mouth | 1 | 0 | Possible (1) | 1 | Grade 1 (1) |
| Abdominal pain | 1 | 3 | Probable (1) | 0.58 | Grade 1 (4) |
| | | | Possible (2) | | |
| | | | Unlikely (1) | | |
| Rash maculo-papular | 1 | 1 | Unlikely (2) | 1 | Grade 1 (2) |
| Insomnia | 1 | 0 | Unlikely (1) | 1 | Grade 1 (1) |
| Upper respiratory infection | 1 | 1 | Unlikely (2) | 1 | Grade 1 (2) |
| Urinary frequency | 1 | 0 | Unlikely (1) | 1 | Grade 1 (1) |
| Muscle cramp^{a,b} | 2 | 0 | Possible (1) | 0.47 | Grade 1 (2) |
| | | | Unlikely (1) | | |
| Chest pain - cardiac | 0 | 1 | Unlikely (1) | 1 | Grade 1 (1) |
| Tremor^{c} | 2 | 2 | Possible (4) | 1 | Grade 1 (4) |
| Localized edema | 0 | 1 | Unlikely (1) | 1 | Grade 1 (1) |
| Extrapyramidal disorder^{d,e,f,g} | 3 | 1 | Possible (4) | 0.58 | Grade 1 (4) |
| Gastrointestinal disorders - other - | 1 | 1 | Possible (1) | 1 | Grade 1 (2) |
| abdominal gas discomfort | | | Probable (1) | | |
| Gastrointestinal disorders - other - | 0 | 1 | Possible (1) | 1 | Grade 1 (1) |
| increased salivation | | | | | |
| Metabolism and nutrition disorders - | 0 | 1 | Possible (1) | 1 | Grade 1 (1) |
| Decrease in blood glucose within | | | | | |
| normal range^{h} | | | | | |
| Injury, poisoning and procedural | 1 | 0 | Unrelated (1) | 1 | Grade 1 (1) |
| complications - Contusion of ankle | | | | | |
| Injury, poisoning and procedural | 0 | 1 | Unlikely (1) | 1 | Grade 1 (1) |
| complications - Mild head trauma | | | | | |

*Fisher's exact test.
^{a}Multiple adverse events reported by one subject.
^{b}Former medical history of dystonia. Increased muscle cramps one week after cessation of the study drug.
^{c}One patient in each treatment group reported increased tremor in the first week after cessation of study drug. The other two reported increased tremor during study drug administration.
^{d}One participant (NR) reported increased dyskinesia during the study, causing the participant to auto-reduce levodopa dosage.
^{e}One participant (placebo) reported decreased dyskinesia during study.
^{f}One participant (NR) reported increased rigidity one week after study drug cessation.
^{g}One participant (NR) auto-reduced their levodopa dose during the study due to reduced subjective need of medication.
^{h}Caused diabetic participant to auto-reduce insulin dosage.
ⁱCommon Terminology Criteria for Adverse Events v5.0.

TABLE 8
Vital signs and body metrics.

| | | Placebo | NR | p- |
|---|---|---|---|---|
| Parameter | Visit | (±sd) | (±sd) | value* |
| Diastolic blood | V1 | 78.3 ± 11.78 | 74.7 ± 8.49 | 0.34 |
| pressure (mmHg) | V7 | 73.1 ± 4.30 | 76.2 ± 6.81 | |
| | Δ | −5.2 ± 12.58 | 1.5 ± 8.31 | |
| | p-value** | 0.31 | 1 | |
| Pulse (min⁻¹) | V1 | 67.8 ± 7.71 | 64.2 ± 14.67 | 0.17 |
| | V7 | 71.4 ± 9.91 | 61.1 ± 12.95 | |
| | Δ | 3.6 ± 10.18 | −3.1 ± 9.48 | |
| | p-value** | 0.35 | 0.52 | |
| Systolic blood | V1 | 131.4 ± 21.46 | 122.1 ± 12.53 | 0.23 |
| pressure (mmHg) | V7 | 121.6 ± 7.97 | 122.2 ± 7.49 | |
| | Δ | −9.8 ± 23.25 | 0.1 ± 8.29 | |
| | p-value** | 0.21 | 0.97 | |
| Weight (kg) | V1 | 81.19 ± 10.90 | 79.38 ± 11.92 | 0.20 |
| | V7 | 80.73 ± 10.79 | 79.38 ± 11.79 | |
| | Δ | −0.46 ± 0.9 | 0 ± 0.58 | |
| | p-value** | 0.14 | 1 | |

*P-values are comparisons of Δ values between the NR and placebo group.
**P-value are comparisons between V7 and V1 in each treatment group. For comparison within one group, paired two-tailed t-test or Wilcoxon test was used. For comparison between treatment groups, independent two-tailed t-test or Wilcoxon test was used. Non-normally distributed data was analyzed by Wilcoxon test.

TABLE 9
MDS-UPDRS, Hoehn and Yahr scores and time since levodopa.

| | | Placebo | NR | p- |
|---|---|---|---|---|
| Parameter | Visit | (±sd) | (±sd) | value* |
| Total MDS- | V1 | 41.8 ± 16.46 | 51 ± 21.28 | 0.024 |
| UPDRS | V7 | 41.8 ± 22.23 | 40.3 ± 17.10 | |
| | Δ | 0 ± 9.59 | −10.7 ± 9.94 | |
| | p-value** | 1 | 0.007 | |
| MDS- | V1 | 6.6 ± 3.92 | 7.8 ± 5.37 | 0.171 |
| UPDRS | V7 | 6.3 ± 4.99 | 5.6 ± 5.01 | |
| Part I | Δ | −0.3 ± 2.26 | −2.2 ± 3.52 | |
| | p-value** | 0.684 | 0.079 | |
| MDS- | V1 | 8.2 ± 2.74 | 9.4 ± 6.25 | 0.93 |
| UPDRS | V7 | 7.5 ± 4.17 | 8.8 ± 7.58 | |
| Part II | Δ | −0.7 ± 3.26 | −0.6 ± 2.36 | |
| | p-value** | 0.515 | 0.443 | |
| MDS- | V1 | 24.7 ± 11.95 | 29.7 ± 12.85 | 0.052 |
| UPDRS | V7 | 24.6 ± 16.46 | 22.7 ± 7.55 | |
| Part III | Δ | −0.1 ± 6.79 | −7 ± 7.98 | |
| | p-value** | 0.963 | 0.021 | |
| MDS- | V1 | 2.3 ± 2.94 | 4.1 ± 4.88 | 0.087 |
| UPDRS | V7 | 3.4 ± 3.59 | 3.2 ± 3.15 | |
| Part IV | Δ | 1.1 ± 2.33 | −0.9 ± 2.60 | |
| | p-value** | 0.169 | 0.302 | |
| Hoehn & | V1 | 1.9 ± 0.31 | 2 ± 0.66 | 0.754 |
| Yahr score | V7 | 1.9 ± 0.56 | 2.1 ± 0.31 | |
| | Δ | 0 ± 0.66 | 0.1 ± 0.56 | |
| | p-value** | 1 | 0.772 | |
| Time since | V1 | 89 ± 79.25 | 154.33 ± 88.51 | 0.090 |
| levodopa | V7 | 117.33 ± 31.71 | 105.33 ± 61.60 | |
| (min) | Δ | 28.33 ± 105.58 | −49 ± 71.925 | |
| | p-value** | 0.444 | 0.075 | |

*P-values are comparisons of Δ values between the NR and placebo group.
**P-value are comparisons between V7 and V1 in each treatment group. For comparison within one group, paired two-tailed t-test or Wilcoxon test was used. For comparison between treatment groups, independent two-tailed t-test or Wilcoxon test was used. Non-normally distributed data was analyzed by Wilcoxon test.

### Example 4: NAD-Brain: A Pharmacokinetic Study of Brain NAD Replenishment Therapy

### 1. Protocol Synopsis

| Study objective | Determine the blood and brain pharmacokinetics of NAD replenishment | |
|---|---|---|
| | therapy (NRT) with NR and NMN | |
| Phase and study | Phase I, open label | |
| type | | |
| Investigational | Nicotinamide Riboside (NR), Nicotinamide Mononucleotide (NMN) | |
| Product (IP) | | |
| Treatment | Single doses/days (see protocol) | |
| Duration: | | |
| Objectives | Primary Objective: | |
| | To determine the change over 20 days in the blood NAD-metabolome and | |
| | cerebral NAD levels, following the administration of oral NAD replenishment | |
| | therapy (NRT) with the following NAD precursors: NR 600 mg × 2 daily, NMN | |
| | 600 mg × 2 daily. | |
| | Secondary objectives: | |
| | 1) | Determine the optimal dosing intervals for different doses of NRT, |
| | | to maintain a stable increase in cerebral and blood NAD-levels |
| | | during treatment. |
| | 2) | Assess whether there are interindividual differences in the time |
| | | course of change in the blood NAD-metabolome and cerebral NAD |
| | | increase, following the administration of oral NRT. |
| | 3) | Assess whether there are sex-dependent differences in the time |
| | | course of change in the blood NAD-metabolome and cerebral NAD |
| | | increase, following the administration of oral NRT. |
| Outcomes: | Primary Outcomes | |
| | The change of cerebral NAD levels (measured by 31P-MRS) and of blood | |
| | NAD-metabolites (measured by HPLC-MS), over time (20 days), after the | |
| | administration of oral NRT with the following NAD precursors: NR 600 mg × 2 | |
| | daily, NMN 600 mg × 2 daily. | |
| | Secondary outcomes: | |
| | 1) | Descriptive analyses of interindividual differences in the time |
| | | course of change in the blood NAD-metabolome and cerebral NAD |
| | | increase, following the administration of oral NRT. |
| | 2) | Between-sex differences in the time course of change in the blood |
| | | NAD-metabolome and cerebral NAD increase, following the |
| | | administration of oral NRT. |
| Study Design: | The NAD-brain study is a parallel assessment of NRT pharmacokinetics in | |
| | the blood and brain of healthy human subjects and subjects with | |
| | Parkinson's disease (PD). Healthy individuals and individuals with PD | |
| | undergo repeated blood sampling and 31P-MRS brain scans during two 20- | |
| | day periods, each of which starts with 8 days of daily intake of NR 600 mg × | |
| | 2, or NMN 600 mg × 2. The two 20-day periods are 14 days apart to allow | |
| | for washout of the previous compound. Blood is analyzed for NR and NAD- | |
| | metabolites using HPLC-MS. By this approach we, measure the | |
| | simultaneous change in NAD-metabolism over time in blood and brain and | |
| | establish blood and brain pharmacokinetics for NRT in humans. These | |
| | results allow determining the optimal dosing frequency of NRT in healthy | |
| | individuals and individuals with PD. | |
| Main Inclusion | For healthy individuals: | |
| Criteria: | | Age 30-85 years at the time of enrollment. |
| | | Neurologically healthy at the time of enrollment. |
| | For individuals with PD: | |
| | | Clinical diagnosis of idiopathic PD according to the MDS criteria. |
| | | 123I-loflupane dopamine transporter imaging (DAT-scan) confirming |
| | | nigrostriatal degeneration. |
| | | Hoehn and Yahr score <4 at enrollment. |
| | | Age 50-85 years at the time of enrollment. |
| Main Exclusion | For healthy individuals | |
| Criteria | | History of acute or chronic neurological disorder affecting the central |
| | | nervous system (CNS). |
| | | Migraine, cluster headache, and tension headache are |
| | | allowed, but not on the day of the study visits. |
| | | Impaired renal function. |
| | | Impaired hepatic function. |
| | | Severe hematological disease. |
| | | Any psychiatric disorder that would interfere with compliance in the |
| | | study. |
| | | Any severe somatic illness that would make the individual unable to |
| | | comply and participate in the study. |
| | | Mitochondrial disease. |
| | | Use of high dose vitamin B3 supplementation within 30 days of |
| | | enrolment. |

For individuals with PD:

```
Dementia or other neurodegenerative disorder at baseline visit.
Diagnosed with atypical parkinsonism (PSP, MSA, CBD) or vascular
parkinsonism.
History of acute or chronic neurological disorder, other than PD,
affecting the central nervous system (CNS).
  Migraine, cluster headache, and tension headache are
  allowed, but not on the day of the study visits.
Impaired renal function.
Impaired hepatic function.
Severe hematological disease.
Any psychiatric disorder that would interfere with compliance in the
study.
Any severe somatic illness that would make the individual unable to
comply and participate in the study.
Mitochondrial disease.
Use of high dose vitamin B3 supplementation within 30 days of
enrolment.
```

| | |
|---|---|
| Efficacy | Brain NAD-levels, measured by 31P-MRS and blood NAD-metabolome, |
| Assessments: | measured by HPLC-MS and the NADmed assay. |

### 2. Rationale for the Study and Purpose

To further develop the potential of NAD replenishment therapy towards a neuroprotective therapy for PD, we need to determine the optimal dosing regimen, including dose size and frequency. The NAD-brain study aims at determining the optimal dosing regimen by performing a parallel assessment of NAD replenishment therapy pharmacokinetics in the blood and brain of healthy human subjects and subjects with Parkinson's disease (PD).

An optimal dosing regimen of NR would result in constant increase in brain NAD levels over time. There has been a demand for adequate pharmacokinetic data to determine this. A study in a single subject showed that blood NAD levels peaked 7.7 hours after intake of 1000 mg NR, and decreased, but not yet to baseline, 23.8 h after intake¹⁶. Another study showed that blood NAD levels were stably increased in a 24h-period after the intake of 1000 mg NR, in individuals who had been receiving increasing NR doses, given twice daily, for nine days¹⁷. Taken together, available data are not sufficient to provide the full pharmacokinetic profile of NR in blood. Moreover, blood does not necessarily reflect the situation in the brain, due to both peripheral consumption, the presence of the blood brain barrier, and the unknown dynamics of local NR-metabolism in the human brain. Similar to NR, limited pharmacokinetic data from blood are available from NMN¹⁸, but data from the human brain are lacking. 16 Trammell S A, Schmidt M S, Weidemann B J, et al. Nicotinamide riboside is uniquely and orally bioavailable in mice and humans. Nat Commun 2016, 7:1294817 Airhart S E, Shireman L M, Risler L J, et al. An open-label, non-randomized study of the pharmacokinetics of the nutritional supplement nicotinamide riboside (NR) and its effects on blood NAD⁺ levels in healthy volunteers. PloS one 2017; 12(12):e0186459.18 Pencina K M, Lavu S, dos Santos M, et al. MIB-626, an Oral Formulation of a Microcrystalline Unique Polymorph of β-Nicotinamide Mononucleotide, Increases Circulating Nicotinamide Adenine Dinucleotide and its Metabolome in Middle-Aged and Older Adults. The Journals of Gerontology: Series A 2022; glac049.

The NAD-brain study performs a parallel assessment of NAD replenishment therapy pharmacokinetics in the blood and brain of healthy human subjects and subjects with Parkinson's disease (PD). A total of 10 healthy individuals (5 men and 5 women) and 10 individuals with PD (5 men and 5 women) undergo repeated blood sampling and 31P-MRS brain scans during two 20-day periods, each of which starts with 8 days of daily intake of NR 600 mg×2, or NMN 600 mg×2. The two 20-day period is 14 days apart to allow for washout of the previous compound. Blood is analyzed for NR and NAD-metabolites using HPLC-MS. By this approach, we measured the simultaneous change in NAD-metabolism overtime in blood and brain and establish blood and brain pharmacokinetics for NAD replenishment therapy in humans. These results contribute to determining the optimal dosing frequency of NAD replenishment therapy in healthy individuals and individuals with PD.

### 3. Treatment

NR (Niagen®, Chromadex) at a dose of 600 mg×2 daily). NMN (Restorin®, from Seragon Pharmaceutics, or MIB-626 from Metro International Biotech) at a dose of 600 mg×2 daily.

### 4. Study Procedures

At screening, participants are assessed for eligibility, inclusion/exclusion criteria, medical history and concomitant medication is recorded, and informed consent signed.
The NAD-brain study performs a parallel assessment of NAD replenishment therapy pharmacokinetics in the blood and brain of healthy human subjects and subjects with Parkinson's disease (PD). Healthy individuals and individuals with PD undergo repeated blood sampling and 31P-MRS brain scans during two 20-day periods, each of which starts with 8 days of daily intake of NR 600 mg×2, or NMN 600 mg×2 (Table 10).
For each 20-day period the testing (i.e., scanning and blood draw) frequency is shown in Table 10, below. The two 20-day periods are 14 days apart to allow for washout of the previous compound.

By this approach, measuring the simultaneous change in NAD-metabolism over time in blood and brain and establishing blood and brain pharmacokinetics for NAD replenishment therapy in humans is achieved. Based on these results, the optimal dosing frequency of NR in healthy individuals and individuals with PD can be determined.

TABLE 10
Trial flow chart

| | day | | | | | | | | | | | | | | | | | | | |
|---|---|---|---|---|---|---|---|---|---|---|---|---|---|---|---|---|---|---|---|---|
| Timepoint | 0 | 01 | 02 | 03 | 04 | 05 | 06 | 07 | 08 | 09 | 10 | 11 | 12 | 13 | 14 | 15 | 16 | 17 | 18 | 19 |
| 08:00 | | θ | θ | θ | θ | θ | θ | θ | θ | | | | | | | | | | | |
| Ca 15:00 | xb | x | x | x | | x | | | x | x | x | | x | | | x | | | | x |
| 20:00 | θ* | θ | θ | θ | θ | θ | θ | θ | W | W | W | W | W | W | W | W | W | W | W | W |

Θ: dosing.
b: baseline measurement.
x: blood sampling and MRS scan.
W: washout period.
*first dose taken after baseline.

Blood is analyzed for NR and NAD-metabolites using HPLC-MS metabolomics. Snap-frozen whole blood is used for the analysis of NAD⁺. Blood is drawn from the participant in EDTA tube (VACUETTE® TUBE 2 ml K2E K2EDTA). The EDTA tube is gently inverted at least 10 times without shaking; 8×200 μl of blood from the EDTA tube is aliquoted into 8×0.5 ml micro tubes; these are closed, and all the micro tubes are immerse simultaneously in liquid nitrogen within 2 min from when blood has started flowing into the EDTA tube; then transferred frozen to −80° C. freezer for storage; stored at −80C in freezer-compatible cardboard or plastic storage boxes; and shipped on dry ice.

### 5. Analyses & Statistical Approach

### Primary Analysis

The change of cerebral NAD levels (measured by 31P-MRS) and of the levels of blood NAD-metabolites (measured by HPLC-MS), are characterized over a 20-day period. Based on these measurements, the blood and cerebral bioavailability and half-life will be estimated and the optimal dosing regimen for stable brain and blood NAD-levels determined. Descriptive statistics are employed. There is no group comparison.

### Secondary Analyses

Descriptive analyses of interindividual differences in the time course of change in the blood NAD-metabolome and cerebral NAD increase, following the administration of oral NRT. Between-sex differences in the time course of change in the blood NAD-metabolome and cerebral NAD increase, following the administration of oral NRT.

### 6. Results

This is a pharmacokinetic (PK) study where 6 healthy individuals (3 male and 3 female):
1) Received NR 1200 mg daily (600 mg×2) for 8 days, followed by an additional 11 days follow up, while undergoing serial blood and brain NAD-measurement.
2) Received NMN 1200 mg daily (600 mg×2) for 8 days, followed by an additional 11 days follow up, while undergoing serial blood and brain NAD-measurement.

The individuals were mixed, so that in stage-1 three individuals received NR and three individuals received NMN, then in stage-2 they switched (Cross-over design). There were almost three weeks (20 days) of washout between the last dose of the first compound, and before the first dose of the second compounds.

### Blood

In the blood, NAD⁺ levels increased in all individuals. The following key observations were made:
1. The overall NR response was stronger than the NMN response. NR intake produced higher increases in blood NAD⁺ levels, and declined more slowly after the last dose intake.
2. There are interindividual differences in the blood NAD⁺ response, and those seem to be largely consistent between NR and NMN. I.e., the strongest and weakest response was seen in the same two individuals, individual-2 and individual-4 respectively, for both compounds. This suggests that there are individual intrinsic factors determining the magnitude of the NAD⁺ response, following the intake of precursors. Such factors may include (but are not limited to):
   a. Genetics influencing the absorption and metabolism of the precursors and/or NAD⁺ itself.
   b. The composition of the intestinal microbiome.
   c. Dietary factors
3. NAD⁺ levels did not plateau after 8 days of high dose precursor intake (NR or NMN). This was unexpected and it signifies that, for some yet undetermined reason, it takes more than a week to maximally boost NAD metabolism and reach a new steady state. This may also be a reason that the observed brain response is moderate (see next section)

### Brain

In the brain, total NAD (NAD⁺+NADH) levels were measured by 31P-MRS as previously described¹⁹. Blood NAD data exhibited less noise than data from brain measurements. 19 Brakedal, B. et al. The NADPARK study: A randomized phase I trial of nicotinamide riboside supplementation in Parkinson's disease. Cell Metab 34, 396-407.e6 (2022).

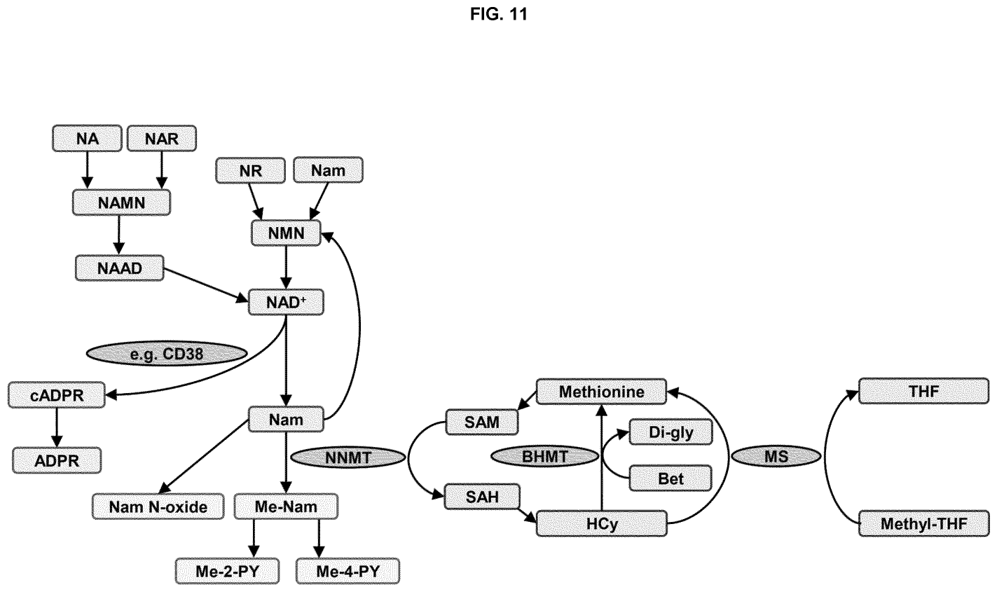

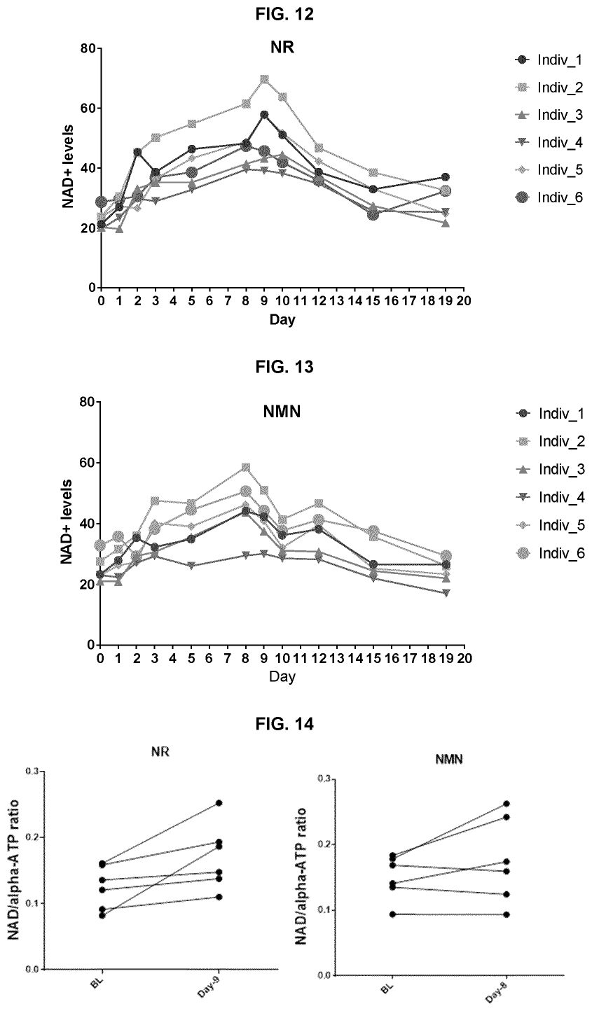

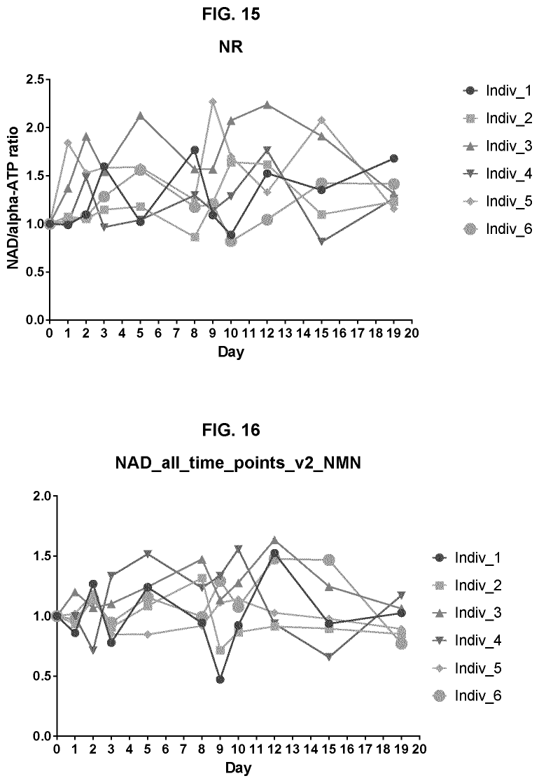

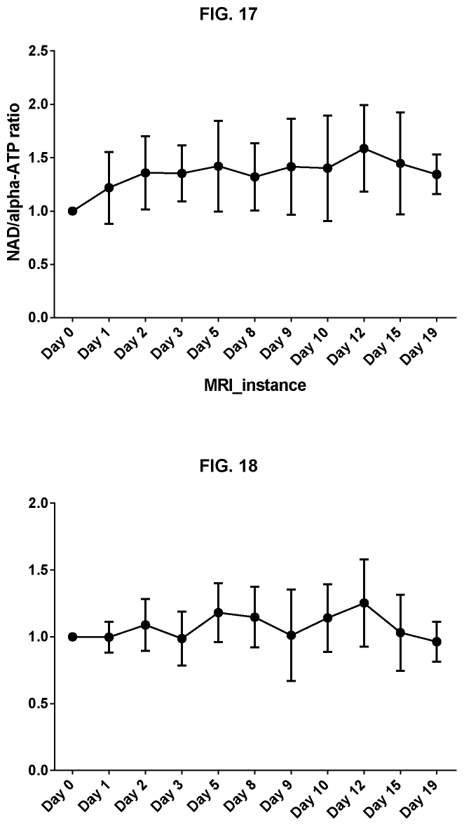

Comparison of baseline to the day of maximal NAD levels in the blood (i.e., day 9 for NR recipients and day 8 for NMN recipients, see FIG. 12, FIG. 13) showed a significant increase in brain NAD levels in NR recipients (p=0.038), but not in NMN recipients (P=0.17) (FIG. 14, FIG. 15). Inspecting all time points shows a trend for an initial increase of brain NAD-levels over time, followed by a decrease after day 12. The data is suitable for assessing the NAD-level increase (FIGS. 15-18). As compared to 31P-MRS, direct blood NAD-measurements appear to be even better suited to follow brain NAD kinetics in real time, from the view point of reduced noise.

Notably, NAD levels returned to baseline by day 19 with NMN, but remained elevated with NR. The blood and brain profile of NR are suggestive of a more sustained response.

The strongest and weakest responders were the same in stage-1 and stage-2 (i.e. with NR or NMN), suggesting this is an intrinsic effect specific to the individual, possibly associated with the person's genetics and/or the microbiome composition.

Based on the observations, a stronger brain NAD-response is considered possible, especially in view of the following:
a. Longer exposure time. Since blood NAD-levels did not plateau after 8 days, longer intake may be required and the brain response may be delayed.
b. Age. Since NAD-levels are generally known to decline with age, a stronger increase may be seen in the brain older individuals.
c. Disease. The NAD-response may depend upon a preexisting aberrant metabolism, so that stronger increases may be observed in the brain of patients, such as with PD.

### DISCUSSION AND CONCLUSIONS

Thus, as demonstrated herein above, nicotinamide riboside is a compound capable of effectively increasing the NAD levels in the brain which can be used as a safe, well-tolerated and beneficial therapy, in particular as neuroprotective treatment, for inhibiting PD progression. Specifically, tolerability and improved efficacy of NR at higher dosages, associated with improved biological and clinical responses, may be achieved by escalating the dose by using the dosage regimens and method steps disclosed herein. Additionally, the present invention allows to account for individual variability in cerebral NAD metabolism and find an individually appropriate dosage for a certain patient, to achieve an optimal neurometabolic response, while maintaining acceptable safety and tolerability, while achieving a multitude of additional beneficial therapeutic effects. The present therefore invention can thus provide an improved, efficient and safe treatment of PD addressing the physiological causes of PD in humans, rather than just for alleviating the disease symptoms.

```
SEQ ID NO. 1: SNCA
(UniprotKB-P37840 (SYUA_HUMAN); HGNC: 11138, SNCA)
                10 20 30 40 50
MDVFMKGLSK AKEGVVAAAE KTKQGVAEAA GKTKEGVLYV GSKTKEGVVH
                60 70 80 90 100
GVATVAEKTK EQVTNVGGAV VTGVTAVAQK TVEGAGSIAA ATGFVKKDQL
              110 120 130 140
GKNEEGAPQE GILEDMPVDP DNEAYEMPSE EGYQDYEPEA
SEQ ID NO. 2: LRRK2
(UniProtKB-Q5S007 (LRRK2_HUMAN); HGNC: 18618)
                10 20 30 40 50
MASGSCQGCE EDEETLKKLI VRLNNVQEGK QIETLVQILE DLLVETYSER
                60 70 80 90 100
ASKLFQGKNI HVPLLIVLDS YMRVASVQQV GWSLLCKLIE VCPGTMQSLM
              110 120 130 140 150
GPQDVGNDWE VLGVHQLILK MLTVHNASVN LSVIGLKTLD LLLTSGKITL
              160 170 180 190 200
LILDEESDIF MLIFDAMHSF PANDEVQKLG CKALHVLFER VSEEQLTEFV
              210 220 230 240 250
ENKDYMILLS ALTNFKDEEE IVLHVLHCLH SLAIPCNNVE VLMSGNVRCY
              260 270 280 290 300
NIVVEAMKAF PMSERIQEVS CCLLHRLTLG NFFNILVLNE VHEFVVKAVQ
              310 320 330 340 350
QYPENAALQI SALSCLALLT ETIFLNQDLE EKNENQENDD EGEEDKLEWL
              360 370 380 390 400
EACYKALTWH RKNKHVQEAA CWALNNLLMY QNSLHEKIGD EDGHFPAHRE
              410 420 430 440 450
VMLSMLMHSS SKEVFQASAN ALSTLLEQNV NERKILLSKG IHLNVLELMQ
              460 470 480 490 500
KHIHSPEVAE SGCKMLNHLF EGSNTSLDIM AAVVPKILTV MKRHETSLPV
              510 520 530 540 550
QLEALRAILH FIVPGMPEES REDTEFHHKL NMVKKQCFKN DIHKLVLAAL
              560 570 580 590 600
NRFIGNPGIQ KCGLKVISSI VHFPDALEML SLEGAMDSVL HTLQMYPDDQ
              610 620 630 640 650
EIQCLGLSLI GYLITKKNVF IGTGHLLAKI LVSSLYRFKD VAEIQTKGFQ
              660 670 680 690 700
TILAILKLSA SFSKLLVHHS FDLVIFHQMS SNIMEQKDQQ FLNLCCKCFA
              710 720 730 740 750
KVAMDDYLKN VMLERACDQN NSIMVECLLL LGADANQAKE GSSLICQVCE
              760 770 780 790 800
KESSPKLVEL LLNSGSREQD VRKALTISIG KGDSQIISLL LRRLALDVAN
              810 820 830 840 850
NSICLGGFCI GKVEPSWLGP LFPDKTSNLR KQTNIASTLA RMVIRYQMKS
              860 870 880 890 900
AVEEGTASGS DGNFSEDVLS KFDEWTFIPD SSMDSVFAQS DDLDSEGSEG
              910 920 930 940 950
SFLVKKKSNS ISVGEFYRDA VLQRCSPNLQ RHSNSLGPIF DHEDLLKRKR
              960 970 980 990 1000
KILSSDDSLR SSKLQSHMRH SDSISSLASE REYITSLDLS ANELRDIDAL
            1010 1020 1030 1040 1050
SQKCCISVHL EHLEKLELHQ NALTSFPQQL CETLKSLTHL DLHSNKFTSF
            1060 1070 1080 1090 1100
PSYLLKMSCI ANLDVSRNDI GPSVVLDPTV KCPTLKQFNL SYNQLSFVPE
            1110 1120 1130 1140 1150
NLTDVVEKLE QLILEGNKIS GICSPLRLKE LKILNLSKNH ISSLSENFLE
            1160 1170 1180 1190 1200
ACPKVESFSA RMNFLAAMPF LPPSMTILKL SQNKFSCIPE AILNLPHLRS
            1210 1220 1230 1240 1250
LDMSSNDIQY LPGPAHWKSL NLRELLESHN QISILDLSEK AYLWSRVEKL
            1260 1270 1280 1290 1300
HLSHNKLKEI PPEIGCLENL TSLDVSYNLE LRSFPNEMGK LSKIWDLPLD
            1310 1320 1330 1340 1350
ELHLNFDFKH IGCKAKDIIR FLQQRLKKAV PYNRMKLMIV GNTGSGKTTL
            1360 1370 1380 1390 1400
LQQLMKTKKS DLGMQSATVG IDVKDWPIQI RDKRKRDLVL NVWDFAGREE
            1410 1420 1430 1440 1450
FYSTHPHFMT QRALYLAVYD LSKGQAEVDA MKPWLFNIKA RASSSPVILV
            1460 1470 1480 1490 1500
GTHLDVSDEK QRKACMSKIT KELLNKRGFP AIRDYHFVNA TEESDALAKL
            1510 1520 1530 1540 1550
RKTIINESLN FKIRDQLVVG QLIPDCYVEL EKIILSERKN VPIEFPVIDR
            1560 1570 1580 1590 1600
KRLLQLVREN QLQLDENELP HAVHELNESG VLLHFQDPAL QLSDLYFVEP
            1610 1620 1630 1640 1650
KWLCKIMAQI LTVKVEGCPK HPKGIISRRD VEKFLSKKRK FPKNYMSQYF
            1660 1670 1680 1690 1700
KLLEKFQIAL PIGEEYLLVP SSLSDHRPVI ELPHCENSEI IIRLYEMPYF
            1710 1720 1730 1740 1750
PMGFWSRLIN RLLEISPYML SGRERALRPN RMYWRQGIYL NWSPEAYCLV
            1760 1770 1780 1790 1800
GSEVLDNHPE SFLKITVPSC RKGCILLGQV VDHIDSLMEE WFPGLLEIDI
            1810 1820 1830 1840 1850
CGEGETLLKK WALYSENDGE EHQKILLDDL MKKAEEGDLL VNPDQPRLTI
            1860 1870 1880 1890 1900
PISQIAPDLI LADLPRNIML NNDELEFEQA PEFLLGDGSF GSVYRAAYEG
            1910 1920 1930 1940 1950
EEVAVKIFNK HTSLRLLRQE LVVLCHLHHP SLISLLAAGI RPRMLVMELA
            1960 1970 1980 1990 2000
SKGSLDRLLQ QDKASLTRTL QHRIALHVAD GLRYLHSAMI IYRDLKPHNV
            2010 2020 2030 2040 2050
LLFTLYPNAA IIAKIADYGI AQYCCRMGIK TSEGTPGFRA PEVARGNVIY
            2060 2070 2080 2090 2100
NQQADVYSFG LLLYDILTTG GRIVEGLKFP NEFDELEIQG KLPDPVKEYG
            2110 2120 2130 2140 2150
CAPWPMVEKL IKQCLKENPQ ERPTSAQVED ILNSAELVCL TRRILLPKNV
            2160 2170 2180 2190 2200
IVECMVATHH NSRNASIWLG CGHTDRGQLS FLDLNTEGYT SEEVADSRIL
            2210 2220 2230 2240 2250
CLALVHLPVE KESWIVSGTQ SGTLLVINTE DGKKRHTLEK MTDSVTCLYC
            2260 2270 2280 2290 2300
NSFSKQSKQK NFLLVGTADG KLAIFEDKTV KLKGAAPLKI LNIGNVSTPL
            2310 2320 2330 2340 2350
MCLSESTNST ERNVMWGGCG TKIFSFSNDF TIQKLIETRT SQLFSYAAFS
            2360 2370 2380 2390 2400
DSNIITVVVD TALYIAKQNS PVVEVWDKKT EKLCGLIDCV HELREVMVKE
            2410 2420 2430 2440 2450
NKESKHKMSY SGRVKTLCLQ KNTALWIGTG GGHILLLDLS TRRLIRVIYN
            2460 2470 2480 2490 2500
FCNSVRVMMT AQLGSLKNVM LVLGYNRKNT EGTQKQKEIQ SCLTVWDINL
            2510 2520
PHEVQNLEKH IEVRKELAEK MRRTSVE
SEQ ID NO. 3: VPS35
(UniProtKB-Q96QK1 (VPS35_HUMAN); HGNC: 13487)
                10 20 30 40 50
MPTTQQSPQD EQEKLLDEAI QAVKVQSFQM KRCLDKNKLM DALKHASNML
                60 70 80 90 100
GELRTSMLSP KSYYELYMAI SDELHYLEVY LTDEFAKGRK VADLYELVQY
              110 120 130 140 150
AGNIIPRLYL LITVGVVYVK SFPQSRKDIL KDLVEMCRGV QHPLRGLFLR
              160 170 180 190 200
NYLLQCTRNI LPDEGEPTDE ETTGDISDSM DFVLLNFAEM NKLWVRMQHQ
              210 220 230 240 250
GHSRDREKRE RERQELRILV GTNLVRLSQL EGVNVERYKQ IVLTGILEQV
              260 270 280 290 300
VNCRDALAQE YLMECIIQVF PDEFHLQTLN PFLRACAELH QNVNVKNIII
              310 320 330 340 350
ALIDRLALFA HREDGPGIPA DIKLFDIFSQ QVATVIQSRQ DMPSEDVVSL
              360 370 380 390 400
QVSLINLAMK CYPDRVDYVD KVLETTVEIF NKLNLEHIAT SSAVSKELTR
              410 420 430 440 450
LLKIPVDTYN NILTVLKLKH FHPLFEYEDY ESRKSMSCYV LSNVLDYNTE
              460 470 480 490 500
IVSQDQVDSI MNLVSTLIQD QPDQPVEDPD PEDFADEQSL VGRFIHLLRS
              510 520 530 540 550
EDPDQQYLIL NTARKHFGAG GNQRIRFTLP PLVFAAYQLA FRYKENSKVD
              560 570 580 590 600
DKWEKKCQKI FSFAHQTISA LIKAELAELP LRLFLQGALA AGEIGFENHE
              610 620 630 640 650
TVAYEFMSQA FSLYEDEISD SKAQLAAITL IIGTFERMKC FSEENHEPLR
              660 670 680 690 700
TQCALAASKL LKKPDQGRAV STCAHLEWSG RNTDKNGEEL HGGKRVMECL
              710 720 730 740 750
KKALKIANQC MDPSLQVQLF IEILNRYIYF YEKENDAVTI QVLNQLIQKI
              760 770 780 790
REDLPNLESS EETEQINKHF HNTLEHLRLR RESPESEGPI YEGLIL
SEQ ID NO. 4: PRKN
(UniProtKB-060260 (PRKN_HUMAN); HGNC: 8607)
                10 20 30 40 50
MPTTQQSPQD EQEKLLDEAI QAVKVQSFQM KRCLDKNKLM DALKHASNML
                60 70 80 90 100
GELRTSMLSP KSYYELYMAI SDELHYLEVY LTDEFAKGRK VADLYELVQY
              110 120 130 140 150
AGNIIPRLYL LITVGVVYVK SFPQSRKDIL KDLVEMCRGV QHPLRGLFLR
              160 170 180 190 200
NYLLQCTRNI LPDEGEPTDE ETTGDISDSM DFVLLNFAEM NKLWVRMQHQ
              210 220 230 240 250
GHSRDREKRE RERQELRILV GTNLVRLSQL EGVNVERYKQ IVLTGILEQV
              260 270 280 290 300
VNCRDALAQE YLMECIIQVF PDEFHLQTLN PFLRACAELH QNVNVKNIII
              310 320 330 340 350
ALIDRLALFA HREDGPGIPA DIKLFDIFSQ QVATVIQSRQ DMPSEDVVSL
              360 370 380 390 400
QVSLINLAMK CYPDRVDYVD KVLETTVEIF NKLNLEHIAT SSAVSKELTR
              410 420 430 440 450
LLKIPVDTYN NILTVLKLKH FHPLFEYFDY ESRKSMSCYV LSNVLDYNTE
              460 470 480 490 500
IVSQDQVDSI MNLVSTLIQD QPDQPVEDPD PEDFADEQSL VGRFIHLLRS
              510 520 530 540 550
EDPDQQYLIL NTARKHFGAG GNQRIRFTLP PLVFAAYQLA FRYKENSKVD
              560 570 580 590 600
DKWEKKCQKI FSFAHQTISA LIKAELAELP LRLFLQGALA AGEIGFENHE
              610 620 630 640 650
TVAYEFMSQA FSLYEDEISD SKAQLAAITL IIGTFERMKC FSEENHEPLR
              660 670 680 690 700
TQCALAASKL LKKPDQGRAV STCAHLFWSG RNTDKNGEEL HGGKRVMECL
              710 720 730 740 750
KKALKIANQC MDPSLQVQLF IEILNRYIYF YEKENDAVTI QVLNQLIQKI
              760 770 780 790
REDLPNLESS EETEQINKHF HNTLEHLRLR RESPESEGPI YEGLIL
SEQ ID NO. 5: PINK1
(UniProtKB-Q9BXM7 (PINK1_HUMAN); HGNC: 14581)
                10 20 30 40 50
MAVRQALGRG LQLGRALLLR FTGKPGRAYG LGRPGPAAGC VRGERPGWAA
                60 70 80 90 100
GPGAEPRRVG LGLPNRLRFF RQSVAGLAAR LQRQFVVRAW GCAGPCGRAV
              110 120 130 140 150
FLAFGLGLGL IEEKQAESRR AVSACQEIQA IFTQKSKPGP DPLDTRRLQG
              160 170 180 190 200
FRLEEYLIGQ SIGKGCSAAV YEATMPTLPQ NLEVTKSTGL LPGRGPGTSA
              210 220 230 240 250
PGEGQERAPG APAFPLAIKM MWNISAGSSS EAILNTMSQE LVPASRVALA
              260 270 280 290 300
GEYGAVTYRK SKRGPKQLAP HPNIIRVLRA FTSSVPLLPG ALVDYPDVLP
              310 320 330 340 350
SRLHPEGLGH GRTLFLVMKN YPCTLRQYLC VNTPSPRLAA MMLLQLLEGV
              360 370 380 390 400
DHLVQQGIAH RDLKSDNILV ELDPDGCPWL VIADFGCCLA DESIGLQLPF
              410 420 430 440 450
SSWYVDRGGN GCLMAPEVST ARPGPRAVID YSKADAWAVG AIAYEIFGLV
              460 470 480 490 500
NPFYGQGKAH LESRSYQEAQ LPALPESVPP DVRQLVRALL QREASKRPSA
              510 520 530 540 550
RVAANVLHLS LWGEHILALK NLKLDKMVGW LLQQSAATLL ANRLTEKCCV
              560 570 580
ETKMKMLFLA NLECETLCQA ALLLCSWRAA L
SEQ ID NO. 6: MTHFR
(UniProtKB-P42898 (MTHR_HUMAN); HGNC: 7436)
                10 20 30 40 50
MVNEARGNSS LNPCLEGSAS SGSESSKDSS RCSTPGLDPE RHERLREKMR
                60 70 80 90 100
RRLESGDKWF SLEFFPPRTA EGAVNLISRF DRMAAGGPLY IDVTWHPAGD
              110 120 130 140 150
PGSDKETSSM MIASTAVNYC GLETILHMTC CRQRLEEITG HLHKAKQLGL
              160 170 180 190 200
KNIMALRGDP IGDQWEEEEG GFNYAVDLVK HIRSEFGDYF DICVAGYPKG
              210 220 230 240 250
HPEAGSFEAD LKHLKEKVSA GADFIITQLF FEADTFFRFV KACTDMGITC
              260 270 280 290 300
PIVPGIFPIQ GYHSLRQLVK LSKLEVPQEI KDVIEPIKDN DAAIRNYGIE
              310 320 330 340 350
LAVSLCQELL ASGLVPGLHF YTLNREMATT EVLKRLGMWT EDPRRPLPWA
              360 370 380 390 400
LSAHPKRREE DVRPIFWASR PKSYIYRTQE WDEFPNGRWG NSSSPAFGEL
              410 420 430 440 450
KDYYLFYLKS KSPKEELLKM WGEELTSEES VFEVFVLYLS GEPNRNGHKV
              460 470 480 490 500
TCLPWNDEPL AAETSLLKEE LLRVNRQGIL TINSQPNING KPSSDPIVGW
              510 520 530 540 550
GPSGGYVFQK AYLEFFTSRE TAEALLQVLK KYELRVNYHL VNVKGENITN
              560 570 580 590 600
APELQPNAVT WGIFPGREII QPTVVDPVSF MFWKDEAFAL WIERWGKLYE
              610 620 630 640 650
EESPSRTIIQ YIHDNYFLVN LVDNDFPLDN CLWQVVEDTL ELLNRPTQNA
RETEAP
```

1. A method for treatment of Parkinson's Disease (PD) in a human subject, comprising the following steps, in this order:
measuring one or more biological parameter(s) of the subject at timepoint V1;
administering nicotinamide riboside (NR) to the subject at a first dose over a first period until timepoint V2;
measuring one or more biological parameter(s) at timepoint V2, wherein:
if a biological response is achieved, the first dose is maintained, and
if a biological response is not achieved, the dose is increased to a second dose;
administering NR to the subject at the second dose over a second period until timepoint V3;
measuring one or more biological parameter(s) at timepoint V3, wherein:
if a biological response is achieved, the dose is maintained, and
if a biological response is not achieved, the dose is increased to a third dose; and
administering NR to the subject at the third dose over a third period until timepoint V4;
measuring one or more biological parameter(s) at timepoint V4, wherein:
if a biological response is achieved at V4, the dose is maintained, and
if a biological response is not achieved at V4, the treatment is discontinued;
wherein:
the biological parameter(s) is one or more selected from: cerebral NAD level(s), cerebrospinal fluid (CSF) NAD level(s), NAD-related metabolite level(s) in CSF, blood NAD level(s), and NR-related metabolic pattern (NRRP) expression; and
the biological response is defined as change in one or more of the measured biological parameter(s) relative to the respective previous timepoint and/or relative to timepoint V1.

2. The method according to claim 1, wherein one or more of the following conditions is fulfilled:
the first dose is in the range of 750 to 3000 mg daily, 1000 to 2000 mg daily, 1000 to 1500 mg daily, or 1000 mg daily;
the first period is up to 16 weeks, 2 to 12 weeks, 3-10 weeks, 8 weeks, or 4 weeks;
the second dose is in the range of 1000 to 4000 mg daily, 1000 to 3000 mg daily, 1500 to 2000 mg daily, or 2000 mg daily, provided that the second dose is higher than the first dose;
the second period is up to 16 weeks, 2 to 12 weeks, 3-10 weeks, 4-8 weeks, or 4 weeks;
the third dose is ≥1500 mg daily, 2000 to 5000 mg daily, 2000 to 3000 mg, 3000 to 4000 mg daily, or 3000 mg daily, provided that the third dose is higher than the first and second dose; and
the third period is up to 16 weeks, 2 to 12 weeks, 3-10 weeks, 4-8 weeks, or 4 weeks.

3.-4. (canceled)

5. The method according to claim 1, wherein the biological response is or comprises one or more of:
an increase of cerebral NAD levels;
an increase of blood NAD levels,
an increase of cerebral NAD levels in an occipital cortex of the subject;
an increase of cerebral NAD levels in an occipital cortex of the subject as measured by ³¹Phosphorus magnetic resonance spectroscopy (³¹P-MRS);
an increase of cerebral NAD levels in an occipital cortex of the subject as measured by determining a cerebral NAD/ATP-α molar ratio;
an increase of cerebral and/or blood NAD levels by ≥10%, ≥30%, or ≥50%;
an increase of cerebral NAD/ATP-α molar ratio by ≥10%, ≥30%, or ≥50%;
an absolute increase in the cerebral NAD/α-ATP-α molar ratio is ≥0.01, ≥0.03, ≥0.07, or ≥0.1;
an increase of CSF levels of NAD;
an increase of CSF levels of one or more metabolite(s) of the NAD-metabolome; and
an increase of a NRRP score.

6.-16. (canceled)

17. The method according to claim 1, wherein:
the first dose is 1000 mg daily;
a ³¹P-MRS scan is conducted after 30 days at timepoint V2, wherein if a cerebral NAD response is achieved at V2, treatment is continued on this maintenance dose, and if a cerebral NAD response is not achieved at V2, the dose is increased to 2000 mg;
a new 31P-MRS scan is conducted after 30 days at timepoint V3, wherein if a cerebral NAD response is achieved at V3, treatment is continued on this maintenance dose, and if a cerebral NAD response is not achieved at V3, the dose is increased to 3000 mg, or 1500 mg twice daily; and
a new 31P-MRS scan is conducted after 30 days at timepoint V4, wherein if a cerebral NAD response is achieved at that point V4, treatment is continued on this on this dose, and if a cerebral NAD response is still not achieved, the treatment is discontinued by gradual tapering of 1000 mg every week or 2000 mg daily for one week, then 1000 mg daily for one week, then stop of NR intake.

18. A method for treatment of Parkinson's Disease (PD) in a human subject in need thereof comprising administering nicotinamide riboside (NR) to the subject at a dose of more than 3000 mg daily, more than 3000 mg and up to 5000 mg daily: more than 3000 mg and up to 4000 mg daily, or 4000 to 5000 mg daily.

19. (canceled)

20. The method according to claim 18, wherein the treatment is one or more of:
safe, assessed by the absence, or substantial absence, of moderate or severe adverse events having a probable or definite causal relationship to the NR treatment; and
tolerable, assessed by the absence, or substantial absence, of mild or moderate adverse events having a definite causal relationship to the NR treatment.

21.-23. (canceled)

24. The method according to claim 18, involving one or more of:
improving clinical dysfunction in PD;
improving motor symptoms;
improving non-motor symptoms
improving cognitive symptoms;
preventing motor, non-motor and/or cognitive symptoms,
improving or preventing decay of a total MDS-UPDRS questionnaire score;
improving or preventing decay of at least one subsection I, II or III of the MDS-UPDRS questionnaire score;
improving a total MDS-UPDRS score by at least 1 or at least 2 points;
improving at least one subsection I, II or III of the MDS-UPDRS questionnaire score by at least 1 or at least 2 points;
improving or preventing decay of a NMSQ total score, assessed by the NMSQ questionnaire;
improving or preventing decay of a NMSS total score, assessed by the NMSS questionnaire;
improving or preventing decay of a MoCA total score, assessed by the MoCA questionnaire;
improving or preventing decay of a EQ-5L score, assessed by the EQ-5L questionnaire;
improving or preventing decay of a Hoehn & Yahr Stage, assessed by the Hoehn & Yahr stage in MDS-UPDRS;
altering a NAD metabolome in peripheral blood cells;
altering a NAD metabolome in CSF;
ameliorating proteostasis;
altering histone acetylation status;
change(s) in histone panacetylation level(s);
change(s) in level(s) and genomic distribution of H3K27 and H4K16 acetylation in PBMC;
decreasing neuroinflammation;
reducing level(s) of one or more inflammatory cytokine(s) in one or more of serum and CSF; and
influencing the subject's gut microbiome,
wherein said change(s) refer to one or more of: an overall change relative to placebo or absence of treatment; and a specific change concerning a given point in time or period during treatment relative to an earlier point in time or period during treatment or before the treatment or absence.

25.-27. (canceled)

28. The method according to claim 18, wherein the treatment does not involve one or more of:
altering methylation metabolism,
decreased availability of methylation substrates,
decreased availability of methyl-donors,
decreased availability of SAM,
decreased DNA methylation globally or at one or more specific site(s),
decreased synthesis of one or more neurotransmitter(s),
decreased synthesis of dopamine and serotonin,
aberrant folate metabolism, and
aberrant one-carbon metabolism,
wherein each of the above refers to one or more of: an overall change relative to placebo or absence; and a specific change concerning a given point in time or period during treatment relative to an earlier point in time or period during treatment or before the treatment or absence.

29.-40. (canceled)

41. The method according to claim 18, wherein the treatment involves or acts as one or more of preventing progression of PD; decreasing progression of PD; delaying progression of PD; a neuroprotective therapy; a neuroprotective, disease modifying therapy for PD dementia (PDD); a neuroprotective, disease modifying therapy for dementia with Lewy bodies (DLB); a neuroprotective, disease modifying therapy for PDD and DLB; and delaying nigrostriatal degeneration or denervation.

42. (canceled)

43. The method according to claim 18, wherein the subject fulfils one or more of:
is newly diagnosed with PD;
has been diagnosed with PD within 1, 2, 3, 4 or 5 years before beginning of treatment;
the subject's first PD symptom has been observed 50 months or less, 45 months or less, 40 months or less, 30 months or less, 35 months or less, 25 months or less, 20 months or less, 15 months or less, 10 months or less, or 5 months or less before beginning of treatment;
has nigrostriatal degeneration or denervation at beginning of treatment;
is drug naïve with respect to dopaminergic treatment prior to the treatment;
has an age of ≥35 years, ≥40 years, ≥50 years, ≥60 years, ≥65 years, 35 to 85, 40 to 80, 50 to 80, 60 to 75, or 65 to 75 years at beginning of treatment;
has clinical diagnosis of idiopathic PD at beginning of treatment;
has a Hoehn & Yahr score <4, optionally <3, at beginning of treatment;
has an age of ≥40 years at beginning of treatment;
has no dementia or other neurodegenerative disorder at beginning of treatment;
has not been diagnosed with atypical parkinsonism, in particular PSP, MSA, CBD, or vascular parkinsonism at beginning of treatment;
has no metabolic, neoplastic, or other physically or mentally debilitating disorder at beginning of treatment; and
has not used high-dose vitamin B3 supplementation, such as 500 mg or more of niacin daily, within 30 days before beginning of treatment.

44.-49. (canceled)

50. The method according to claim 18, wherein the PD is selected from early PD, idiopathic (IDP), juvenile; early-onset parkinsonism; secondary parkinsonism; atypical parkinsonism; vascular parkinsonism; drug-induced parkinsonism; multiple system atrophy (MSA); progressive supranuclear palsy; and monogenic PD.

51.-58. (canceled)

59. The method according to claim 18, involving oral administration of NR to the subject.

60. The method according to claim 18, wherein NR is administered as a monotherapy, or in combination with a dopaminergic agent plus MAO-B inhibitor.

61.-62. (canceled)

63. The method according to claim 60, wherein:
(a) 8-12 mg selegiline per day, 150-800 mg or 300-450 mg levodopa per day, and 37.5-200 mg or 75-112.5 mg carbidopa per day are administered to the subject;
(b) 10 mg selegiline once per day, 100 mg levodopa three times per day and 25 mg carbidopa three times per day are administered to the subject;
(c) 8-12 mg selegiline per day, 150-800 mg or 300-450 mg levodopa per day, and 37.5-200 mg or 75-112.5 mg benserazide per day are administered to the subject; or
(d) 10 mg once per day, 100 mg levodopa three times per day and 25 mg benserazide three times per day are administered to the subject.

64.-66. (canceled)

67. The method according to claim 18, wherein the overall treatment duration is at least 2, at least 3, at least 6, or at least 12 months.

68. (canceled)

69. The method according to claim 18, wherein NR is administered:
as a pharmaceutically acceptable salt, solvate and/or hydrate thereof; or
as a salt, solvate and/or hydrate thereof, the salt being selected from one or more of fluoride, chloride, bromide, iodide, formate, acetate, ascorbate, aspartate, benzoate, butyrate, carbonate, citrate, carbamate, formate, gluconate, glutamate, lactate, malate, methyl bromide, methyl sulfate, nitrate, phosphate, propionate, diphosphate, succinate, sulfate, sulfonate, hydrogen tartrate, hydrogen malate, trifluoroacetate, tribromomethanesulfonate, trichloromethanesulfonate, and trifluoromethanesulfonate.

70.-84. (canceled)

85. The method according to claim 18, wherein NR is administered as the sole active ingredient, or as the sole active ingredient in combination with one or more of a dopaminergic agent and MAO-B inhibitor.

86. A dosage form comprising nicotinamide riboside (NR) as a pharmaceutically acceptable salt, solvate and/or hydrate thereof; or as a salt, solvate and/or hydrate thereof, the salt being selected from one or more of fluoride, chloride, bromide, iodide, formate, acetate, ascorbate, aspartate, benzoate, butyrate, carbonate, citrate, carbamate, formate, gluconate, glutamate, lactate, malate, methyl bromide, methyl sulfate, nitrate, phosphate, propionate, diphosphate, succinate, sulfate, sulfonate, hydrogen tartrate, hydrogen malate, trifluoroacetate, tribromomethanesulfonate, trichloromethanesulfonate, and trifluoromethanesulfonate,
the dosage form comprising NR in an amount of >2000 mg, ≥2500 mg, ≥3000 mg, ≥4000 mg, >2000 mg and ≤5000 mg, 2500-5000 mg, 3000-4000 mg, 4000-5000 mg, or 3000 mg per dosage unit, and optionally one or more pharmaceutically acceptable excipient(s).

87.-96. (canceled)

97. The dosage form according to claim 86, further comprising one or more of: microcrystalline cellulose, hydroxypropyl methylcellulose, hypromellose, calcium laurate, magnesium stearate, silicon dioxide, one or more methyl donor(s), and betaine (trimethylglycine).

98.-99. (canceled)

100. The dosage form according to claim 86, which is an oral capsule comprising:
a) nicotinamide riboside chloride, microcrystalline cellulose, hydroxypropyl methylcellulose and magnesium stearate; or
b) nicotinamide riboside chloride, hypromellose, leucine, microcrystalline cellulose, and silicon dioxide; or
c) nicotinamide riboside hydrogen malate, microcrystalline cellulose, hydroxypropyl methylcellulose, calcium laurate, silicium dioxide, one or more methyl donor(s), and betaine (trimethylglycine).


SEQUENCE LISTING (11 sequences)

SEQ ID NO: 1 - protein, 140 aa, Homo sapiens
  REGION 1..140: note = SNCA (UniprotKB - P37840 (SYUA_HUMAN); HGNC: 11138, SNCA)
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - protein, 2527 aa, Homo sapiens
  REGION 1..2527: note = LRRK2 (UniProtKB - Q5S007 (LRRK2_HUMAN); HGNC: 18618)
MASGSCQGCEEDEETLKKLIVRLNNVQEGKQIETLVQILEDLLVFTYSERASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 3 - protein, 796 aa, Homo sapiens
  REGION 1..796: note = VPS35 (UniProtKB - Q96QK1 (VPS35_HUMAN); HGNC:13487)
MPTTQQSPQDEQEKLLDEAIQAVKVQSFQMKRCLDKNKLMDALKHASNMLGELRTSMLSPKSYYELYMAISDELHYLEVYLTDEFAKGRKVADLYELVQYAGNIIPRLYLLITVGVVYVKSFPQSRKDILKDLVEMCRGVQHPLRGLFLRNYLLQCTRNILPDEGEPTDEETTGDISDSMDFVLLNFAEMNKLWVRMQHQGHSRDREKRERERQELRILVGTNLVRLSQLEGVNVERYKQIVLTGILEQVVNCRDALAQEYLMECIIQVFPDEFHLQTLNPFLRACAELHQNVNVKNIIIALIDRLALFAHREDGPGIPADIKLFDIFSQQVATVIQSRQDMPSEDVVSLQVSLINLAMKCYPDRVDYVDKVLETTVEIFNKLNLEHIATSSAVSKELTRLLKIPVDTYNNILTVLKLKHFHPLFEYFDYESRKSMSCYVLSNVLDYNTEIVSQDQVDSIMNLVSTLIQDQPDQPVEDPDPEDFADEQSLVGRFIHLLRSEDPDQQYLILNTARKHFGAGGNQRIRFTLPPLVFAAYQLAFRYKENSKVDDKWEKKCQKIFSFAHQTISALIKAELAELPLRLFLQGALAAGEIGFENHETVAYEFMSQAFSLYEDEISDSKAQLAAITLIIGTFERMKCFSEENHEPLRTQCALAASKLLKKPDQGRAVSTCAHLFWSGRNTDKNGEELHGGKRVMECLKKALKIANQCMDPSLQVQLFIEILNRYIYFYEKENDAVTIQVLNQLIQKIREDLPNLESSEETEQINKHFHNTLEHLRLRRESPESEGPIYEGLIL

SEQ ID NO: 4 - protein, 465 aa, Homo sapiens
  REGION 1..465: note = PRKN (UniProtKB - O60260 (PRKN_HUMAN); HGNC:8607)
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 5 - protein, 581 aa, Homo sapiens
  REGION 1..581: note = PINK1 (UniProtKB - Q9BXM7 (PINK1_HUMAN); HGNC:14581)
MAVRQALGRGLQLGRALLLRFTGKPGRAYGLGRPGPAAGCVRGERPGWAAGPGAEPRRVGLGLPNRLRFFRQSVAGLAARLQRQFVVRAWGCAGPCGRAVFLAFGLGLGLIEEKQAESRRAVSACQEIQAIFTQKSKPGPDPLDTRRLQGFRLEEYLIGQSIGKGCSAAVYEATMPTLPQNLEVTKSTGLLPGRGPGTSAPGEGQERAPGAPAFPLAIKMMWNISAGSSSEAILNTMSQELVPASRVALAGEYGAVTYRKSKRGPKQLAPHPNIIRVLRAFTSSVPLLPGALVDYPDVLPSRLHPEGLGHGRTLFLVMKNYPCTLRQYLCVNTPSPRLAAMMLLQLLEGVDHLVQQGIAHRDLKSDNILVELDPDGCPWLVIADFGCCLADESIGLQLPFSSWYVDRGGNGCLMAPEVSTARPGPRAVIDYSKADAWAVGAIAYEIFGLVNPFYGQGKAHLESRSYQEAQLPALPESVPPDVRQLVRALLQREASKRPSARVAANVLHLSLWGEHILALKNLKLDKMVGWLLQQSAATLLANRLTEKCCVETKMKMLFLANLECETLCQAALLLCSWRAAL

SEQ ID NO: 6 - protein, 656 aa, Homo sapiens [sequence omitted]

SEQ ID NO: 7 - DNA, 3177 bp, Homo sapiens
  misc_feature 1..3177: note = SNCA (cDNA - NCBI Reference Sequence: NM_000345.4)
  source 1..3177: mol_type = genomic DNA
ggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 8 - DNA, 9239 bp, Homo sapiens
  misc_feature 1..9239: note = LRRK2 (cDNA - NCBI Reference Sequence: NM_198578.4)
  source 1..9239: mol_type = genomic DNA
ggggcccgcggggagcgctggctgcgggcggtgagctgagctcgcccccggggagctgtggccggcgcccctgccggttccctgagcagcggacgttcatgctgggagggcggcgggttggaagcaggtgccaccatggctagtggcagctgtcaggggtgcgaagaggacgaggaaactctgaagaagttgatagtcaggctgaacaatgtccaggaaggaaaacagatagaaacgctggtccaaatcctggaggatctgctggtgttcacgtactccgagcgcgcctccaagttatttcaaggcaaaaatatccatgtgcctctgttgatcgtcttggactcctatatgagagtcgcgagtgtgcagcaggtgggttggtcacttctgtgcaaattaatagaagtctgtccaggtacaatgcaaagcttaatgggaccccaggatgttggaaatgattgggaagtccttggtgttcaccaattgattcttaaaatgctaacagttcataatgccagtgtaaacttgtcagtgattggactgaagaccttagatctcctcctaacttcaggtaaaatcaccttgctgatattggatgaagaaagtgatattttcatgttaatttttgatgccatgcactcatttccagccaatgatgaagtccagaaacttggatgcaaagctttacatgtgctgtttgagagagtctcagaggagcaactgactgaatttgttgagaacaaagattatatgatattgttaagtgcgttaacaaattttaaagatgaagaggaaattgtgcttcatgtgctgcattgtttacattccctagcgattccttgcaataatgtggaagtcctcatgagtggcaatgtcaggtgttataatattgtggtggaagctatgaaagcattccctatgagtgaaagaattcaagaagtgagttgctgtttgctccataggcttacattaggtaattttttcaatatcctggtattaaacgaagtccatgagtttgtggtgaaagctgtgcagcagtacccagagaatgcagcattgcagatctcagcgctcagctgtttggccctcctcactgagactattttcttaaatcaagatttagaggaaaagaatgagaatcaagagaatgatgatgagggggaagaagataaattgttttggctggaagcctgttacaaagcattaacgtggcatagaaagaacaagcacgtgcaggaggccgcatgctgggcactaaataatctccttatgtaccaaaacagtttacatgagaagattggagatgaagatggccatttcccagctcatagggaagtgatgctctccatgctgatgcattcttcatcaaaggaagttttccaggcatctgcgaatgcattgtcaactctcttagaacaaaatgttaatttcagaaaaatactgttatcaaaaggaatacacctgaatgttttggagttaatgcagaagcatatacattctcctgaagtggctgaaagtggctgtaaaatgctaaatcatctttttgaaggaagcaacacttccctggatataatggcagcagtggtccccaaaatactaacagttatgaaacgtcatgagacatcattaccagtgcagctggaggcgcttcgagctattttacattttatagtgcctggcatgccagaagaatccagggaggatacagaatttcatcataagctaaatatggttaaaaaacagtgtttcaagaatgatattcacaaactggtcctagcagctttgaacaggttcattggaaatcctgggattcagaaatgtggattaaaagtaatttcttctattgtacattttcctgatgcattagagatgttatccctggaaggtgctatggattcagtgcttcacacactgcagatgtatccagatgaccaagaaattcagtgtctgggtttaagtcttataggatacttgattacaaagaagaatgtgttcataggaactggacatctgctggcaaaaattctggtttccagcttataccgatttaaggatgttgctgaaatacagactaaaggatttcagacaatcttagcaatcctcaaattgtcagcatctttttctaagctgctggtgcatcattcatttgacttagtaatattccatcaaatgtcttccaatatcatggaacaaaaggatcaacagtttctaaacctctgttgcaagtgttttgcaaaagtagctatggatgattacttaaaaaatgtgatgctagagagagcgtgtgatcagaataacagcatcatggttgaatgcttgcttctattgggagcagatgccaatcaagcaaaggagggatcttctttaatttgtcaggtatgtgagaaagagagcagtcccaaattggtggaactcttactgaatagtggatctcgtgaacaagatgtacgaaaagcgttgacgataagcattgggaaaggtgacagccagatcatcagcttgctcttaaggaggctggccctggatgtggccaacaatagcatttgccttggaggattttgtataggaaaagttgaaccttcttggcttggtcctttatttccagataagacttctaatttaaggaaacaaacaaatatagcatctacactagcaagaatggtgatcagatatcagatgaaaagtgctgtggaagaaggaacagcctcaggcagcgatggaaatttttctgaagatgtgctgtctaaatttgatgaatggacctttattcctgactcttctatggacagtgtgtttgctcaaagtgatgacctggatagtgaaggaagtgaaggctcatttcttgtgaaaaagaaatctaattcaattagtgtaggagaattttaccgagatgccgtattacagcgttgctcaccaaatttgcaaagacattccaattccttggggcccatttttgatcatgaagatttactgaagcgaaaaagaaaaatattatcttcagatgattcactcaggtcatcaaaacttcaatcccatatgaggcattcagacagcatttcttctctggcttctgagagagaatatattacatcactagacctttcagcaaatgaactaagagatattgatgccctaagccagaaatgctgtataagtgttcatttggagcatcttgaaaagctggagcttcaccagaatgcactcacgagctttccacaacagctatgtgaaactctgaagagtttgacacatttggacttgcacagtaataaatttacatcatttccttcttatttgttgaaaatgagttgtattgctaatcttgatgtctctcgaaatgacattggaccctcagtggttttagatcctacagtgaaatgtccaactctgaaacagtttaacctgtcatataaccagctgtcttttgtacctgagaacctcactgatgtggtagagaaactggagcagctcattttagaaggaaataaaatatcagggatatgctcccccttgagactgaaggaactgaagattttaaaccttagtaagaaccacatttcatccctatcagagaactttcttgaggcttgtcctaaagtggagagtttcagtgccagaatgaattttcttgctgctatgcctttcttgcctccttctatgacaatcctaaaattatctcagaacaaattttcctgtattccagaagcaattttaaatcttccacacttgcggtctttagatatgagcagcaatgatattcagtacctaccaggtcccgcacactggaaatctttgaacttaagggaactcttatttagccataatcagatcagcatcttggacttgagtgaaaaagcatatttatggtctagagtagagaaactgcatctttctcacaataaactgaaagagattcctcctgagattggctgtcttgaaaatctgacatctctggatgtcagttacaacttggaactaagatcctttcccaatgaaatggggaaattaagcaaaatatgggatcttcctttggatgaactgcatcttaactttgattttaaacatataggatgtaaagccaaagacatcataaggtttcttcaacagcgattaaaaaaggctgtgccttataaccgaatgaaacttatgattgtgggaaatactgggagtggtaaaaccaccttattgcagcaattaatgaaaaccaagaaatcagatcttggaatgcaaagtgccacagttggcatagatgtgaaagactggcctatccaaataagagacaaaagaaagagagatctcgtcctaaatgtgtgggattttgcaggtcgtgaggaattctatagtactcatccccattttatgacgcagcgagcattgtaccttgctgtctatgacctcagcaagggacaggctgaagttgatgccatgaagccttggctcttcaatataaaggctcgcgcttcttcttcccctgtgattctcgttggcacacatttggatgtttctgatgagaagcaacgcaaagcctgcatgagtaaaatcaccaaggaactcctgaataagcgagggttccctgccatacgagattaccactttgtgaatgccaccgaggaatctgatgctttggcaaaacttcggaaaaccatcataaacgagagccttaatttcaagatccgagatcagcttgttgttggacagctgattccagactgctatgtagaacttgaaaaaatcattttatcggagcgtaaaaatgtgccaattgaatttcccgtaattgaccggaaacgattattacaactagtgagagaaaatcagctgcagttagatgaaaatgagcttcctcacgcagttcactttctaaatgaatcaggagtccttcttcattttcaagacccagcactgcagttaagtgacttgtactttgtggaacccaagtggctttgtaaaatcatggcacagattttgacagtgaaagtggaaggttgtccaaaacaccctaagggcattatttcgcgtagagatgtggaaaaatttctttcaaaaaaaaggaaatttccaaagaactacatgtcacagtattttaagctcctagaaaaattccagattgctttgccaataggagaagaatatttgctggttccaagcagtttgtctgaccacaggcctgtgatagagcttccccattgtgagaactctgaaattatcatccgactatatgaaatgccttattttccaatgggattttggtcaagattaatcaatcgattacttgagatttcaccttacatgctttcagggagagaacgagcacttcgcccaaacagaatgtattggcgacaaggcatttacttaaattggtctcctgaagcttattgtctggtaggatctgaagtcttagacaatcatccagagagtttcttaaaaattacagttccttcttgtagaaaaggctgtattcttttgggccaagttgtggaccacattgattctctcatggaagaatggtttcctgggttgctggagattgatatttgtggtgaaggagaaactctgttgaagaaatgggcattatatagttttaatgatggtgaagaacatcaaaaaatcttacttgatgacttgatgaagaaagcagaggaaggagatctcttagtaaatccagatcaaccaaggctcaccattccaatatctcagattgcccctgacttgattttggctgacctgcctagaaatattatgttgaataatgatgagttggaatttgaacaagctccagagtttctcctaggtgatggcagttttggatcagtttaccgagcagcctatgaaggagaagaagtggctgtgaagatttttaataaacatacatcactcaggctgttaagacaagagcttgtggtgctttgccacctccaccaccccagtttgatatctttgctggcagctgggattcgtccccggatgttggtgatggagttagcctccaagggttccttggatcgcctgcttcagcaggacaaagccagcctcactagaaccctacagcacaggattgcactccacgtagctgatggtttgagatacctccactcagccatgattatataccgagacctgaaaccccacaatgtgctgcttttcacactgtatcccaatgctgccatcattgcaaagattgctgactacggcattgctcagtactgctgtagaatggggataaaaacatcagagggcacaccagggtttcgtgcacctgaagttgccagaggaaatgtcatttataaccaacaggctgatgtttattcatttggtttactactctatgacattttgacaactggaggtagaatagtagagggtttgaagtttccaaatgagtttgatgaattagaaatacaaggaaaattacctgatccagttaaagaatatggttgtgccccatggcctatggttgagaaattaattaaacagtgtttgaaagaaaatcctcaagaaaggcctacttctgcccaggtctttgacattttgaattcagctgaattagtctgtctgacgagacgcattttattacctaaaaacgtaattgttgaatgcatggttgctacacatcacaacagcaggaatgcaagcatttggctgggctgtgggcacaccgacagaggacagctctcatttcttgacttaaatactgaaggatacacttctgaggaagttgctgatagtagaatattgtgcttagccttggtgcatcttcctgttgaaaaggaaagctggattgtgtctgggacacagtctggtactctcctggtcatcaataccgaagatgggaaaaagagacataccctagaaaagatgactgattctgtcacttgtttgtattgcaattccttttccaagcaaagcaaacaaaaaaattttcttttggttggaaccgctgatggcaagttagcaatttttgaagataagactgttaagcttaaaggagctgctcctttgaagatactaaatataggaaatgtcagtactccattgatgtgtttgagtgaatccacaaattcaacggaaagaaatgtaatgtggggaggatgtggcacaaagattttctccttttctaatgatttcaccattcagaaactcattgagacaagaacaagccaactgttttcttatgcagctttcagtgattccaacatcataacagtggtggtagacactgctctctatattgctaagcaaaatagccctgttgtggaagtgtgggataagaaaactgaaaaactctgtggactaatagactgcgtgcactttttaagggaggtaatggtaaaagaaaacaaggaatcaaaacacaaaatgtcttattctgggagagtgaaaaccctctgccttcagaagaacactgctctttggataggaactggaggaggccatattttactcctggatctttcaactcgtcgacttatacgtgtaatttacaacttttgtaattcggtcagagtcatgatgacagcacagctaggaagccttaaaaatgtcatgctggtattgggctacaaccggaaaaatactgaaggtacacaaaagcagaaagagatacaatcttgcttgaccgtttgggacatcaatcttccacatgaagtgcaaaatttagaaaaacacattgaagtgagaaaagaattagctgaaaaaatgagacgaacatctgttgagtaagagagaaataggaattgtctttggataggaaaattattctctcctcttgtaaatatttattttaaaaatgttcacatggaaagggtactcacattttttgaaatagctcgtgtgtatgaaggaatgttattatttttaatttaaatatatgtaaaaatacttaccagtaaatgtgtattttaaagaactatttaaaacacaatgttatatttcttataaataccagttactttcgttcattaattaatgaaaataaatctgtgaagtacctaatttaagtactcatactaaaatttataaggccgataattttttgttttcttgtctgtaatggaggtaaactttattttaaattctgtgcttaagacaggactattgcttgtcgatttttctagaaatctgcacggtataatgaaaatattaagacagtttcccatgtaatgtattccttcttagattgcatcgaaatgcactatcatatatgcttgtaaatattcaaatgaatttgcactaataaagtcctttgttggtatgtgaattctctttgttgctgttgcaaacagtgcatcttacacaacttcactcaattcaaaagaaaactccattaaaagtactaatgaaaaaacatgacatactgtcaaagtcctcatatctaggaaagacacagaaactctctttgtcacagaaactctctgtgtctttcctagacataatagagttgtttttcaactctatgtttgaatgtggataccctgaattttgtataattagtgtaaatacagtgttcagtccttcaagtgatatttttatttttttattcataccactagctacttgttttctaatctgcttcattctaatgcttatattcatcttttccctaaatttgtgatgctgcagatcctacatcattcagatagaaacctttttttttttcagaattatagaattccacagctcctaccaagaccatgaggataaatatctaacacttttcagttgctgaaggagaaaggagctttagttatgatggataaaaatatctgccaccctaggcttccaaattatacttaaattgtttacatagcttaccacaataggagtatcagggccaaatacctatgtaataatttgaggtcatttctgctttaggaaaagtactttcggtaaattctttggccctgaccagtattcattatttcagataattccctgtgataggacaactagtacatttaatattctcagaacttatggcattttactatgtgaaaactttaaatttatttatattaagggtaatcaaattcttaaagatgaaagattttctgtattttaaaggaagctatgctttaacttgttatgtaattaacaaaaaaatcatatataatagagctctttgttccagtgttatctctttcattgttactttgtatttgcaattttttttaccaaagacaaattaaaaaaatgaataccatatttaaatggaataataaaggttttttaaaaactttaaa

SEQ ID NO: 9 - DNA, 6776 bp, Homo sapiens
  misc_feature 1..6776: note = VPS35 (cDNA - NCBI Reference Sequence: NM_018206.6)
  source 1..6776: mol_type = genomic DNA
acgcgcggggcgggtgctgcttgctgcaggctctggggagtcgccatgcctacaacacagcagtcccctcaggatgagcaggaaaagctcttggatgaagccatacaggctgtgaaggtccagtcattccaaatgaagagatgcctggacaaaaacaagcttatggatgctctaaaacatgcttctaatatgcttggtgaactccggacttctatgttatcaccaaagagttactatgaactttatatggccatttctgatgaactgcactacttggaggtctacctgacagatgagtttgctaaaggaaggaaagtggcagatctctacgaacttgtacagtatgctggaaacattatcccaaggctttaccttttgatcacagttggagttgtatatgtcaagtcatttcctcagtccaggaaggatattttgaaagatttggtagaaatgtgccgtggtgtgcaacatcccttgaggggtctgtttcttcgaaattaccttcttcagtgtaccagaaatatcttacctgatgaaggagagccaacagatgaagaaacaactggtgacatcagtgattccatggattttgtactgctcaactttgcagaaatgaacaagctctgggtgcgaatgcagcatcagggacatagccgagatagagaaaaaagagaacgagaaagacaagaactgagaattttagtgggaacaaatttggtgcgcctcagtcagttggaaggtgtaaatgtggaacgttacaaacagattgttttgactggcatattggagcaagttgtaaactgtagggatgctttggctcaagaatatctcatggagtgtattattcaggttttccctgatgaatttcacctccagactttgaatccttttcttcgggcctgtgctgagttacaccagaatgtaaatgtgaagaacataatcattgctttaattgatagattagctttatttgctcaccgtgaagatggacctggaatcccagcggatattaaactttttgatatattttcacagcaggtggctacagtgatacagtctagacaagacatgccttcagaggatgttgtatctttacaagtctctctgattaatcttgccatgaaatgttaccctgatcgtgtggactatgttgataaagttctagaaacaacagtggagatattcaataagctcaaccttgaacatattgctaccagtagtgcagtttcaaaggaactcaccagacttttgaaaataccagttgacacttacaacaatattttaacagtcttgaaattaaaacattttcacccactctttgagtactttgactacgagtccagaaagagcatgagttgttatgtgcttagtaatgttctggattataacacagaaattgtctctcaagaccaggtggattccataatgaatttggtatccacgttgattcaagatcagccagatcaacctgtagaagaccctgatccagaagattttgctgatgagcagagccttgtgggccgcttcattcatctgctgcgctctgaggaccctgaccagcagtacttgattttgaacacagcacgaaaacattttggagctggtggaaatcagcggattcgcttcacactgccacctttggtatttgcagcttaccagctggcttttcgatataaagagaattctaaagtggatgacaaatgggaaaagaaatgccagaagattttttcatttgcccaccagactatcagtgctttgatcaaagcagagctggcagaattgcccttaagactttttcttcaaggagcactagctgctggggaaattggttttgaaaatcatgagacagtcgcatatgaattcatgtcccaggcattttctctgtatgaagatgaaatcagcgattccaaagcacagctagctgccatcaccttgatcattggcacttttgaaaggatgaagtgcttcagtgaagagaatcacgaacctctgaggactcagtgtgcccttgctgcatccaaacttctaaagaaacctgatcagggccgagctgtgagcacctgtgcacatctcttctggtctggcagaaacacggacaaaaatggggaggagcttcacggaggcaagagggtaatggagtgcctaaaaaaagctctaaaaatagcaaatcagtgcatggacccctctctacaagtgcagctttttatagaaattctgaacagatatatctatttttatgaaaaggaaaatgatgcggtaacaattcaggttttaaaccagcttatccaaaagattcgagaagacctcccgaatcttgaatccagtgaagaaacagagcagattaacaaacattttcataacacactggagcatttgcgcttgcggcgggaatcaccagaatccgaggggccaatttatgaaggtctcatcctttaaaaaggaaatagctcaccatactcctttccatgtacatccagtgagggttttattacgctaggtttcccttccatagattgtgcctttcagaaatgctgaggtaggtttcccatttcttacctgtgatgtgttttacccagcacctccggacactcaccttcaggaccttaataaaattattcacttggtaagtgttcaagtctttctgatcaccccaagtagcatgactgatctgcaatttaaaattcctgtgatctgtaaaaaaaaaaaaaaaaaaaaacacaaaacccacaagcacttatcttggctactaatgaagctctccttttttttgtttgtttgtttgcttcattgttgattgtgtattttcttcattcctggggagtactaacccaaaagcgtctgtctcttgttttctagtccagtttgagattaatttagaagaaaggaatactgtatgtgaaattcatcttgggctttcccctaaattgcaagataaggccatgtgtaagattttccctaaaactagaatatattaatgcatgtttgagaattttaaagcaccatggtcaaaaccagaagctatattttgcatatttggactcagccatccattaagaacccatgttgtcctctggacatatttatcaatataattgggttttaaatagtataaaagaaaacttgtgatctatataatttatgtatcaccttcattgtaaatttagcaggaaatgcatcacaattatgatttttttttttgcaccagtgaaacaataaagatgctattaacaattttggagctttttttcccctttcatactatctttaattgataaaaagtactagtagtatggctatagagtttaatagtcattctcatatattcacagagctcacttaactctagttttaagcacttgttgaacaaggcagcatctactcttttagtcatttataccccttttctttcctcaactttaccctgcttttcccaaggcatgatgaattcacataaaccccccaaaggctcaccaccgatactagttacctactatataataaaccatcccaaaacttactgtcttaaaacagcaaacatttatctcacaggtctctggatcaggaatttgggagcagcttagctggtggttctggcaaagagtgtggtgtaaggttgcaaacaagatactggttggggctgcagtcatctgatgctttgactggggctgacttttctgctcactgacctggctgttaggcagaggccccagttcctcttcttgagggcagaggccccagttcctctccttgagggcagaggcctaagttcctttccttgagggcagaggccccagttcctctccttgacggcccttccatagagtgtcctcacagcatggtggctggctttcccctgagtatgaaagcaagaaggaaaccacagtgcctttcagtggcctccctgtggaagtgacatgctcatttttgccttattctgtaagtggggagtcactaagtctagcctatattcaagggtaaggagaattaagctccacctcttaaagggaaaatttatagacattttcaaatgactacatcacttaacccctcaccatctgccctcccattgctagcacttgatgactagcccttgctgggctttacatgaacagatgtttcccaaagttataaaattagtaccactaaaatgtatcaaatgttaagccattctgtggtatgtcatagtccagcaaaccctttctgttctttgtaggtgttagtttaccccgtataaacctgattatggcttgacatgtccatattatttacatgttttgtgaaatacaactggaatagtagaaaagacatgagtcaagaacacttgagttggagagcttggtttgcaagtaaggtaatgtcagtacagtgtgtaaattatctcttattttctgggttgaggtaaacccaattttaaaaataataaaccaacaaaagtaaataaaagtgaaacaaaagcactgctagtatccttaacttacagattttttttgttgaacaaaaactgggtaccagatatagttaggcactgaatctaaaggtgaagaagatcatagcctctagcttcaagaacgttagtctgctgtgaagacagacctgtagttacagaagatggtaagggtttgtgcaggagagcagtgaagaagagctttgagggattggggatggggaagttctgcagagggttgtatgaattctcatggaacatcaagtccaatagtttatttagccttctgaggccagtgctttggcctgcagtcccaggctcctcagacccatgggctgaagcaagttatactcttggctgcctgtagtgaaatagatatttagaaattgacttagcatttaaaaccctttatagcaatttgacagagtaattttatgtctattggatgtaataaatatttggctttgtgctttgtgtgtctgctattccctgttctccttttttagtaatttttattgtgtcttacaaaaaagtacctgtccttaattggggtgtggtggggaatccctcacaggtggatggaaacgctgaaccagggcaggcttggtgctgaatggcattaaattccaagactaggggatagtttggggggacacatctaggaagatgctgtgactggatgagagatgcctgccgtgttagactttcctaccctccctttcctgcctctacccaggaaaccagtgattcttagagtccagatgatcccttctttgcctagaaattcatgtttctgactcatcctcaagcctgaaattggtggtcatcaggttaatgaactgcaaaaacagcccctgctgatagacactgctcaaagagagaggcgtttgagttgatcaggcaccagagcacctagaaggagaatgatgagcacttgtgtacagttcgtgtgcagctctcaaaggcattgagaaatgtgacagacctcaaggaataaatgcaccaccacaggcactgaggggatttaaacacaaagtctaggttccagaggggtcttctgttaacatttgctctctcattcagagacctcaaggtaataagctcaagcagaaaggatttcccaacagaattgtctttgacaatgggaattccagactgcatattgtaaatgaaaggtagagaaatctgtgcaggagaccttcctgtttgctggggtttgtcctatggggatgctgtaggcatggacaacagacactgattcaggtgccagtatctacacatttaccatacacttgaattcccaagagtagtgacttgccttaggtttgattaaagacaaatgtttcctggcttgtggtcttgtattgctgattactccagtagggcaccctgcattgtccaacaaaatggtccatgcttccaaaatctctggagagggactgtatattttatttttttgagacggagtcttgctctgtcacccaagcaggagtgcagtggtgcgatcttggctcactgcaacctccgcctccccagttcaagcaattctcctgcctcagcctcccgagtagctgggattacaggcgtgcaccaccatgcctggctaatttttgtatttttagtacagatgggggtttcaccatgttggccaggctggtcacgaacttctgaccttgtgatccacctggctcagcctcccaaagtgctggaattacaggcatgagccaccttgcccagccagattgtatattgtaccactcattgctgtgttcaggcttgcatcagtagtgtcttcaaataagtaaaatcttaggctgtgacttaaatccattttcacttagagaaatagaaacacaaggaaacctttggacgcttcataactgctgggaaaggggtattatcaattgtgtttgagagtcaaactataaattacttcccaaggttagttctacctatgcccaggaatgaacaaggacagcttaaaggttagaagcaagatggagtcatttgggtctgatctctttcactgtcataatttcctcagttacaatttttgtaaaggtggtttcaaatgctttgctgacctcccataaacaaggatgtgccaattgtaacttcagttctgcaattcaagtctgctcccaaaactaaagtccattgaattgcatactgattgataatgttcattacgagtttatcttcccatgtgcagagcaaagacaagaggagatcagccattcctccacctacccagagacgttgcattggaaaggagccgtatcttctagtatggttttgcaaatgtaaaatgtaaaatctatgcacgctcaca

SEQ ID NO: 10 - DNA, 2657 bp, Homo sapiens
  misc_feature 1..2657: note = PINK1 (cDNA - NCBI Reference Sequence: NM_032409.3)
  source 1..2657: mol_type = genomic DNA
agaggcaccgccccaagtttgttgtgaccggcgggggacgccggtggtggcggcagcggcggctgcgggggcaccgggccgcggcgccaccatggcggtgcgacaggcgctgggccgcggcctgcagctgggtcgagcgctgctgctgcgcttcacgggcaagcccggccgggcctacggcttggggcggccgggcccggcggcgggctgtgtccgcggggagcgtccaggctgggccgcaggaccgggcgcggagcctcgcagggtcgggctcgggctccctaaccgtctccgcttcttccgccagtcggtggccgggctggcggcgcggttgcagcggcagttcgtggtgcgggcctggggctgcgcgggcccttgcggccgggcagtctttctggccttcgggctagggctgggcctcatcgaggaaaaacaggcggagagccggcgggcggtctcggcctgtcaggagatccaggcaatttttacccagaaaagcaagccggggcctgacccgttggacacgagacgcttgcagggctttcggctggaggagtatctgatagggcagtccattggtaagggctgcagtgctgctgtgtatgaagccaccatgcctacattgccccagaacctggaggtgacaaagagcaccgggttgcttccagggagaggcccaggtaccagtgcaccaggagaagggcaggagcgagctccgggggcccctgccttccccttggccatcaagatgatgtggaacatctcggcaggttcctccagcgaagccatcttgaacacaatgagccaggagctggtcccagcgagccgagtggccttggctggggagtatggagcagtcacttacagaaaatccaagagaggtcccaagcaactagcccctcaccccaacatcatccgggttctccgcgccttcacctcttccgtgccgctgctgccaggggccctggtcgactaccctgatgtgctgccctcacgcctccaccctgaaggcctgggccatggccggacgctgttcctcgttatgaagaactatccctgtaccctgcgccagtacctttgtgtgaacacacccagcccccgcctcgccgccatgatgctgctgcagctgctggaaggcgtggaccatctggttcaacagggcatcgcgcacagagacctgaaatccgacaacatccttgtggagctggacccagacggctgcccctggctggtgatcgcagattttggctgctgcctggctgatgagagcatcggcctgcagttgcccttcagcagctggtacgtggatcggggcggaaacggctgtctgatggccccagaggtgtccacggcccgtcctggccccagggcagtgattgactacagcaaggctgatgcctgggcagtgggagccatcgcctatgaaatcttcgggcttgtcaatcccttctacggccagggcaaggcccaccttgaaagccgcagctaccaagaggctcagctacctgcactgcccgagtcagtgcctccagacgtgagacagttggtgagggcactgctccagcgagaggccagcaagagaccatctgcccgagtagccgcaaatgtgcttcatctaagcctctggggtgaacatattctagccctgaagaatctgaagttagacaagatggttggctggctcctccaacaatcggccgccactttgttggccaacaggctcacagagaagtgttgtgtggaaacaaaaatgaagatgctctttctggctaacctggagtgtgaaacgctctgccaggcagccctcctcctctgctcatggagggcagccctgtgatgtccctgcatggagctggtgaattactaaaagaacatggcatcctctgtgtcgtgatggtctgtgaatggtgagggtgggagtcaggagacaagacagcgcagagagggctggttagccggaaaaggcctcgggcttggcaaatggaagaacttgagtgagagttcagtctgcagtcctctgctcacagacatctgaaaagtgaatggccaagctggtctagtagatgaggctggactgaggaggggtaggcctgcatccacagagaggatccaggccaaggcactggctgtcagtggcagagtttggctgtgacctttgcccctaacacgaggaactcgtttgaagggggcagcgtagcatgtctgatttgccacctggatgaaggcagacatcaacatgggtcagcacgttcagttacgggagtgggaaattacatgaggcctgggcctctgcgttcccaagctgtgcgttctggaccagctactgaattattaatctcacttagcgaaagtgacggatgagcagtaagtaagtaagtgtggggatttaaacttgagggtttccctcctgactagcctctcttacaggaattgtgaaatattaaatgcaaatttacaactgcagatgacgtatgtgccttgaactgaatatttggctttaagaatgattcttatactctgaaggtgagaatattttgtgggcaggtatcaacattggggaagagatttcatgtctaactaactaactttatacatgatttttaggaagctattgcctaaatcagcgtcaacatgcagtaaaggttgtcttcaactga

SEQ ID NO: 11 - DNA, 4178 bp, Homo sapiens
  misc_feature 1..4178: note = PRKN (cDNA - NCBI Reference Sequence: NM_004562.3)
  source 1..4178: mol_type = genomic DNA
aggatttaacccaggagagccgctggtgggaggcgcggctggcgccgctgcgcgcatgggcctgttcctggcccgcagccgccacctacccagtgaccatgatagtgtttgtcaggttcaactccagccatggtttcccagtggaggtcgattctgacaccagcatcttccagctcaaggaggtggttgctaagcgacagggggttccggctgaccagttgcgtgtgattttcgcagggaaggagctgaggaatgactggactgtgcagaattgtgacctggatcagcagagcattgttcacattgtgcagagaccgtggagaaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcttttatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgttgggaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtagccagggcggccgggcgccccatcgccacatcctgggggagcatacccagtgtctaccttcattttctaattctcttttcaaacacacacacacacgcgcgcgcgcgcacacacactcttcaagtttttttcaaagtccaactacagccaaattgcagaagaaactcctggatccctttcactatgtccatgaaaaacagcagagtaaaattacagaagaagctcctgaatccctttcagtttgtccacacaagacagcagagccatctgcgacaccaccaacaggcgttctcagcctccggatgacacaaataccagagcacagattcaagtgcaatccatgtatctgtatgggtcattctcacctgaattcgagacaggcagaatcagtagctggagagagagttctcacatttaatatcctgccttttaccttcagtaaacaccatgaagatgccattgacaaggtgtttctctgtaaaatgaactgcagtgggttctccaaactagattcatggctttaacagtaatgttcttatttaaattttcagaaagcatctattcccaaagaaccccaggcaatagtcaaaaacatttgtttatccttaagaattccatctatataaatcgcattaatgaaataccaactatgcgtaaatcaacttgtcacaaagtgagaaattatgaaagttaatttgaatgttgaatgtttgaattacagggaagaaatcaagttaatgtactttcattccctttcatgatttgcaactttagaaagaaattgtttttctgaaagtatcaccaaaaaatctatagtttgattctgagtattcattttgcaacttggagattttgctaatacatttggctccactgtaaatttaatagataaagtgcctataaaggaaacacgtttagaaatgatttcaaaatgatattcaatcttaacaaaagtgaacattattaaatcagaatctttaaagaggagcctttccagaactaccaaaatgaagacacgcccgactctctccatcagaagggtttatacccctttggcacaccctctctgtccaatctgcaagtcccagggagctctgcataccaggggttccccaggagagaccttctcttaggacagtaaactcactagaatattccttatgttgacatggattggatttcagttcaatcaaactttcagcttttttttcagccattcacaacacaatcaaaagattaacaacactgcatgcggcaaaccgcatgctcttacccacactacgcagaagagaaagtacaaccactatcttttgttctacctgtattgtctgacttctcaggaagatcgtgaacataactgagggcatgagtctcactagcacatggaggcccttttggatttagagactgtaaattattaaatcggcaacagggcttctctttttagatgtagcactgaaatccttgctggagggaagagaggggatgaactcaagttttccacatcctgggacacctgtccctcttttcctaactgcctaagataacccatttcttccaaccatctgaggacagtcccgtcgtctcagaggccctgcaccggggagagactgggctctgcagcagccacatcagcattcacagcttcatgtggcttcactgtctgaaaatctaccgactccaacatggccccacggtgacaacagacctgtgacaggaagcccaaagctcacatagaaatggtggacagatcaaagtctctatagtaagggaaaaaaagagaggtggcaggcatgagccccctgcacccagtggctcgtgtccatactgagtccagaccctgatcaaggcctgacttagtgtcactggcagtcccactaaattacacttccttacactggcccgatgcgacaaatcaggtggctcccttctgtcacgtggagcacacagtgttttccatcatccatagctttcttcctgatggtgtttgcattattgcgccttcccaatctgcatgctgcgttgggcttgcggtgcctgaacaaggtttgctcccatgagctcaggcaccctaggatcccctgttagactattaggctgtccagcatggtctcctttcccttcttggtggtggtcttttccctttccagaatagaacagtgattcttaaaataagttagagcaggccgggcgcggtggctcatgcctgtaatcccagcactttgggaggccgaggtgggtggatcacgaggtcaggagttcaagaccagcctggccaagatgatgaaaccccgtctctattaaaaatacaaaaattagctgggcgtggtggcaggcacctgtaatcccagcttcctgggaggctgaggcaggagaatcacttgaacccggggggcagaggttgcagtgagccgagatcacgccactgaactccagcctgggcaacagagtgagactctgtctcaaaaaaaaaaaaaaaacaaaaacaaaaaagcaagatcatccactacacatgaacatgaatcacagtattatttgcacaggaagggtgtaacaaaatatgaatgtatcaaaaaatagaaataaagtctttgcagaaaaagtctgtttttctctgaagtgtgttgagattatctgacaactctaagattgtacttaaattgtcaataaagcatcaaaagag